(19) United States
(12) Patent Application Publication
(10) Pub. No.: US 2004/0072871 A1
(43) Pub. Date: Apr. 15, 2004

(54) Novel thiophene derivatives, their process of preparation and the pharmaceutical compositions which comprise them

(76) Inventors: Anne-Claude Dublanchet, Fresnes Cedex (FR); Delphine Compere, Fresnes Cedex (FR); Philippe Cluzeau, Fresnes Cedex (FR); Stephane Blais, Fresnes Cedex (FR); Alexis Denis, Fresnes Cedex (FR); Pierre Ducrot, Fresnes Cedex (FR); Karine Courte, Fresnes Cedex (FR); Sophie Descamps, Fresnes Cedex (FR)

Correspondence Address: PFIZER INC., PATENT DEPARTMENT, MS8260-1611, EASTERN POINT ROAD, GROTON, CT, 06340, US

(21) Appl. No.: 10/638,016

(22) Filed: Aug. 8, 2003

(30) Foreign Application Priority Data
Aug. 13, 2002 (EP) 02292037.5

(51) Int. Cl. A61K 31/4436; C07D 333/38; A61K 31/381; C 07D 4 9/02

(52) U.S. Cl. 514/336; 514/438; 546/280.4; 549/71

(57) ABSTRACT

A compound of formula (I) selected from:

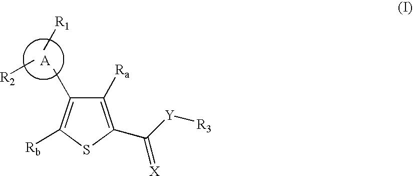

wherein:

X represents oxygen or sulphur,

Y represents oxygen, —NH— or —N(C₁-C₆)alkyl-,

Rₐ represents hydrogen, halogen, (C₁-C₃)alkyl, hydroxyl or (C₁-C₃)alkoxy,

R_{b} represents hydrogen, halogen or (C₁-C₃)alkyl,

A represents phenyl, pyridyl, (C₅-C₆)cycloalkyl or (C₅-C₆)cycloalkenyl,

R₁ and R₂ each represent a group selected from hydrogen, halogen, cyano, nitro, haloalkyl, haloalkoxy, alkyl, alkenyl, alkynyl, —OR₄, —NR₄R₅, —S(O)ₙR₄, —C(O)R₄, —CO₂R₄, —O—C(O)R₄, —C(O)NR₄R₅, —NR₅—C(O)R₄, —NR₅—SO₂R₄, -T-CN, -T-OR₄, -T-OCF₃, -T- NR₄R₅, -T-S(O)ₙR₄, -T-C(O)R₄, -T-CO₂R₄, -T-O—C(O)R₄, -T-C(O)NR₄R₅, -T-NR₄—C(O)R₅, -T-NR₄—SO₂R₅, —R₆ and -T-R₆ in which n, T, R₄, R₅ and R₆ are as defined in the description,

R₃ represents an —R₇ or —U—R₁₁ group in which R₇ represents hydrogen, alkyl, aryl, cycloalkyl or heterocycle, U represents a linear or branched alkylene chain and R₁₁ is defined in the description,

their optical isomers or their addition salts with a pharmaceutically acceptable acid or base, and their use as inhibitor of metalloproteinase and more specifically of metalloproteinase-12.

The present invention relates to novel thiophene derivatives, to their process of preparation and to the pharmaceutical compositions which comprise them.

The compounds of the present invention are particularly advantageous from a pharmacological viewpoint for their specific interaction with metalloproteinases and more specifically with macrophage metalloelastase (MMP-12) and are applied in the prevention and treatment of respiratory pathologies, such as chronic obstructive pulmonary disease (COPD), emphysema, chronic bronchitis, chronic pulmonary inflammation, asthma, cystic fibrosis, acute respiratory distress syndrome (ARDS), respiratory allergies, including allergic rhinitis, and diseases related to the production of TNFα, including severe fibrotic pulmonary diseases, pulmonary sarcoidosis and silicosis. The compounds of the present invention also show, at a lower level, an inhibitory activity for metalloproteinase-13 (MMP-13), rendering them potentially useful for the treatment of pathologies involving this enzyme, such as cancer, osteoporosis, osteoarthritis, arthritis, rheumatoid arthritis, atherosclerosis, multiple sclerosis or cardiac insufficiency.

Metalloproteinases (MMPs) constitute a large family of proteinases which decompose the extracellular matrix and are secreted in particular by mesenchymal cells, macrophages and polynuclear leukocytes. Metalloproteinases are classified into several subfamilies, depending on their primary structure and their specificity. These families include in particular collagenases (MMP-1, MMP-8 and MMP-13), stromelysins (MMP-3 and MMP-10), gelatinases (MMP-2 and MMP-9), matrilysin (MMP-7), macrophage metalloelastase 12 (MMP-12), and membrane-type MMPs (MMP-14, MMP-15, MMP-16 and MMP-17).

MMPs are zinc-metalloproteinases having the ability to decompose virtually all the components of the extracellular matrix, that is to say the interstitium and the basal membranes. An enhanced synthesis of these enzymes is found in numerous destructive diseases (inflammatory arthritis, atherosclerosis, tumour invasion, angiogenesis). MMPs (in particular those having a powerful elastolytic activity) are involved in the physiopathology of asthma and of chronic obstructive pulmonary disease, including smoking-related pulmonary emphysema (COPD).

Human macrophage elastase (HME or MMP-12) exhibits all the characteristics of the other MMPs. It decomposes numerous macromolecules of the extracellular matrix (gelatin, fibronectin and laminin) and in particular elastin. MMP-12 is not synthesized by circulating monocytic cells but only by macrophages or alternatively monocytes differentiated in vitro into macrophages. The pathology of emphysema is characterized by the destruction of elastin present in the walls of the pulmonary alveoli. The demonstration of the increase in the level of MMP-12 during the manifestation of this pathology thus suggests a predominant role of this enzyme in the onset and development of this disease. Likewise, studies have demonstrated the absence of development of emphysema in mice deficient in MMP-12, these mice being exposed for a lengthy period of time to cigarette smoke (Science, 1997, 277, 2002-2004). More recently, also using mice deficient in MMP-12 in a model of asthma, one group has suggested the involvement of MMP-12 in the development of chronic asthma (FASEB, 2002, 16, A590). These results clearly demonstrate that inhibitors of human macrophage elastase (MMP-12) might be very useful in the prevention and treatment of chronic respiratory pathologies, such as chronic obstructive pulmonary disease (COPD), emphysema, chronic bronchitis or chronic pulmonary inflammation, but also respiratory pathologies due to an inflammatory phenomenon, such as asthma, mucoviscidosis, acute respiratory distress syndrome (ARDS) or respiratory allergies, including allergic rhinitis, and diseases related to the production of TNFα, including severe fibrotic pulmonary diseases, pulmonary sarcoidosis and silicosis.

All the metalloproteinases exhibit a catalytic region composed of 162 to 173 amino acids comprising the reactive site of the enzyme. A Zn²⁺ ion is present in the active site, to which it is attached via histidine residues. This site constitutes one of the favoured attachment points for synthetic inhibitors of metalloproteinases as it makes it possible in particular to create a stable and strong chelation centre readily accessible for small molecules. Thus, all the powerful inhibitors decribed in the literature have a chemical functional group (such as a hydroxamic acid) which makes possible chelation between the zinc atom of the catalytic site of the metalloproteinase and the said inhibitor. This chelation ensures blocking of the active site and results in inhibition of the said enzyme.

One of the major problems of this type of inhibition is the absence of selectivity or the low degree of selectivity, this being because all the MMPs have a zinc ion within their active site. The second problem related to these powerful but generally not very selective inhibitors is the toxicity related to the presence of a chemical functional group, such as a hydroxamic acid.

One of the objects of the invention is thus to provide novel compounds having inhibitory properties with regard to type 12 metalloproteinase (MMP-12). A solution has been found by the preparation of novel thiophene derivatives, and by the use of the said compounds in pharmaceutical compositions capable of being used in the prevention and treatment of pathologies related to MMP-12.

Several patent applications or scientific articles describe compounds comprising a central thiophene unit. Mention may be made, among this literature, of Patent Application WO 98/23605, which discloses thien-2-ylcarboxamide derivatives substituted in the 4-position by a cyclic system and in the 5-position by a trifluoromethyl group. These compounds are claimed for their bactericidal and fungicidal activities. Patent Application WO 96/16954 also discloses compounds optionally comprising a 4-arylthien-2-ylcarboxamide system in which the amide functional group can be substituted by a phenyl group, which compounds are useful for their fungicidal property.

Patent Application WO 01/06821 claims compounds useful for the treatment of psychotic pathologies. These compounds, which constitute agonists of the nicotinic acetylcholine receptors, can in particular exhibit a central thien-2-ylcarboxamide unit in which the amide functional group is substituted by a 1-azabicyclo[2.2.2]oct-3-yl group. Mention may also be made of Patent Application JP 63175853 or of the paper Chem. Commun., 2001, 8, 759-760, which describe compounds comprising a substituted thiophene group, these compounds constituting fluorescence photoregulators or photographic developers.

None of these documents describes or suggests, for these compounds, an inhibitory activity with regard to MMP-12 and the potential use of this type of product in the treatment of respiratory pathologies, a novel property of the compounds claimed by the Applicant.

More particularly, the present invention relates to the compounds of formula (I):

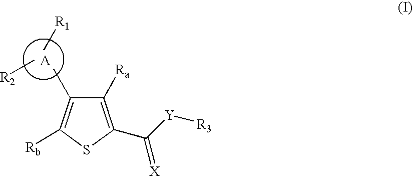

in which:

X represents an oxygen atom or a sulphur atom,

Y represents an oxygen atom, an —NH— group or an —N(C₁-C₆)alkyl group,

Rₐ represents a group selected from hydrogen, halogen, (C₁-C₃)alkyl, hydroxyl and (C₁-C₃)alkoxy,

R_{b} represents a group selected from hydrogen, halogen and (C₁-C₃)alkyl,

A represents a group selected from phenyl, pyridyl, (C₅-C₆)cycloalkyl and (C₅-C₆)cycloalkenyl,

R₁ and R₂, which are identical or different independently of each another, represent a group selected from:

hydrogen, halogen, cyano, nitro, halo(C₁-C₆)alkyl, halo(C₁-C₆)alkoxy, (C₁-C₆)alkyl, (C₂-C₆)alkenyl, (C₂-C₆)alkynyl,

—OR₄, —NR₄R₅, —S(O)ₙR₄, —C(O)R₄, —CO₂R₄, —O—C(O)R₄, —C(O)NR₄R₅, —NR₅—C(O)R₄, —NR₅—SO₂R₄, -T-CN, -T-OR₄, -T-OCF₃, -T-NR₄R₅, -T-S(O)ₙR₄, -T-C(O)R₄, -T-CO₂R₄, -T-O—C(O)R₄, -T-C(O)NR₄R₅, -T-NR₅—C(O)R₄, -T-NR₅—SO₂R₄, —R₆ and -T-R₆, in which:

n represents an integer from 0 to 2 inclusive,

T represents a linear or branched (C₁-C₆)alkylene chain optionally substituted by one group selected from oxo, halogen, (C₁-C₆)alkoxy, hydroxyl, amino, mono(C₁-C₆)alkylamino, di(C₁-C₆)alkylamino and/or in which optionally one of the carbon atoms is replaced by an oxygen atom, a sulphur atom, an —NH— group or an —N(C₁-C₆)alkyl- group (it being understood that, in the case where one of the carbon atoms is replaced by a group as defined above, then the said alkylene chain comprises at least one sequence of two atoms)

R₄ represents a hydrogen atom, a (C₁-C₆)alkyl group, a aryl group, a cycloalkyl group or a heterocycle,

R₅ represents a hydrogen atom or a (C₁-C₆)alkyl group,

R₆ represents a group selected from aryl, cycloalkyl and a heterocycle, each of these groups being optionally substituted from one to five identical or different groups selected, independently of each other, from halogen, cyano, nitro, oxo, halo(C₁-C₆)alkyl, halo(C₁-C₆)alkoxy, (C₁-C₆)alkyl, (C₁-C₆)alkenyl, —OR₄₀, —NR₄₀R₅₀, —S(O)ₙ₁R₄₀, —C(O)R₄₀, —CO₂R₄₀, —O—C(O)R₄₀, —C(O)NR₄₀R₅₀, —NR₅₀—C(O)R₄₀, —NR₅₀—SO₂R₄₀, -T₁-CN, -T₁-OR₄₀, -T₁-OCF₃, -T₁-NR₄₀R₅₀, -T₁-S(O)ₙR₄₀, -T₁-C(O)R₄₀, -T₁-CO₂R₄₀, -T₁-O—C(O)R₄₀, -T₁-C(O)NR₄₀R₅₀, -T₁-NR₅₀—C(O)R₄₀, -T₁-NR₅₀—SO₂R₄₀, -G₁ and -T₁-G₁, in which:

R₄₀, R₅₀, T₁ and n₁ respectively have the same meanings as R₄, R₅, T and n as defined above,

G₁ represents a group selected from aryl, cycloalkyl and a heterocycle, each of these groups optionally being substituted by 1 to 5 identical or different groups selected, independently of each other, from halogen, cyano, nitro, halo(C₁-C₆)alkyl, halo(C₁-C₆)alkoxy, (C₁-C₆)alkyl, hydroxyl, (C₁-C₆)alkoxy, phenoxy, benzyloxy, amino, mono(C₁-C₆)alkylamino, di(C₁-C₆)alkylamino, mercapto, (C₁-C₆)alkylthio, (C₁-C₇)acyl, (C₁-C₆)alkylsulphinyl, carboxyl, (C₁-C₆)alkoxycarbonyl, phenyl and a heterocycle,

R₃ represents an —R₇ or —U—R₁₁ group, in which:

R₇ represents a group selected from hydrogen, (C₁-C₆)alkyl, aryl, cycloalkyl and heterocycle, each cyclic system optionally being substituted by one to five identical or different groups selected, independently of each other, from halogen, cyano, nitro, halo(C₁-C₆)alkyl, halo(C₁-C₆)alkoxy, (C₁-C₆)alkyl, —OR₈, —NR₈R₉, —S(O)ₘR₈, —C(O)R₈, —CO₂R₈, —O—C(O)R₈, —C(O)NR₈R₉, —NR₉—C(O)R₈, —NR₉—SO₂R₈, —V—CN, —V—OR₈, —V—NR₈R₉, —V—S(O)ₘR₈, —V—C(O)R₈, —V—CO₂R₈, —V—O—C(O)R₈, —V—C(O)NR₈R₉, —V—NR₉—C(O)R₈, —V—NR₉—SO₂R₈, —R₁₀ and —V—R₁₀, in which:

m represents an integer from 0 to 2 inclusive,

V represents a group selected from a linear or branched (C₁-C₆)alkylene chain, a linear or branched (C₂-C₆)alkenylene chain, a cyclopropylene group and a linear or branched (C₂-C₆)alkylene chain in which one of the carbon atoms is replaced by an oxygen agom, a sulphur atom, an —NH— group or an —N(C₁-C₆)alkyl- group,

R₈ represents a hydrogen atom, a (C₁-C₆)alkyl, aryl, cycloalkyl group or a heterocycle,

R₉ represents a hydrogen atom or a (C₁-C₆)alkyl group,

R₁₀ represents an aryl group, a cycloalkyl group or a heterocycle,

U represents a linear or branched (C₁-C₆)alkylene chain optionally substituted by one hydroxyl group or a linear or branched (C₂-C₆)alkylene chain in which one of the carbon atoms is replaced by an oxygen atom, a sulphur atom, an —NH— group or an —N(C₁-C₆)alkyl- group,

R₁₁ represents a group selected from halogen, —OR₁₂, —NR₁₂R₁₃, —S(O)ₚR₁₂, —C(O)R₁₂, —CO₂R₁₂, —O—C(O)R₁₂, —C(O)NR₁₂R₁₃, —NR₁₃—C(O)R₁₂, —NR₁₃—SO₂R₁₂ and —R₁₄, the latter group optionally being substituted by one to three identical or different groups selected, independently of each other, from halogen, cyano, nitro, halo(C₁-C₆)alkyl, halo(C₁-C₆)alkoxy, (C₁-C₆)alkyl, (C₂-C₆)alkenyl, (C₂-C₆)alkynyl, —OR₁₅, —NR₁₅R₁₆, —S(O)_{q}R₁₅, —C(O)R₁₅, —O—C(O)R₁₅, —CO₂R₁₅, —C(O)NR₁₅R₁₆, —NR₁₆—C(O)R₁₅, —NR₁₆—SO₂R₁₅, —R₁₇, —W—CN, —W—OR₁₅, —W—NR₁₅R₁₆, —W—S(O)_{q}R₁₅, —W—C(O)R₁₅, —W—CO₂R₁₅, —W—O—C(O)R₁₅, —W—C(O)NR₁₅R₁₆, —W—NR₁₆—C(O)R₁₅, —W—NR₁₆—SO₂R₁₅, —W—R₁₇ and —C(O)—W₁—CO₂R₁₅, in which:

R₁₂ represents a hydrogen atom, a (C₁-C₆)alkyl group, an aryl group, a cycloalkyl group or a heterocycle,

R₁₃ represents a hydrogen atom or a (C₁-C₆)alkyl group,

R₁₄ represents an aryl group, a cycloalkyl group or a heterocycle,

R₁₅ represents a hydrogen atom, a (C₁-C₆)alkyl, a aryl group, a cycloalkyl group or a heterocycle,

R₁₆ represents a hydrogen atom or a (C₁-C₆)alkyl group,

R₁₇ represents an aryl group, a cycloalkyl group or a heterocycle,

p represents an integer from 0 to 2 inclusive,

q represents an integer from 0 to 2 inclusive,

W represents a group selected from a linear or branched (C₁-C₆)alkylene chain, a linear or branched (C₂-C₆)alkenylene chain, a cyclopropylene group and a linear or branched (C₂-C₆)alkylene chain in which one of the carbon atoms is replaced by an oxygen atom, a sulphur atom, an —NH— group or an —N(C₁-C₆)alkyl- group,

W₁ represents a linear or branched (C₁-C₆)alkylene chain,

to their isomers, and to their addition salts with a pharmaceutically acceptable acid or base, it being understood that the compounds of formula (I) are not selected from:

ethyl 5-methyl-4-phenylthiophen-2-ylcarboxylate,

5-methyl-4-phenylthiophen-2-ylcarboxylic acid,

methyl 3-hydroxy-4-phenylthiophen-2-ylcarboxylate,

methyl 3-methoxy-4-phenylthiophen-2-ylcarboxylate,

3-methoxy-4-phenylthiophen-2-ylcarboxylic acid,

methyl 4-(4-methoxyphenyl)thiophen-2-ylcarboxylate,

methyl 4-phenylthiophen-2-ylcarboxylate,

4-(4-methoxyphenyl)thiophen-2-ylcarboxylic acid,

4-phenylthiophen-2-ylcarboxylic acid,

4-(4-tert-butylphenyl)thiophen-2-ylcarboxylic acid,

methyl 5-chloro-3-hydroxy-4-phenylthiophen-2-ylcarboxylate,

4-(3,5-dimethylphenyl)thiophen-2-ylcarboxylic acid,

methyl 4-[4-(acetylamino)phenyl]thiophen-2-ylcarboxylate,

ethyl 4-phenylthiophen-2-ylcarboxylate,

4-phenylthiophen-2-ylcarboxamide,

N-methyl-4-phenylthiophen-2-ylcarboxamide,

N,N-dimethyl-4-phenylthiophen-2-ylcarboxamide,

5-methyl-4-phenylthiophen-2-ylcarboxamide,

N-methyl-5-methyl-4-phenylthiophen-2-ylcarboxamide,

N,N-dimethyl-5-methyl-4-phenylthiophen-2-ylcarboxamide,

methyl 2-{[4-(4-methoxyphenyl)thiophen-2-yl]carboxamido}benzoate,

N-[3-(trifluoromethyl)phenyl]-4-(4-methoxyphenyl)thiophen-2-ylcarboxamide,

methyl 4-(2-bromophenyl)thiophen-2-ylcarboxylate,

methyl 4-(3-bromophenyl)thiophen-2-ylcarboxylate,

methyl 4-(4-bromophenyl)thiophen-2-ylcarboxylate,

N-(2-methoxyethyl)-4-(4-methoxyphenyl)thiophen-2-ylcarboxamide,

N-isopropyl-4-(4-methoxyphenyl)thiophen-2-ylcarboxamide,

4-(4-methoxyphenyl)-N-(2-morpholinoethyl)thiophen-2-ylcarboxamide,

4-(4-chlorophenyl)-N-(2-methoxyethyl)thiophen-2-ylcarboxamide,

4-(4-chlorophenyl)-N-(2-morpholinoethyl)thiophen-2-ylcarboxamide,

4-(4-chlorophenyl)-N-(2-phenylethyl)thiophen-2-ylcarboxamide,

4-(4-chlorophenyl)-N-(tetrahydrofurylmethyl)thiophen-2-ylcarboxamide,

and 4-(4-methoxyphenyl)-N-(tetrahydrofurylmethyl)thiophen-2-ylcarboxamide;

it also being understood that:

if Rₐ represents a hydrogen atom,A represents a cyclopenten-1-yl group substituted in the 2 position by an R₁ group taking the definition thienyl optionally substituted, then R_{b} represents a group selected from hydrogen, halogen and (C₂-C₃)alkyl,

if R₃ represents an R₇ group taking the definition heterocycle, then the said heterocycle cannot represent a l-azabicyclo[2.2.2]oct-3-yl group,

and if R₃ represents an R₇ group taking the definition phenyl substituted in the para position by an R₁₀ group, then the said R₁₀ group cannot represent a 5-methyl-4,5-dihydro-3-oxo-2H-pyridazin-6-yl group,

it also being understood that, in the general definition of the various groups of the compounds of formula (I):

an aryl group denotes an aromatic monocyclic or bicyclic system comprising from 4 to 10 carbon atoms, it being understood that, in the case of a bicyclic system, one of the rings exhibits an aromatic character and the other ring is aromatic or unsaturated; mention may be made, by way of indication, of the following groups: phenyl, naphthyl, indenyl, benzocyclobutenyl, 1,2,3,4-tetrahydronaphthyl, and the like,

a cycloalkyl group denotes a saturated or partially unsaturated, fused or bridged, bicyclic or monocyclic system comprising from 3 to 12 carbon atoms; mention may be made, by way of indication, of the following groups: cyclopropyl, cyclobutyl, cyclopentyl, cyclohexyl, cycloheptyl, cyclooctyl, adamantyl, decalinyl, norbornyl, cyclopentenyl, cyclohexenyl, cyclohexenediyl, and the like,

a heterocycle denotes a saturated, unsaturated or aromatic, 3- to 12-membered, fused or bridged, bicyclic or monocyclic system comprising from 1 to 4 identical or different heteroatoms selected, independently of each other, from oxygen, sulphur and nitrogen and optionally comprising 1 or 2 oxo or thioxo groups, it being understood that, in the case of a bicyclic system, one of the rings can exhibit an aromatic nature and the other ring is aromatic or unsaturated, or both rings are saturated, or one of the rings is saturated and the other ring is unsaturated, or both rings are unsaturated; mention may be made, by way of indication, of the following groups: furyl, thienyl, pyrrolyl, pyrazolyl, pyridyl, pyrimidyl, pyrazinyl, benzofuryl, benzothienyl, indolyl, quinolyl, isoquinolyl, imidazolyl, benzodioxolyl, benzodioxinyl, benzo[1,2,5]thiadiazolyl, benzo[1,2,5]oxadiazolyl, [1,2,3]triazolyl, [1,2,4]triazolyl, morpholinyl, piperidyl, piperazinyl, pyrrolidinyl, and the like,

a “(C₁-C₆)alkyl group” denotes a linear or branched carbonaceous chain comprising from 1 to 6 carbon atoms; mention may be made, by way of indication, of the following groups: methyl, ethyl, propyl, isopropyl, tert-butyl, neopentyl, hexyl, and the like,

a “(C₂-C₆)alkenyl group” denotes a linear or branched carbonaceous chain comprising from 2 to 6 carbon atoms and one or more double bonds; mention may be made, by way of indication, of the following groups: vinyl, allyl, 3-buten-1-yl, 2-methyl-buten-1-yl, hexenyl, and the like,

a “(C₂-C₆)alkynyl group” denotes a linear or branched carbonaceous chain comprising from 2 to 6 carbon atoms and one or more triple bonds; mention may be made, by way of indication, of the following groups: ethynyl, propynyl, 3-butyn-1-yl, 2-methylbutyn-1-yl, hexynyl, and the like,

a “(C₁-C₆)alkoxy group” denotes an alkyl group as defined above bonded to an oxygen atom; mention may be made, by way of indication, of the following groups: methoxy, ethoxy, n-propyloxy, tert-butyloxy, and the like,

a “halo(C₁-C₆)alkyl group” denotes a linear or branched carbonaceous chain comprising from 1 to 6 carbon atoms and substituted by 1 to 6 halogen atoms; mention may be made, by way of indication, of the following groups: trifluoromethyl, 2,2,2-trifluoroethyl, and the like,

a “halo(C₁-C₆)alkoxy group” denotes a linear or branched carbonaceous chain comprising from 1 to 6 carbon atoms and substituted by 1 to 6 halogen atoms, the said chain being connected to the compound of formula (I) by an oxygen atom; mention may be made, by way of indication, of the following groups: trifluoromethoxy, 2,2,2-trifluoroethoxy, and the like,

a “halogen atom” denotes an atom selected from bromine, chlorine, fluorine and iodine,

a “acyl group” denotes a hydrogen atom, an alkyl group as defined above, a cycloalkyl comprising 3 to 6 carbon atoms or a phenyl group bonded through an oxo group to the compounds of formula (I); mention may be made, by way of indication, of the following groups: formyl, acetyl, ethylcarbonyl, n-propylcarbonyl, tert-butylcarbonyl, cyclopropylcarbonyl, benzoyl, and the like,

a “cyclic system” denotes the aryl and cycloalkyl groups and the heterocycles as defined above,

optical isomers refer to racemates, enantiomers and diastereoisomers.

More specifically, the present invention relates to compounds of formula (I) as defined above in which A represents a group selected from phenyl, (C₅-C₆)cycloalkyl and (C₅-C₆)cycloalkenyl, R₁, R₂, R₃, Rₐ, R_{b}, X and Y being as defined above in the formula (I).

According to an advantageous alternative form of the invention, the preferred compounds of the invention are the compounds of formula (IA):

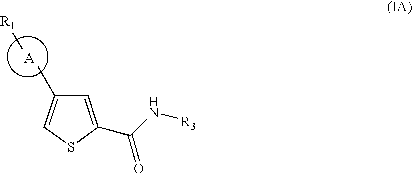

in which A, R₁ and R₃ are as defined in the formula (I).

Advantageously, A preferably represents a phenyl group in the compounds of formula (I) or of formula (IA).

In a particularly advantageous way, the preferred compounds of the invention are the compounds of formula (I) as defined above in which A represents a phenyl group, Rₐ represents a hydrogen atom, R_{b} represents a hydrogen atom, X represents an oxygen atom, Y represents an —NH— group, R₁ is as defined in the general formula (I), R₂ represents a hydrogen atom and R₃ represents a —U—R₁₁ group in which U and R₁₁ are as defined in the general definition of the formula (I).

According to a particularly advantageous alternative form of the invention, the said A group taking the definition phenyl is substituted by an R₁ group as defined in the formula (I) situated in the para position.

The preferred R₁ groups according to the invention are the groups selected from trifluoromethyl, trifluoromethoxy, 2,2,2-trifluoroethoxy, (C₁-C₆)alkyl, cyano, nitro, —OR₄, —SR₄, —NR₄R₅, —CO₂R₄, —C(O)R₄, -T-CO₂R₄, -T-OH, -T-CN, -T-R₆ and —R₆ in which:

R₄ represents a hydrogen atom, a (C₁-C₆)alkyl group, an aryl group, a cycloalkyl group or a heterocycle,

R₅ represents a hydrogen atom or a (C₁-C₆)alkyl group,

R₆ represents a group selected from aryl, cycloalkyl and a heterocycle, each of these groups optionally being substituted by one or two identical or different groups selected from halogen, cyano, nitro, trifluoromethyl, halo(C₁-C₆)alkoxy, (C₁-C₆)alkyl, vinyl, —OR₄₀, —NR₄₀R₅₀, —S(O)ₙ₁R₄₀, —C(O)R₄₀, —CO₂R₄₀, —O—C(O)R₄₀, —C(O)NR₄₀R₅₀, —NR₅₀—C(O)R₄₀, —NR₅₀—SO₂R₄₀, -T₁-C(O)R₄₀, -T₁-CN, -T₁-OR₄₀ and -T₁-CO₂R₄₀, in which R₄₀, R₅₀, T₁ and n₁ are as defined in the formula (I),

T represents a —CH₂— or —CH₂—O— group in which the oxygen atom is connected to the A group of the compounds of formula (I).

Preferably, R₁ represents a group selected from (C₂-C₄)alkyl, (C₁-C₄)alkylthio, (C₁-C₄)alkoxy, trifluoromethoxy and —R₆ in which R₆ represents a group selected from phenyl optionally substituted by one or two groups as defined in the formula (I), cyclohexyl and a 5- or 6-membered heterocycle comprising from 1 to 3 heteroatoms selected from oxygen, nitrogen and sulphur.

According to another particularly advantageous alternative form of the invention, R₁ represents a group selected from:

phenyl optionally substituted by a group selected from halogen, hydroxyl, (C₁-C₄)alkoxy, phenoxy, trifluoromethoxy, acyl, (C₁-C₄)alkylsulphonyl, -T-CO₂R₄₀ and -T-CN in which T and R₄₀ are as defined in the formula (I),

cyclohexyl,

4-pyridyl, 3-pyridyl, 5-pyrimidyl, N-pyrrolidinyl, 1-methylpyrrol-3-yl, 3,6-dihydro-2H-pyridin-1-yl and 2-hydroxy-4-pyridyl.

Advantageously, R₃ represents an R₇ group selected from phenyl, cyclohexyl and pyridyl, each of these groups optionally being substituted by one or two identical or different groups selected, independently of each other, from (C₁-C₆)alkyl, —OR₈, —NR₈R₉, —CO₂R₈, —V—OR₈, —V—NR₈R₉ and —V—CO₂R₈ in which V represents a linear or branched (C₁-C₄)alkylene chain or a linear or branched (C₂-C₄)alkenylene chain, R₈ represents a hydrogen atom or a (C₁-C₆)alkyl group, and R₉ represents a hydrogen atom.

According to a preferred alternative form of the invention, R₃ represents a —U—R₁₁ group in which U represents a linear or branched (C₁-C₄)alkylene chain and R₁₁ represents a group selected from —CO₂R₁₂ and —R₁₄ in which:

R₁₂ represents a hydrogen atom or a (C₁-C₆)alkyl group,

R₁₄ represents a group selected from phenyl, cyclohexyl, morpholinyl and pyridyl, each of these groups optionally being substituted by one or two identical or different groups selected, independently of each other, from halogen, (C₁-C₆)alkyl, —CO₂R₁₅ and —W—CO₂R₁₅, in which R₁₅ represents a hydrogen atom or a (C₁-C₆)alkyl group and W represents a linear or branched (C₁-C₆)alkylene chain, or a linear or branched (C₂-C₆)alkenylene chain.

The isomers, and the addition salts with a pharmaceutically acceptable acid or base, of the preferred alternative forms and compounds form an integral part of the invention.

The invention also relates to the pharmaceutically acceptable salts of the compounds of formula (I). A review of the pharmaceutically acceptable salts is described in particular in J. Pharm. Sci., 1977, 66, 1-19.

Pharmaceutically acceptable acids mean nontoxic organic or mineral acids. Mention may be made, among pharmaceutically acceptable acids, without implied limitation, of hydrochloric, hydrobromic, sulphuric, phosphonic, nitric, acetic, trifluoroacetic, lactic, pyruvic, malonic, succinic, glutaric, fumaric, tartaric, maleic, citric, ascorbic, oxalic, methanesulphonic, camphoric, benzoic or toluenesulphonic acids, and the like.

Pharmaceutically acceptable bases mean nontoxic organic or mineral bases.

Mention may be made, among pharmaceutically accepable bases, without implied limitation, of sodium hydroxide, potassium hydroxide, lithium hydroxide, calcium hydroxide, triethylamine, tert-butylamine, 2-diethylaminoethanol, ethanolamine, ethylenediamine, dibenzylethylenediamine, piperidine, pyrrolidine, morpholine, piperazine, benzylamine, arginine, lysine, histidine, glucamine, glucosamine, quaternary ammonium hydroxides, and the like.

Generally, the term “isomers” of the compounds of the invention denotes the optical isomers, such as the enantiomers and the diastereoisomers. More particularly, the pure enantiomeric forms of the compounds of the invention can be separated from the mixtures of enantiomers, which are reacted with a releasable agent for resolving the racemates, the said agent for its part existing in the form of a pure enantiomer, making it possible to obtain the corresponding diastereoisomers. These diastereoisomers are subsequently separated according to separating techniques well known to a person skilled in the art, such as crystallization or chromatography, and then the resolving agent is removed using conventional techniques of organic chemistry, to yield to a pure enantiomer being obtained. In another way, the pure enantiomeric forms of the compounds of the invention can be separated by chromatography on a chiral column.

The compounds of the invention, which are present in the form of a mixture of diastereoisomers, are isolated in the pure form by the use of conventional separating techniques, such as chromatographic techniques.

In some specific cases, the process for the separation of the compounds of the invention can result in the predominant formation of one enantiomer or of one diastereoisomer with respect to the other.

The invention also relates to a process for the preparation of compounds of formula (I). More particularly, the compounds of formula (I) can be obtained from the compounds of formula (II):

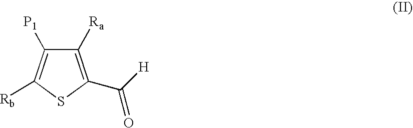

in which Rₐ and R_{b} are as defined in the formula (I) and P₁ represents a halogen atom or a triflate group,

which compounds of formula (II) are subjected to oxidation conditions in the presence, for example, of silver nitrate in a basic and polar medium, to give the compounds of formula (III):

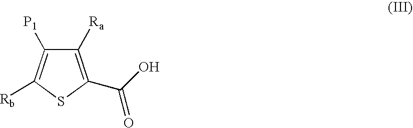

in which Rₐ, R_{b} and P₁ are as defined above,

which compounds of formula (III) are optionally converted to the corresponding acid chlorides (IV) by reaction with oxalyl chloride, for example,

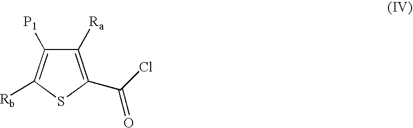

in which Rₐ, R_{b} and P₁ are as defined above,

or which compounds of formula (III) are treated directly, under peptide coupling conditions in the presence, for example, of HATU (O-(7-azabenzotriazol-1-yl)-N,N,N′,N′-tetramethyluronium hexafluorophosphate) and in a basic medium, with a compound of formula (V):

HY—R₃ (V)

in which Y and R₃ have the same meanings as in the compounds of formula (I), to yield to the compounds of formula (VI):

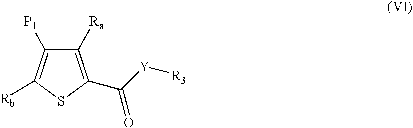

in which Rₐ, R_{b}, R₃, Y and P₁ are as defined above,

which compounds of formula (VI) are:

either reacted, under basic palladium coupling conditions, with a compound of formula (VII):

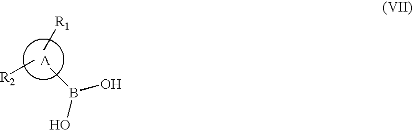

in which A, R₁ and R₂ have the same meanings as in the compounds of formula (I), to yield to the compounds of formula (I/a), a particular case of the compounds of formula (I):

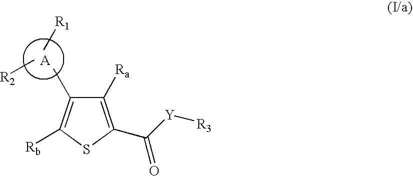

in which Rₐ, R_{b}, R₁, R₂, R₃, Y and A are as defined above,

or treated with hexamethylditin, in the presence of a palladium catalyst, to yield to the compounds of formula (VIII):

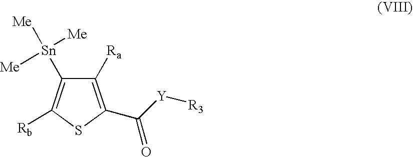

in which Rₐ, R_{b}, R₃ and Y are as defined above,

which componds of formula (VIII) are reacted:

with a compound of formula (IX):

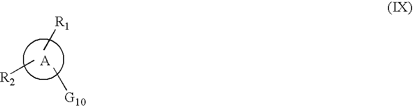

in which R₁, R₂ and A have the same meanings as in the formula (I) and G₁₀ represents a halogen atom selected from chlorine and bromine or a triflate group,

either in the presence of triphenylphosphinearsenic and of a palladium catalyst, in the case where G₁₀ represents a triflate group,

or in the presence of a cupric halide compound, such as CuBr₂, and of a palladium catalyst, under polar solvent conditions, in the case where G₁₀ represents a halogen atom,

to also yield to the compounds of formula (I/a):

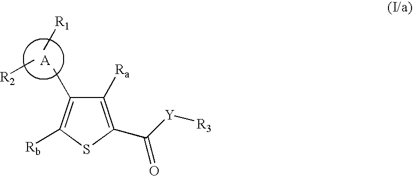

in which Rₐ, R_{b}, R₁, R₂, R₃, Y and A are as defined above,

or treated with bis(pinacolato)diborane, followed by an oxidation reaction by reaction, for example, with sodium periodate, to yield to the compounds of formula (VIa):

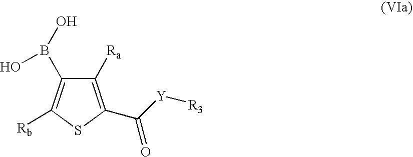

in which Rₐ, R_{b}, R₃ and Y are as defined above,

which compounds of formula (VIa) are reacted, under basic palladium coupling conditions, with a compound of the formula (IX):

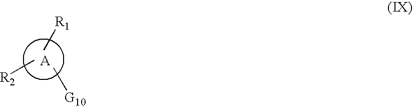

in which A, R₁, R₂ and G₁₀ are as defined above,

to also yield to the compounds of formula (I/a) as defined above:

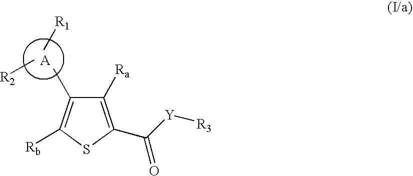

in which Rₐ, R_{b}, R₁, R₂, R₃, Y and A are as defined above,

or reacted with a compound of formula (IXa) (the said compounds of formula (IXa) being obtained by treatment of the compounds of formula (IX) as defined above with hexamethylditin in the presence of a palladium catalyst):

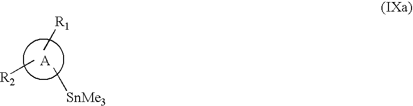

in which A, R₁ and R₂ are as defined in the formula (I),

either in the presence of triphenylphosphinearsenic and of a palladium catalyst, in the case where P₁ in the compounds of formula (VI) represents a triflate group,

or in the presence of a cupric halide compound, such as CuBr₂, and of a palladium catalyst, under polar solvent conditions, in the case where P₁ in the compounds of formula (VI) represents a halogen atom,

to also yield to the compounds of formula (I/a) as described above,

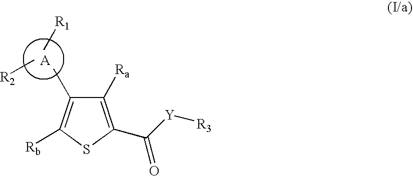

in which Rₐ, R_{b}, R₁, R₂, R₃, Y and A are as defined above,

which compounds of formula (I/a), in the specific case where they represent compounds of formula (I/b), in which A represents a phenyl group, R₂ represents a hydrogen atom, R₁ represents a hydroxyl group or a halogen atom (Hal) and Rₐ, R_{b}, Y and R₃ have the same meanings as in the formula (I):

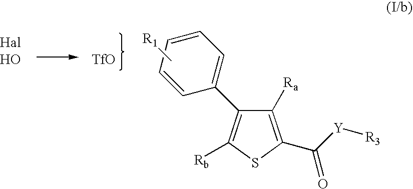

compounds of formula (I/b) which can be treated beforehand with trifluoromethanesulphonic anhydride in the presence of a strong base, in the case where R₁ represents a hydroxyl group, to produce the triflate activated derivative,

the said compounds, carrying an R₁ group taking the definition halogen or triflate, could then:

either to be reacted under basic conditions and in the presence of a palladium catalyst with a compound of formula (X):

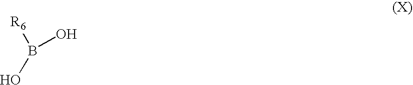

in which R₆ is as defined in the formula (I), that is to say that they represent a group selected from aryl, cycloalkyl and a heterocycle, each optionally being substituted,

to yield to the compounds of formula (I/c), a particular case of the compounds of formula (I):

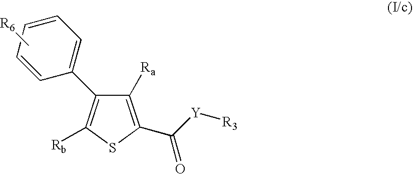

in which R₆, Rₐ, R_{b}, Y and R₃ are as defined above,

or to be treated with bis(pinacolato)diborane, followed by an oxidation reaction, for example, with sodium periodate, to yield to the compounds of formula (XI):

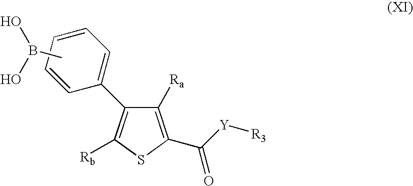

in which Rₐ, R_{b}, Y and R₃ are as defined above,

which compounds of formula (XI) are reacted under basic conditions and in the presence of a palladium catalyst with a compound of formula (Xa):

R₆—P₂ (Xa)

in which R₆ is as defined in the formula (I), that is to say that it represents a group selected from aryl, cycloalkyl and a heterocycle, each optionally being substituted, and P₂ represents a halogen atom or a triflate group,

to also yield to the compounds of formula (I/c) as defined above:

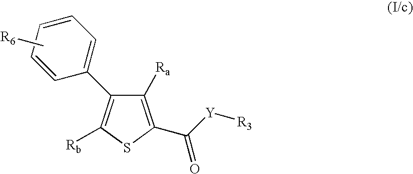

in which R₆, Rₐ, R_{b}, Y and R₃ are as defined above,

or to be treated with hexamethylditin in the presence of a palladium catalyst, to yield to the compounds of formula (XIa):

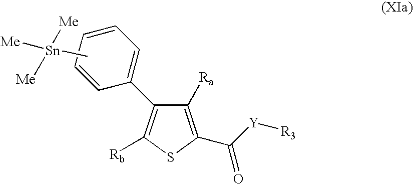

in which Rₐ, R_{b}, R₃ and Y are as defined above,

which compounds of formula (XIa) are reacted with a compound of formula (Xa) as defined above:

R₆—P₂ (Xa)

in which R₆ is as defined in the formula (I) and P₂ represents a halogen atom or a triflate group,

either in the presence of triphenylphosphinearsenic and of a palladium catalyst, in the case where P₂ represents a triflate group,

or in the presence of a cupric halide compound, such as CuBr₂, and of a palladium catalyst, under polar solvent conditions, in the case where P₂ represents a halogen atom,

to also yield to the compounds of formula (I/c) as defined above:

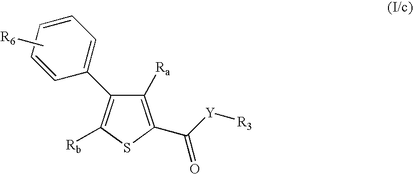

in which R₆, Rₐ, R_{b}, Y and R₃ are as defined above,

or to be reacted with a compound of formula (Xb) (the said compounds of formula (Xb) being obtained by treatment of the compounds of formula (Xa) as defined above with hexamethylditin in the presence of a palladium catalyst):

R₆—SnMe₃ (Xb)

in which R₆ is as defined above,

either in the presence of triphenylphosphinearsenic and of a palladium catalyst, in the case where the compounds of formula (I/b) comprise a triflate group,

or in the presence of a cupric halide compound, such as CuBr₂, and of a palladium catalyst, under polar solvent conditions, in the case where the compounds of formula (I/b) comprise a halogen atom,

to also yield to the compounds of formula (I/c) as described above,

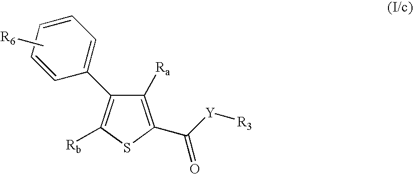

in which R₆, Rₐ, R_{b}, Y and R₃ are as defined above,

or to be reacted under palladium coupling conditions, in a basic medium, with a compound of formula (XII):

R_{6′}—H (XII)

in which R_{6′} represents a nitrogenous heterocycle optionally substituted by one or more groups such as are defined for the substituents of the R₆ group within the compounds of formula (I),

to yield to the compounds of formula (I/d), a particular case of the compounds of formula (I):

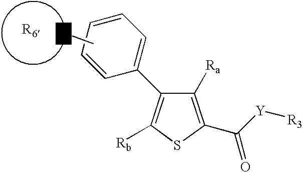

in which Rₐ, R_{b}, Y and R₃ are as defined above and R_{6′} represents an optionally substituted nitrogenous heterocycle as defined in the formula (I),

the compounds of formulae (I/a), (I/b), (I/c) and (I/d) together forming the compounds of formula (I/e):

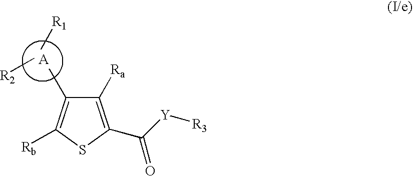

in which Rₐ, R_{b}, R₁, R₂, A, Y and R₃ are as defined in the formula (I),

which compounds of formula (I/e) can be treated, for example with 2,4-bis(4-methoxyphenyl)-1,3-dithia-2,4-diphosphetane-2,4-disulphide, to yield to the compounds of formula (I/f), a particular case of the compounds of formula (I):

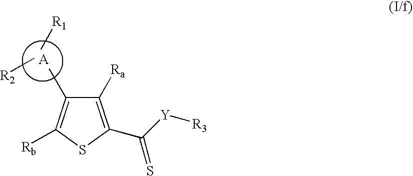

in which Rₐ, R_{b}, R₁, R₂, A, Y and R₃ are as defined in the formula (I),

the compounds (I/a) to (I/f) representing all the compounds of the invention, which are purified, if appropriate, according to a conventional purification technique, which can, if desired, be separated into their various isomers according to a conventional separating technique, and which are converted, if appropriate, into their addition salts with a pharmaceutically acceptable acid or base.

The compounds of formulae (II), (V), (VII), (IX), (X) and (Xa) are either commercial compounds or are obtained according to known methods of organic synthesis readily accessible and comprehensible to a person skilled in the art.

According to an alternative form of the invention, the compounds of formula (I) can also be obtained by a second preparation process, characterized in that use is made, as starting material, of a compound of formula (II):

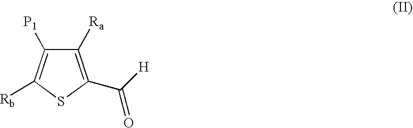

in which Rₐ and R_{b} are as defined in the formula (I) and P₁ represents a halogen atom or a triflate group,

which compounds of formula (II) are subjected to oxidation conditions in the presence, for example, of silver nitrate in a basic and polar medium, to give the compounds of formula (III):

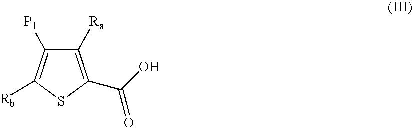

in which Rₐ, R_{b} and P₁ are as defined above,

the acid functional group of compounds of formula (III) is esterified by the action of an alcohol in the presence of a strong acid, to yield to the compounds of formula (XX):

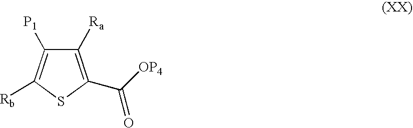

in which Rₐ, R_{b} and P₁ are as defined above and P₄ represents a linear or branched (C₁-C₄)alkyl group,

which compounds of formula (XX) are:

either reacted, under basic palladium coupling conditions, with a compound of formula (VII):

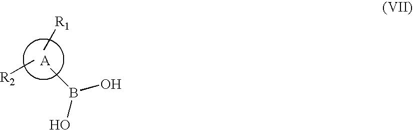

in which A, R₁ and R₂ have the same meanings as in the compounds of formula (I), to yield to the compounds of formula (I/g), a particular case of the compounds of formula (I):

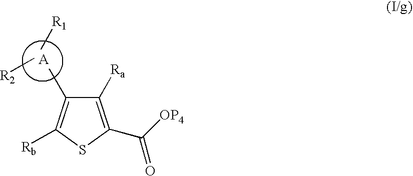

in which Rₐ, R_{b}, R₁, R₂, A and P₄ are as defined above,

or treated with hexamethylditin in the presence of a palladium catalyst, to yield to the compounds of formula (XXI):

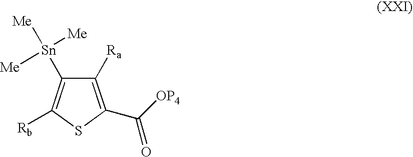

in which Rₐ, R_{b} and P₄ are as defined above,

which compounds of formula (XXI) are reacted:

with a compound of formula (IX):

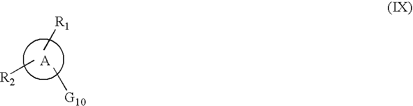

in which R₁, R₂ and A have the same meanings as in the formula (I) and G₁₀ represents a halogen atom selected from chlorine and bromine or a triflate group,

either in the presence of triphenylphosphinearsenic and of a palladium catalyst, in the case where G₁₀ represents a triflate group,

or in the presence of a cupric halide compound, such as CuBr₂, and of a palladium catalyst, under polar solvent conditions, in the case where G₁₀ represents a halogen atom,

to also yield to the compounds of formula (I/g):

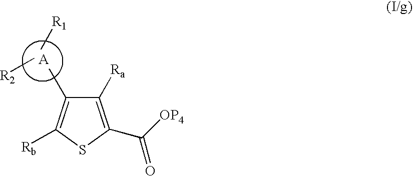

in which Rₐ, R_{b}, R₁, R₂, A and P₄ are as defined above,

or treated with bis(pinacolato)diborane, followed by an oxidation reaction, to yield to the compounds of formula (XXII):

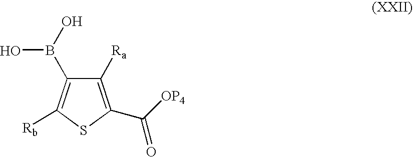

in which Rₐ, R_{b} and P₄ are as defined above,

which compounds of formula (XXII) are reacted under basic palladium coupling conditions with a compound of formula (IX):

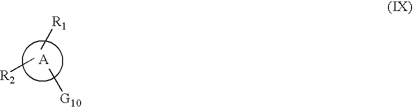

in which R₁, R₂, G₁₀ and A are as defined above,

to also yield to the compounds of formula (I/g) as defined above:

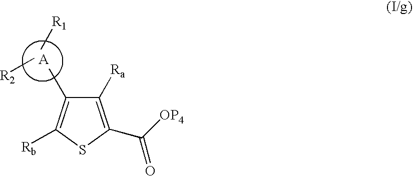

in which Rₐ, R_{b}, R₁, R₂ and P₄ are as defined above,

or reacted with a compound of formula (IXa) (the said compounds of formula (IXa) being obtained by treatment of the compounds of formula (IX) as defined above with hexamethylditin in the presence of a palladium catalyst):

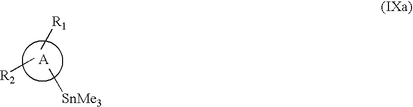

in which A, R₁ and R₂ are as defined in the formula (I),

either in the presence of triphenylphosphinearsenic and of a palladium catalyst, in the case where P₁ in the compounds of formula (XX) represents a triflate group,

or in the presence of a cupric halide compound, such as CuBr₂, and of a palladium catalyst, under polar solvent conditions, in the case where P₁ in the compounds of formula (XX) represents a halogen atom,

to also yield to the compounds of formula (I/g):

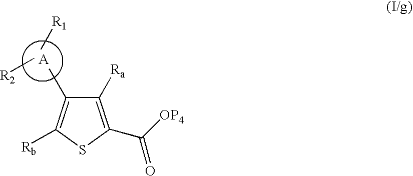

in which Rₐ, R_{b}, R₁, R₂, A and P₄ are as defined above,

which compounds of formula (I/g), in the specific case where they represent compounds of formula (I/h), in which A represents a phenyl group, R₂ represents a hydrogen atom, R₁ represents a hydroxyl group or a halogen atom (Hal) and Rₐ, R_{b} and P₄ have the same meaning as in the formula (I):

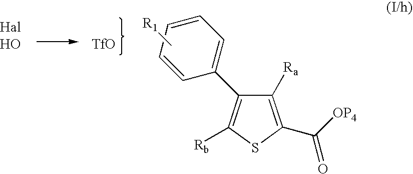

compounds of formula (I/h) which can be treated beforehand with trifluoromethanesulphonic anhydride in the presence of a strong base, in the case where R₁ represents a hydroxyl group, to produce the triflate activated derivative,

the said compounds, carrying an R₁ taking the definition halogen or triflate, could then:

either to be reacted, under basic conditions and in the presence of a palladium catalyst, with a compound of formula (X):

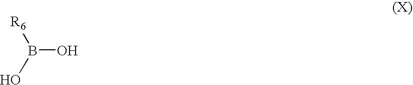

in which R₆ is as defined in the formula (I), that is to say that it represents a group selected from aryl and cycloalkyl and a heterocycle, each optionally being substituted,

to yield to the compounds of formula (I/i), a particular case of the compounds of formula (I):

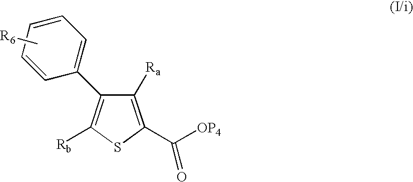

in which R₆, Rₐ, R_{b} and P₄ are as defined above,

or to be treated with bis(pinacolato)diborane, followed by an oxidation reaction, to yield to the compounds of formula (XXIII):

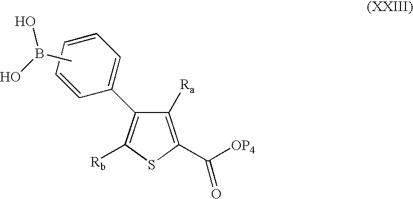

in which Rₐ, R_{b} and P₄ are as defined above,

which compounds of formula (XXIII) are reacted, under basic conditions and in the presence of a palladium catalyst, with a compound of formula (Xa):

R₆—P₂ (Xa)

in which R₆ is as defined in the formula (I), that is to say that it represents a group selected from aryl and cycloalkyl and a heterocycle, each optionally being substituted, and P₂ represents a halogen atom or a triflate group,

to also yield to the compounds of formula (I/i) as defined above:

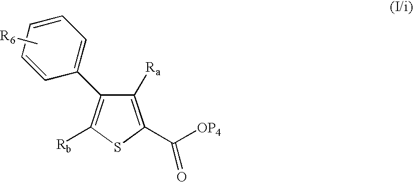

in which R₆, Rₐ, R_{b} and P₄ are as defined above,

or to be treated with hexamethylditin, in the presence of a palladium catalyst, to yield to the compounds of formula (XXIV):

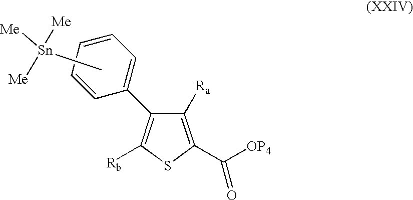

in which Rₐ, R_{b} and P₄ are as defined above,

which compounds of formula (XXIV) are reacted with a compound of formula (Xa) as defined above:

R₆—P₂ (Xa)

in which R₆ is as defined in the formula (I) and P₂ represents a halogen atom or a triflate group,

either in the presence of triphenylphosphinearsenic and of a palladium catalyst, in the case where P₂ represents a triflate group,

or in the presence of a cupric halide compound, such as CuBr₂, and of a palladium catalyst, under polar solvent conditions, in the case where P₂ represents a halogen atom,

to also yield to the compounds of formula (I/i) as defined above:

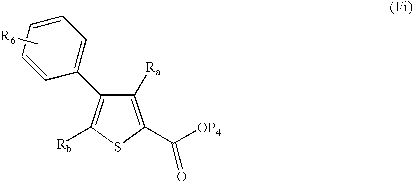

in which R₆, Rₐ, R_{b} and P₄ are as defined above,

or to be reacted with a compound of formula (Xb) (the said compounds of formula (Xb) being obtained by treatment of the compounds of formula (Xa) as defined above with hexamethylditin in the presence of a palladium catalyst):

R₆—SnMe₃ (Xb)

in which R₆ is as defined above,

either in the presence of triphenylphosphinearsenic and of a palladium catalyst, in the case where the compounds of formula (I/h) comprise a triflate group,

or in the presence of a cupric halide compound, such as CuBr₂, and of a palladium catalyst, under polar solvent conditions, in the case where the compounds of formula (I/h) comprise a halogen atom,

to also yield to the compounds of formula (I/i) as described above,

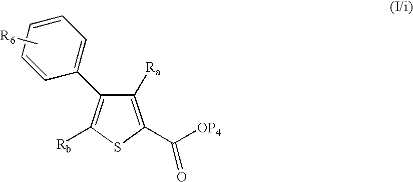

in which R₆, Rₐ, R_{b} and P₄ are as defined above,

the compounds of formulae (I/g), (I/h) and (I/i) together forming the compounds of formula (I/j):

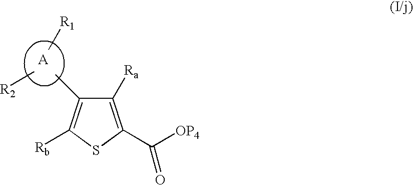

in which Rₐ, R_{b}, R₁, R₂, A and P₄ are as defined above,

which compounds of formula (I/j) are saponified under basic hydrolysis conditions to yield to the compounds of formula (I/k), a particular case of the compounds of formula (I):

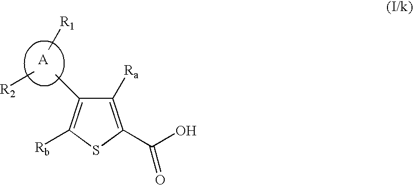

in which Rₐ, R_{b}, R₁, R₂ and A are as defined in the formula (I),

which compounds of formula (I/k) are either converted beforehand to the corresponding acid chloride by reaction with oxalyl chloride or treated directly, under peptide coupling conditions in the presence, for example, of a coupling agent and in a basic medium, with a compound of formula (V):

HY—R₃ (V)

in which Y and R₃ have the same meanings as in the compounds of formula (I),

to yield to the compounds of formula (I/l), a particular case of the compounds of formula (I):

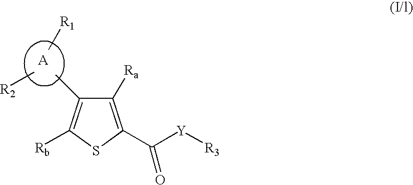

in which A, R₁, R₂, Rₐ, R_{b}, R₃ and Y are as defined above,

which compounds of formula (I/l) can be treated, for example with 2,4-bis(4-methoxyphenyl)-1,3-dithia-2,4-diphosphetane-2,4-disulphide, to yield to the compounds of formula (I/m), a particular case of the compounds of formula (I):

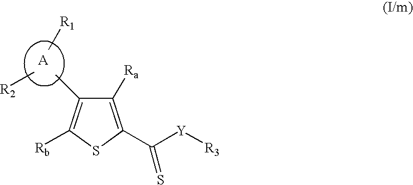

in which Rₐ, R_{b}, R₁, R₂, A, Y and R₃ are as defined in the formula (I),

the compounds (I/g) to (I/m) together representing few compounds of the invention, which are purified, if appropriate, according to a conventional purification technique, which can, if desired, be separated into their various isomers according to a conventional separating technique, and which are converted, if appropriate, into their addition salts with a pharmaceutically acceptable acid or base.

The compounds of the present invention, because of their pharmacological properties of inhibiting MMP-12, are useful for the prevention and treatment of respiratory pathologies, such as chronic obstructive pulmonary disease (COPD), emphysema, chronic bronchitis, chronic pulmonary inflammation, asthma, mucoviscidosis, acute respiratory distress syndrome (ARDS), respiratory allergies, including allergic rhinitis, and diseases related to the production of TNFα, including severe fibrotic pulmonary diseases, pulmonary sarcoidosis and silicosis. The compounds of the present invention also show, at a lower level, an inhibitory activity for metalloproteinase-13 (MMP-13), rendering them potentially useful for the treatment of pathologies involving this enzyme, such as cancer, osteoporosis, osteoarthritis, arthritis, rheumatoid arthritis, atherosclerosis, multiple sclerosis or cardiac insufficiency.

Advantageously, the compounds of the present invention are useful for the prevention and treatment of chronic obstructive pulmonary disease, of emphysema and of chronic bronchitis.

More particularly, the compounds of the present invention are useful for the treatment of smoking-related emphysema.

According to an alternative form of the invention, the compounds of formula (I) are useful for the prevention and treatment of asthma.

Another subject-matter of the present invention is pharmaceutical compositions including, as active principle, at least one compound of formula (I), one of its isomers or one of its addition salts with a pharmaceutically acceptable acid or base, alone or in combination with one or more pharmaceutically acceptable, nontoxic, inert excipients or vehicles.

Mention will more particularly be made, among the pharmaceutical compositions according to the invention, of those which are suitable for oral, parenteral (intravenous, intramuscular or subcutaneous), per- or transcutaneous, intravaginal, rectal, nasal, perlingual or respiratory administration.

The pharmaceutical compositions according to the invention for parenteral injections comprise in particular dispersions, suspensions, emulsions or sterile aqueous and nonaqueous solutions, as well as sterile powders for the reconstitution of the injectable solutions or dispersions.

The pharmaceutical compositions according to the invention, for solid oral administrations, comprise in particular simple or sugar-coated tablets, sublingual tablets, sachets, hard gelatin capsules or granules and, for oral, nasal or buccal liquid administrations, comprise in particular emulsions, solutions, suspensions, drops, syrups and aerosols.

The pharmaceutical compositions according to the invention, for administrations by the respiratory route, comprise in particular compositions in the form of solutions for aerosols or of powders for inhalers. When the compositions are aerosols, for the use of liquid aerosols, the compositions can be sterile stable solutions or can be solid compositions dissolved at the time useful for sterile apyrogenic water, in physiological saline or in any other pharmaceutically acceptable vehicle. For use in the form of dry aerosols intended to be directly inhaled, the active principle is optionally finely divided or micronized and used in combination with a water-soluble solid inert diluent or vehicle.

The pharmaceutical compositions for rectal administration are preferably suppositories and those for per- or transcutaneous administration comprise in particular powders, aerosols, creams, ointments, gels and patches.

The abovementioned pharmaceutical compositions illustrate the invention but do not limit it in any way.

Mention may be made, among pharmaceutically acceptable, nontoxic, inert excipients or vehicles, by way of indication and without implied limitation, of diluents, solvents, preservatives, wetting agents, emulsifiers, dispersing agents, binders, swelling agents, disintegrating agents, delayed-release agents, lubricants, absorbents, suspending agents, colorants, flavouring agents, and the like.

The useful dosage varies according to the age and the weight of the patient, the administration route, the pharmaceutical composition used, the nature and the seriousness of the complaint, and whether or not associated treatments are being taken. The dosage ranges from 1 mg to 1000 mg, taken on one or more occasion's daily.

The following examples illustrate the invention but do not limit it in any way.

The starting materials used are commercially available products or products prepared according to known procedures from commercially available compounds or compounds known to a person skilled in the art. The various preparations yield to synthetic intermediates useful for the preparation of the compounds of the invention.

The structures of the compounds described in the examples and in the preparations were determined according to conventional spectrophotometric techniques (infrared (IR), nuclear magnetic resonance (NMR), mass spectrometry (MS), including electrospray (ES) mass spectrometry, and the like) and the purity was determined by high performance liquid chromatography (HPLC).

### Preparation 1: 4-Bromo-N-(2-morpholin-4-ylethyl)thiophene-2-carboxamide

### Stage 1: 4-Bromothiophene-2-carboxylic acid

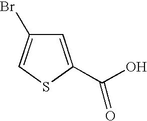

35.56 g of silver nitrate (4 equivalents) are added to a solution of 10 g of 4-bromothiophene-2-carboxaldehyde in 280 ml of ethanol, and 418 ml of 1 mol/l sodium hydroxide (8 equivalents) are added. The reaction medium is stirred at 40° C. for 3 hours, then filtered through celite and concentrated under reduced pressure. The white solid obtained is dissolved in water (300 ml). The aqueous phase is taken up in ethyl acetate (2×200 ml), acidified to pH=1 with a 1.0M hydrochloric acid solution and then extracted with ethyl acetate (2×250 ml). The organic phase is then dried over sodium sulphate, filtered and then concentrated under reduced pressure, making it possible to obtain 9.62 g of a white powder corresponding to the expected product.

Yield: 93% MS: MH⁻ 206

### Stage 2: 4-Bromo-N-(2-morpholin-4-ylethyl)thiophene-2-carboxamide

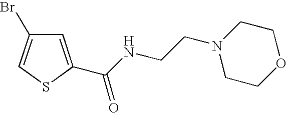

69.7 ml (3 equivalents) of oxalyl chloride are added dropwise to a solution of 9.62 g of the compound obtained in Stage 1 in 150 ml of anhydrous dichloromethane to which a few drops of dimethylformamide have been added. After reacting for 1 hour at ambient temperature and under nitrogen, the reaction medium is concentrated under reduced pressure, making it possible to obtain yellow oil, which is immediately dissolved in 150 ml of anhydrous dichloromethane. The reaction medium is cooled to 0° C. and then 6.4 ml of 2-morpholin-4-ylethanamine (1.05 equivalents) are added dropwise. The reaction medium is stirred at 0° C. for 1 hour and then 7.1 ml of triethylamine are added. The reaction medium is subsequently diluted with 500 ml of dichloromethane, washed with water (3×250 ml), dried over sodium sulphate, filtered and then concentrated under reduced pressure. Chromatography of the residue on silica gel (dichloromethane/methanol: 96/4) makes it possible to isolate 14.52 g of the expected product.

Yield: 98% ¹H NMR (CDCl₃) δ (ppm): 2.50 (m, 4H), 2.60 (m, 2H), 3.55 (m, 2H), 3.75 (m, 4H), 6.60 (bs, 1H), 7.35 (s, 1H), 7.40 (s, 1H) MS: MH⁺ 320

### Preparation 2: N-(2-Morpholin-4-ylethyl)-4-(trimethylstannyl)thiophene-2-carboxamide

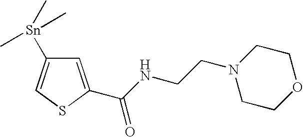

0.905 g of tetrakis(triphenylphosphine)palladium(0) (0.05 equivalent) and then 6.16 g of hexamethylditin (1.2 equivalents) are added to a solution, under an inert atmosphere, of 5.0 g of the compound of Preparation 1 in 80 ml of degassed ethylene glycol dimethyl ether. The reaction medium is stirred for 3 hours at 80° C., then diluted with ethyl acetate (200 ml), washed with water (3×100 ml), dried over sodium sulphate, filtered and then concentrated under cold conditions under reduced pressure. Chromatography of the residue on silica gel (dichloromethane/methanol: 98/2) makes it possible to isolate 3.91 g of the expected product.

Yield: 60% ¹H NMR (CDCl₃) δ (ppm): 0.25 (s, 9H), 2.45 (m, 4H), 2.55 (m, 2H), 3.45 (m, 2H), 3.65 (m, 4H), 6.50 (bs, 1H), 7.37 (s, 1H), 7.50 (s, 1H)

### Preparation 3: Ethyl 4-({[4-(trimethylstannyl)thien-2-yl]carboxamido}methyl)-cyclohexanecarboxylate

### Stage 1: Ethyl 4-{[(4-bromothien-2-yl)carboxamido]methyl}cyclohexanecarboxylate

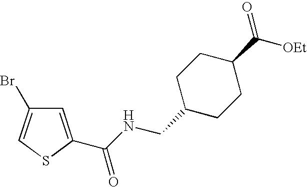

8.09 g of the product described in Preparation 10 and, dropwise, 10.16 ml (2.0 equivalents) of triethylamine are added to a solution of 7.56 g of the compound obtained in Stage 1 of Preparation 1 in 150 ml of dichloromethane. The reaction medium is stirred for 17 hours at ambient temperature, then taken up in dichloromethane and washed successively with water, with 1N hydrochloric acid, with a saturated sodium hydrogencarbonate solution and with a saturated sodium chloride solution. The product is dried over sodium sulphate, filtered and concentrated under reduced pressure. Chromatography of the residue on silica gel (dichloromethane/ethyl acetate: 9/1) makes it possible to isolate 6.5 g of the expected product.

Yield: 50% ¹H NMR (CDCl₃) δ (ppm): 7.40 (s, 1H), 7.38 (s, 1H), 6.30 (bs, 1H), 4.10 (q, 2H), 3.30 (m, 2H), 2.20 (m, 1H), 2.0 (dd, 2H), 1.90 (dd, 2H), 1.6 (m, 1H), 1.40 (q, 2H), 1.25 (t, 3H), 1.0 (q, 2H)

### Stage 2: Ethyl 4-({[4-(trimethylstannyl)thien-2-yl]carboxamido}methyl)cyclohexane-carboxylate

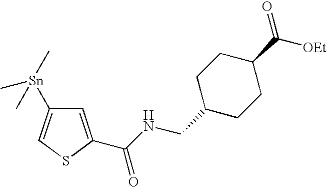

The product (0.367 g) is obtained according to the process of Preparation 2, using the product obtained in the preceding Stage 1 as substrate.

Yield: 74% ¹H NMR (CDCl₃) δ (ppm): 0.325 (s, 9H), 1.05 (q, 2H), 1.25 (t, 3H), 1.42 (q, 2H), 1.57 (bs, 1H), 1.90 (d, 2H), 2.05 (d, 2H), 2.25 (t, 1H), 3.30 (t, 2H), 4.12 (q, 2H), 6.00 (bs, 1H), 7.42 (s, 1H), 7.55 (s, 1H)

### Preparation 4: 4-Phenylcyclohex-1-enyl trifluoromethanesulphonate

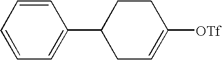

0.579 ml of trifluoromethanesulphonic anhydride (1.2 equivalents) is added dropwise to a solution of 0.5 g of 4-phenylcyclohexanone and 0.648 g of 2,6-di(tert-butyl)-4-methylpyridine (1.1 equivalents) in 8.0 ml of anhydrous dichloromethane. The reaction medium is subsequently stirred for 1 hour at 40° C., diluted with 25 ml of dichloromethane, washed with water (2×15 ml), dried over sodium sulphate, filtered and then concentrated under reduced pressure. Chromatography of the residue on silica gel (cyclohexane/ethyl acetate: 80/20) makes it possible to isolate 0.788 g of the expected product.

Yield: 89% ¹H NMR (CDCl₃) δ (ppm): 1.92 (m, 1H), 2.05 (m, 1H), 2.35 (m, 4H), 2.80 (m, 1H), 5.80 (m, 1H), 7.15 (m, 3H), 7.22 (m, 2H)

### Preparation 5: 4-[4-(Trifluoromethoxy)phenyl]thiophene-2-carbonyl chloride

### Stage 1: 4-[4-(Trifluoromethoxy)phenyl]thiophene-2-carbaldehyde

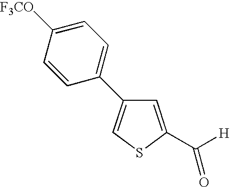

84.9 ml (2.1 equivalents) of a 2.0M potassium phosphate solution and 2.8 g (0.03 equivalent) of tetrakis(triphenylphosphine)palladium(0) are added to a solution of 12.3 g of 4-bromothiophene-2-carbaldehyde and 20.0 g of [4-(trifluoromethoxy)-phenyl]boronic acid (1.2 equivalents) in 70 ml of degassed DME. The reaction medium is stirred at 80° C. for 3 hours and is then concentrated under reduced pressure. The residue obtained is taken up in ethyl acetate. The solution is then filtered through celite, washed with water, dried over sodium sulphate, filtered and then concentrated under reduced pressure. Chromatography of the residue on silica gel (cyclohexane/ethyl acetate: 9/1) makes it possible to isolate 15.05 g of the expected product.

Yield: 68% ¹H NMR (CDCl₃) δ (ppm): 10.0 (s, 1H), 8.0 (s, 1H), 7.80 (s, 1H), 7.55 (m, 2H), 7.25 (m, 2H)

### Stage 2: 4-[4-(Trifluoromethoxy)phenyl]thiophene-2-carboxylic acid

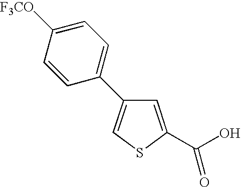

37.6 g (4 equivalents) of silver nitrate and 44.2 ml (8 equivalents) of a 1.0M aqueous sodium hydroxide solution are added to a solution of 15.05 g of the compound obtained in Stage 1 in 200 ml of ethanol. The reaction medium is stirred at 40° C. for 2 hours and is then filtered through celite and concentrate under reduced pressure. The aqueous phase is washed with a 1.0M aqueous hydrochloric acid solution and extracted with ethyl acetate, and the organic extract is dried over sodium sulphate, filtered and concentrated under reduced pressure, making it possible to obtain 15.514 g of a beige powder corresponding to the expected product.

Yield: 97.4% MS: MH⁻ 287

### Stage 3: 4-[4-(Trifluoromnethoxy)phenyl]thiophene-2-carbonyl chloride

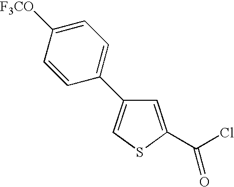

0.35 ml (2 equivalents) of oxalyl chloride is run onto a solution of 0.1 g of the compound obtained in Stage 2 in 5 ml of dichloromethane to which a few drops of dimethylformamide have been added. After reacting for 2 hours at ambient temperature, the reaction medium is concentrated under reduced pressure, making it possible to obtain yellow oil corresponding to the expected product, used in the crude form during the following preparations.

### Preparation 6: Ethyl 3-(6-aminopyridin-3-yl)propanoate

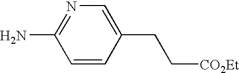

### Stage 1: Methyl 6-aminonicotinate

24.6 ml (3.4 equivalents) of 2.0M trimethylsilyldiazomethane in solution in cyclohexane are added to a solution of 2.0 g of 6-aminonicotinic acid in a chloroform/methanol (v/v: 10/30) mixture. The reaction medium is stirred at ambient temperature for 1 hour and is then hydrolysed with 100 μl of acetic acid and concentrated under reduced pressure. The crude product is triturated in a cyclohexane/ethyl acetate (1/2) mixture and filtered to produce 1.459 g of a yellow powder corresponding to the expected product.

Yield: 66% MS: MH⁺ 153

### Stage 2: Methyl 6-[bis(tert-butoxycarbonyl)amino]nicotinate

4.18 g of di(tert-butyl) dicarbonate (2.0 equivalents) and 0.117 g of dimethylaminopyridine (0.1 equivalent) are added at 0° C. to a solution of 1.459 g of the product obtained in Stage 1 in 50 ml of dichloromethane and then 2.7 ml (2.0 equivalents) of triethylamine are added dropwise. The reaction medium is stirred at ambient temperature for 4 hours, concentrated under reduced pressure, washed with water and then washed with a saturated aqueous sodium chloride solution. The reaction medium is extracted with ethyl acetate and dried over sodium sulphate, then filtered and concentrated under reduced pressure. Chromatography of the residue on silica gel (dichloromethane/methanol: 9/1) makes it possible to isolate 1.875 g of the expected product.

Yield: 55% MS: MH⁺ 353

### Stage 3: Di(tert-butyl) 5-(hydroxymethyl)pyridin-2-ylimidodicarbonate

0.159 g of lithium aluminium hydride (1.5 equivalents) is added at 0° C. to a solution of 0.985 g of the compound obtained in Stage 2 in 10 ml of tetrahydrofuran. The reaction medium is stirred at ambient temperature for 17 hours, is then hydrolysed with a saturated ammonium chloride solution and is extracted with ethyl acetate. The solution is washed with water, dried over sodium sulphate, then filtered and concentrated under reduced pressure. Chromatography of the residue on silica gel (dichloromethane/methanol: 95/5) makes it possible to isolate 1.543 g of the expected product.

Yield: 89% MS: MH⁺ 325

### Stage 4: Di(tert-butyl) 5-formylpyridin-2-ylimidodicarbonate

0.686 g of 1,1,1-tri(acetyloxy)-1,1-dihydro-1,2-benziodoxol-3(1H)-one (1.5 equivalents) is added at 0° C. to a solution of 0.894 g of the compound obtained in Stage 3 in 10 ml of dichloromethane. The reaction medium is stirred at ambient temperature for 2 hours, then hydrolysed with water and extracted with dichloromethane. The organic phase is washed with water, dried over sodium sulphate, then filtered and concentrated under reduced pressure. Chromatography of the residue on silica gel (cyclohexane/ethyl acetate: 80/20) makes it possible to isolate 0.456 g of the expected product.

Yield: 51% MS: MH⁺ 355

### Stage 5: (2E)-3-{6-[Bis(tert-butoxycarbonyl)amino]pyridin-3-yl}propen-2-oic acid

0.177 g of malonic acid (1.2 equivalents) is added to a solution of 0.456 g of the compound obtained in Stage 4 in 0.3 ml of pyridine and 2.0 ml of ethanol. The reaction medium is stirred at reflux for 17 hours. The precipitate obtained is filtered off and washed with ethyl acetate to isolate 0.16 g of a white powder corresponding to the expected product.

Yield: 31% MS: MH⁺ 365, MH⁻ 363

### Stage 6: 3-(6-[Bis(tert-butoxycarbonyl)amino]pyridin-3-yl)propanoic acid

27.6 mg of palladium-on-charcoal (0.1 equivalent) are added to a solution of 0.16 g of the compound obtained in Stage 5 in 3 ml of ethanol. The reaction medium is stirred at 40° C. for 4 hours under 5 bar of hydrogen. The solution is then filtered through celite and concentrated under reduced pressure to produce 0.133 g of a white powder corresponding to the expected product.

Yield: 83% MS: MH⁻ 365

### Stage 7: Ethyl 3-(6-aminopyridin-3-yl)propanoate

0.4 ml of concentrated sulphuric acid (10 volumes) is added to a solution of 0.133 g of the compound obtained in Stage 6 in 3 ml of ethanol. The reaction medium is stirred at reflux for 17 hours. The solution is then concentrated under reduced pressure and basified with a saturated sodium hydrogencarbonate solution to pH 8. The solution obtained is extracted with ethyl acetate and the organic phase is dried over sodium sulphate and then filtered to produce, after concentrating under reduced pressure, 0.041 g of the expected product.

Yield: 59% MS: MH⁺ 195, MH⁻ 193

### Preparation 7: Ethyl (2E)-3-(4-aminophenyl)acrylate

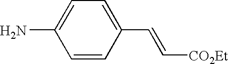

The product (0.153 g) is obtained according to the process of Stage 7 of Preparation 6, using 4-aminocinnamic acid hydrochloride as substrate.

Yield: 32% MS: MH⁺ 192

### Preparation 8: Diethyl 4-(aminomethyl)phthalate

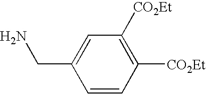

### Stage 1: Diethyl 4-bromophthalate

1.0 ml of concentrated sulphuric acid is added to a solution of 1.0 g of 4-bromophthalic acid in 20 ml of ethanol. The reaction medium is irradiated at 79° C. with microwave radiation (power 50 W) for 30 minutes. The reaction medium is concentrated under reduced pressure and extracted with ethyl acetate. The solution is washed with water and dried over sodium sulphate, then filtered to produce, after concentrating under reduced pressure, 1.08 g of the expected product.

Yield: 88%

### Stage 2: Diethyl 4-cyanophthalate

0.508 g (1.8 equivalents) of copper cyanide is added to a solution of 0.95 g of the compound obtained in Stage 1 in 10 ml of N-methyl-2-pyrrolidinone. The reaction medium is stirred at 200° C. for 17 hours. The solution is then hydrolysed with an aqueous ammonium hydroxide solution and extracted with ethyl acetate. The solution is washed with water, dried over sodium sulphate and then filtered to produce, after evaporating under reduced pressure, 1.2 g of brown oil. Chromatography of the residue on silica gel (cyclohexane/ethyl acetate: 8/2) makes it possible to isolate 0.320 g of the expected product.

Yield: 41% MS: MH⁺ 273

### Stage 3: Diethyl 4-(aminomethyl)phthalate

0.5 ml of 36% hydrochloric acid and 15 mg (0.1 equivalent) of 10% palladium-on-charcoal are added to a solution of 0.15 g of the product obtained in Stage 2 in 1 ml of ethanol. The reaction medium is stirred under 15 bar of hydrogen at 60° C. for 2 hours. The medium is filtered through celite and concentrated under reduced pressure to produce 0.3 g of a beige powder.

Yield: 24% MS: MH⁺ 252

### Preparation 9: Ethyl 3-(4-aminophenyl)propanoate

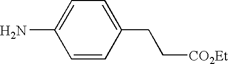

The product (0.503 g) is obtained according to the process of Stage 7 of Preparation 6, using 3-(4-aminophenyl)propionic acid as substrate.

Yield: 86% ¹H NMR (CDCl₃) δ (ppm): 7.0 (m, 2H), 6.6 (m, 2H), 4.1 (q, 2H), 2.8 (q, 2H), 1.25 (t, 3H)

### Preparation 10: Ethyl trans-4-(aminomethyl)cyclohexanecarboxylate

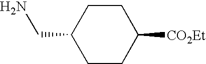

0.4 ml of sulphuric acid is added to a solution of 0.2 g of trans-4-(aminomethyl)cyclohexanecarboxylic acid in 5 ml of ethanol. The reaction medium is brought to reflux for 17 hours and then concentrated under reduced pressure. The residue is taken up in ethyl acetate. The solution is basified to pH=9 by addition of a 1.0M aqueous sodium hydroxide solution, washed with water, dried over sodium sulphate then concentrated under reduced pressure, making it possible to obtain 0.129 g of a yellow oil, corresponding to the expected product.

Yield: 55% MS: MH⁺ 186

### Preparation 11: Ethyl [4-(aminomethyl)phenyl]acetate

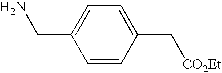

### Stage 1: Ethyl (4-bromophenyl)acetate

The product (4.208 g) is obtained according to the process of Stage 7 of Preparation 6, using (4-bromophenyl)acetic acid as substrate.

Yield: 93% MS: MH⁺ 243, MH⁻ 242

### Stage 2: Ethyl (4-cyanophenyl)acetate

The product (0.2 g) is obtained according to the process of Stage 2 of Preparation 8, using the product obtained in the preceding Stage 1 as substrate.

Yield: 52% ¹H NMR (CDCl₃) δ (ppm): 7.6 (m, 2H), 7.5 (m, 1H), 7.4 (m, 1H), 3.8 (s, 3H), 3.7 (s, 2H)

### Stage 3: Ethyl [4-(aminomethyl)phenyl]acetate

The product (0.045 g) is obtained according to the process of Stage 3 of Preparation 8, using the product obtained in the preceding Stage 2 as substrate.

Yield: 17% MS: MH⁻ 223

### Preparation 12: Ethyl 3-(aminomethyl)benzoate

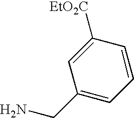

### Stage 1: 3-(Aminomethyl)benzoic acid

The product (0.213 g) is obtained according to the process of Stage 3 of Preparation 8, using 3-cyanobenzoic acid as substrate.

Yield: 100% MS: MH⁺ 152

### Stage 2: Ethyl 3-(aminomethyl)benzoate

The product (0.15 g) is obtained according to the process of Stage 7 of Preparation 6, using the product obtained in the preceding Stage 1 as substrate.

Yield: 60% MS: MH⁺ 180

### Preparation 13: Ethyl [3-(aminomethyl)phenyl]acetate

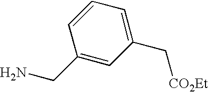

### Stage 1: (3-Iodophenyl)acetic acid

A solution of 0.2 g of (3-iodophenyl)acetonitrile in 1.0 ml of a 1.0M aqueous sodium hydroxide solution is brought to reflux for 4 hours. The solution is extracted with diethyl ether and the aqueous phase is acidified with a 1.0M hydrochloric acid solution. The solution derived from extracting with diethyl ether is washed with water, dried over sodium sulphate, filtered and then evaporated under reduced pressure to produce 0.17 g of the expected product.

Yield: 83% ¹H NMR (DMSO) δ (ppm): 7.65 (s, 1H), 7.5 (d, 1H), 7.3 (d, 1H), 7.1 (m, 1H), 3.6 (s, 2H)

### Stage 2: Ethyl (3-iodophenyl)acetate

The product (0.164 g) is obtained according to the process of Stage 7 of Preparation 6, using the product obtained in the preceding Stage 1 as substrate.

Yield: 82% ¹H NMR (CDCl₃) δ (ppm): 7.65 (s, 1H), 7.6 (d, 1H), 7.3 (s, 1H), 7.1 (m, 1H), 4.2 (q, 2H), 3.5 (s, 2H), 1.25 (t, 3H)

### Stage 3: Ethyl (3-cyanophenyl)acetate

The product (0.065 g) is obtained according to the process of Stage 2 of Preparation 8, using the product obtained in the preceding Stage 2 as substrate.

Yield: 61% MS: MH⁻ 188

### Stage 4: Ethyl [3-(aminomethyl)phenyl]acetate

The product (0.071 g) is obtained according to the process of Stage 3 of Preparation 8, using the product obtained the preceding Stage 3 as substrate.

Yield: 100% MS: MH⁺ 194

### Preparation 14: Ethyl 4-(aminomethyl)cyclohexanecarboxylate

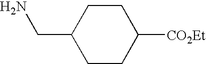

The product (0.239 g) is obtained according to the process of Stage 7 of Preparation 6, using 4-(aminomethyl)cyclohexanecarboxylic acid as substrate.

Yield: 81 % MS: MH⁺ 186

### Preparation 15: Methyl 6-(bromomethyl)nicotinate

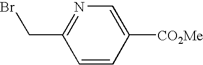

### Stage 1: Methyl 6-methylnicotinate

The product (0.708 g) is obtained according to the process of Stage 1 of Preparation 6, using 6-methylnicotinic acid as substrate.

Yield: 33% MS: MH⁺ 152

### Stage 2: Methyl 6-(bromomethyl)nicotinate

0.471 g (1.0 equivalent) of N-bromosuccinimide and 64 mg (0.1 equivalent) of benzoyl peroxide are added to a solution of 0.4 g of the product obtained in the preceding Stage 1 in 15 ml of carbon tetrachloride. The solution is brought to reflux for 6 hours, then filtered and evaporated under reduced pressure. The residue is purified by chromatography on silica gel (dichloromethane/ethyl acetate: 95/5) to produce 0.262 g of the expected product.

Yield: 43% MS: MH⁺ 231

### Preparation 16: Ethyl 4-(aminomethyl)benzoate

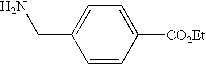

The product (1.03 g) is obtained according to the process of Stage 7 of Preparation 6, using 4-(aminomethyl)benzoic acid as substrate.

Yield: 87% MS: MH⁺ 179

### Preparation 17: Ethyl 4-{1[(4-bromothien-2-yl)carboxamido]methyl}benzoate

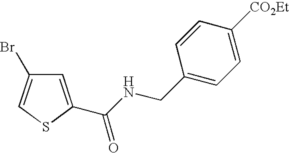

### Stage 1: 4-Bromothiophene-2-carbonyl chloride

The product (4.06 g) is obtained according to the process of Stage 3 of Preparation 5, using 4-bromothiophene-2-carboxylic acid as substrate.

### Stage 2: Ethyl 4-{[(4-bromothien-2-yl)carboxamido]methyl}benzoate

0.700 g (1.05 equivalents) of the product obtained in Preparation 16 and 0.77 ml of triethylamine (1.5 equivalents) are added to a solution of 0.833 g of the product obtained in the preceding Stage 1 in 15 ml of dichloromethane. The reaction medium is stirred at ambient temperature for 17 hours. The solution is hydrolysed with water and washed with a 1.0M aqueous hydrochloric acid solution and then with a saturated sodium hydrogencarbonate solution. The organic phase is dried over sodium sulphate, filtered and concentrated under reduced pressure. The residue is purified by chromatography on silica gel (dichloromethane/ethyl acetate: 97/3) to produce 0.1 g of the expected product.

Yield: 7% ¹H NMR (CDCl₃) δ (ppm): 8.0 (m, 2H), 7.4 (m, 4H), 6.4 (bs, 1H), 4.6 (d, 2H), 4.3 (q, 2H), 1.40 (t, 3H)

### Preparation 18: Ethyl {4-[(4-bromothien-2-yl)carboxamido]phenyl}acetate

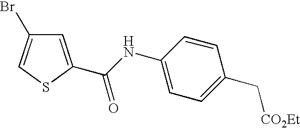

### Stage 1: Ethyl (4-aminophenyl)acetate

The product (1.686 g) is obtained according to the process of Stage 7 of Preparation 6 using 4-aminophenylacetic acid as substrate.

Yield: 71% MS: MH⁺ 179

### Stage 2: Ethyl {4-[(4-bromothien-2-yl)carboxamido]phenyl}acetate

The product (0.550 g) is obtained according to the process of Stage 2 of Preparation 17, using the product obtained in Stage 1 of Preparation 17 as substrate, and the product described in the preceding Stage 1 as cosubstrate.

Yield: 32% MS: MH⁺ 369

### Preparation 19: 4-(4-Methoxyphenyl)thiophene-2-carbonyl chloride

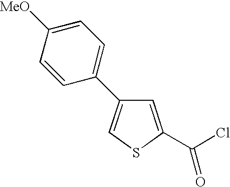

### Stage 1: 4-(4-Methoxyphenyl)thiophene-2-carbaldehyde

28.8 ml (2.1 equivalents) of potassium phosphate and 0.95 g (0.03 equivalent) of tetrakis(triphenylphosphine)palladium(0) are added to a solution of 5.24 g of 4-bromothiophene-2-carbaldehyde and 5.0 g of 4-(methoxy)phenylboronic acid (1.2 equivalents) in 50 ml of degassed DME. The reaction medium is stirred at 80° C. for 3 hours and then concentrated under reduced pressure. The residue obtained is taken up in ethyl acetate. After filtering through celite and washing with water, the reaction medium is extracted with ethyl acetate. The organic phases are combined, dried over sodium sulphate, filtered and then concentrated under reduced pressure. Chromatography of the residue on silica gel (cyclohexane/ethyl acetate: 9/1) makes it possible to isolate the expected product (2.594 g).

Yield: 36% MS: MH⁺ 218

### Stage 2: 4-(4-Methoxyphenyl)thiophene-2-carboxylic acid

The product (2.34 g) is obtained according to the process of Stage 2 of Preparation 5, using the product obtrained in the preceding Stage 1 as substrate.

Yield: 84% MS: MH⁻ 232

### Stage 3: 4-(4-Methoxyphenyl)thiophene-2-carbonyl chloride

The product (0.384 g) is obtained according to the process of Stage 3 of Preparation 5, using the product obtained in the preceding Stage 2 as substrate.

### Preparation 20: Ethyl 3-[4-(aminomethyl)phenyl]propanoate

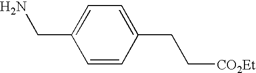

### Stage 1: Ethyl 3-(4-chlorophenyl)propanoate

The product (1.046 g) is obtained according to the process of Stage 7 of Preparation 6, using 3-(4-chlorophenyl)propanoic acid as substrate.

Yield:91 % MS: MH⁺ 276

### Stage 2: Ethyl 3-(4-cyanophenyl)propanoate

The product (0.076 g) is obtained according to the process of Stage 2 of Preparation 8, using the product obtained in the preceding Stage 1 as substrate.

Yield: 16% MS: MH⁺ 204

### Stage 3: Ethyl 3-[4-(aminomethyl)phenyl]propanoate

The product (0.131 g) is obtained according to the process of Stage 3 of Preparation 8, using the product obtained in the preceding Stage 2 as substrate.

Yield: 77% MS: MH⁺ 208

### Preparation 21: Ethyl 3-(3-aminophenyl)propanoate

### Stage 1: Ethyl 3-(3-bromophenyl)propanoate

The product (1.778 g) is obtained according to the process of Stage 7 of Preparation 6, using 3-(3-bromophenyl)propanoic acid as substrate.

Yield: 80% ¹H NMR (CDCl₃) δ (ppm): 7.35 (m, 2H), 7.1 (m, 2H), 4.1 (q, 2H), 2.9 (q, 2H), 2.6 (q, 2H), 1.20 (t, 3H)

### Stage 2: Ethyl 3-(3-cyanophenyl)propanoate

The product (1.277 g) is obtained according to the process of Stage 2 of Preparation 8, using the product obtained in the preceding Stage 1 as substrate.

Yield: 91% ¹H NMR (CDCl₃) δ (ppm): 7.55 (s, 2H), 7.4 (m, 1H), 7.35 (m, 1H), 4.1 (q, 2H), 2.9 (q, 2H), 2.6 (q, 2H), 1.20 (t, 3H)

### Stage 3: Ethyl 3-(3-aminophenyl)propanoate

The product (0.619 g) is obtained according to the process of Stage 3 of Preparation 8, using the product obtained in the preceding Stage 2 as substrate.

Yield: 40% MS: MH⁺ 208

### Preparation 22: 7-Ethyl aminoheptanoate

The product (0.470 g) is obtained according to the process of Stage 7 of Preparation 6, using 6-aminocaproic acid as substrate.

Yield: 78% MS: MH⁺ 160

### Preparation 23: Methyl trans-3-(3-aminocyclohexyl)propanoate

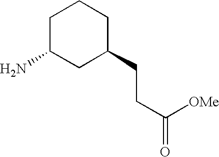

### Stage 1: trans-3-[3-(tert-Butoxycarbonylamino)cyclohexyl]propanoic acid

4.5 g of ruthenium hydroxide-on-charcoal are added to a solution of 20 g of 3-nitrocinnamic acid and of 4.9 g of lithium hydroxide monohydrate in 400 ml of water. The reaction medium is stirred at 110° C. for 48 hours under 140-150 bar of hydrogen. After removing the catalyst by filtration, the filtrate is diluted in 400 ml of tetrahydrofuran. A solution of 28.6 g of di(tert-butyl) dicarbonate in 400 ml of tetrahydrofuran is slowly added to the preceding solution. The mixture obtained is stirred for 18 hours at ambient temperature then concentrated under reduced pressure. The residual solution (500 ml) is brought to pH=6 by adding a saturated aqueous potassium dihydrogenphosphate solution. The precipitate obtained is extracted with ethyl acetate and the organic phase is concentrated under reduced pressure. The residue obtained is crystallized from ethyl acetate (40 ml) to give 7.5 g of the expected product.

Yield: 26.9%

### Stage 2: Methyl trans-3-[3-(tert-butoxycarbonylamino)cyclohexyl]propanoate

The product (0.51 g) is obtained according to the process of Stage 1 of Preparation 6, using 0.5 g of the product obtained in the preceding Stage 1 as substrate.

Yield: 97.4%

### Stage 3: Methyl trans-3-(3-aminocyclohexyl)propanoate

0.85 ml of trifluoroacetic acid is added at 0° C. to a solution of 315 mg of the product obtained in Stage 2 in 5 ml of anhydrous dichloromethane. The reaction medium is stirred for 17 hours at ambient temperature, washed with water (30 ml) and then with a saturated sodium hydrogencarbonate solution (30 ml), dried over sodium sulphate, filtered and concentrated under reduced pressure. Recrystallization of the residue from diisopropyl ether makes it possible to isolate 79 mg of the expected product.

Yield: 39% MS: MH⁺ 186

### Preparation 24: Methyl trans-3-aminocyclohexylacetate

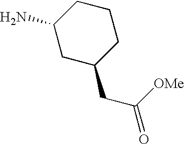

### Stage 1: trans-3-(tert-Butoxycarbonylamino)cyclohexylacetic acid

The product (8.9 g) is obtained according to the process of Stage 1 of Preparation 23, using 13.2 g of 3-aminophenylacetic acid as substrate.

Yield: 39.6%

### Stage 2: Methyl trans-3-(tert-butoxycarbonylamino)cyclohexylacetate

The product (0.54 g) is obtained according to the process of Stage 1 of Preparation 6, using 0.5 g of the product obtained in the preceding Stage 1 as substrate.

Yield: 100%

### Stage 3: Methyl trans-3-aminocyclohexylacetate

The product (0.204 g) is obtained according to the process of Stage 3 of Preparation 23, using the product obtained in the preceding Stage 2 as substrate.

Yield: 99% MS: MH⁺ 172

### Preparation 25: Methyl trans-3-(4-aminocyclohexyl)propanoate

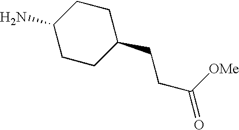

### Stage 1: trans-3-[4-(tert-Butoxycarbonylamino)cyclohexyl]propanoic acid

The product (6.3 g) is obtained according to the process of Stage 1 of Preparation 23, using 20 g of 4-nitrocinnamic acid as substrate.

Yield: 22.4%

### Stage 2: Methyl trans-3-[4-(tert-butoxycarbonylamino)cyclohexyl]propanoate

The product (0.52 g) is obtained according to the process of Stage 1 of Preparation 6, using 0.5 g of the product obtained in the preceding Stage 1 as substrate.

Yield: 99%

### Stage 3: Methyl trans-3-(4-aminocyclohexyl)propanoate

The product (0.201 g) is obtained according to the process of Stage 3 of Preparation 23, using the product obtained in the preceding Stage 2 as substrate.

Yield: 99% MS: MH⁺ 186

### Preparation 26: Methyl 4-(aminomethyl)cyclohexylacetate

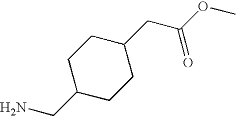

### Stage 1: 4-[(tert-Butoxycarbonylamino)methyl]cyclohexylacetic acid

The product (1.9 g) is obtained according to the process of Stage 1 of Preparation 23, using 3.99 g of 4-(aminomethyl)phenylacetic acid as substrate.

Yield: 40.7%

### Stage 2: Methyl 4-[(tert-butoxycarbonylamino)methyl]cyclohexylacetate

The product (0.508 g) is obtained according to the process of Stage 1 of Preparation 6, using 0.5 g of the product obtained in the preceding Stage 1 as substrate.

Yield: 96.4%

### Stage 3: Methyl 4-(aminomethyl)cyclohexylacetate

The product (0.216 g) is obtained according to the process of Stage 3 of Preparation 23, using the product obtained in the preceding Stage 2 as substrate.

Yield: 100% MS: MH⁺ 186

### Preparation 27: Ethyl 5-aminomethyl-2-hydroxybenzoate hydrochloride

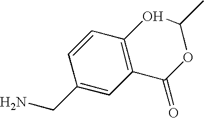

### Stage 1: Ethyl 5-iodo-2-hydroxybenzoate

The product (1.936 g) is obtained according to the process of Stage 7 of Preparation 6, using 5-iodo-2-hydroxybenzoic acid as substrate.

Yield: 87.5%

### Stage 2: Ethyl 5-aminomethyl-2-hydroxybenzoate hydrochloride

The product (0.52 g) is obtained according to the process of Stages 2 and 3 of Preparation 8, using the product obtained in the preceding Stage 1 as substrate.

Yield: 67%

### Preparation 28: Methyl 6-(aminomethyl)nicotinate bishydrochloride

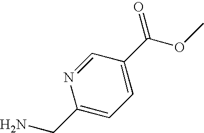

### Stage 1: Methyl 6-cyanonicotinate

803 ml (3.4 equivalents) of 2.0M trimethylsilyldiazomethane in solution in diethyl ether are added over a period of one hour to a solution of 70 g of methyl 6-aminonicotinic acid in a 1/1 diethyl ether/methanol mixture cooled to 0° C. The reaction mixture is stirred at ambient temperature for 1 hour and is then concentrated under reduced pressure. The orange solid residue obtained is dissolved in 1.4 l of ethyl acetate and washed successively with water and an aqueous sodium carbonate solution. After extracting the aqueous phases with ethyl acetate, the organic phases are combined, dried over magnesium sulphate, filtered and concentrated under reduced pressure to give 80.5 g of the expected product in the form of an orange solid.

Yield: 100% ¹H NMR (CDCl₃) δ (ppm): 9.29 (dd, 1H), 8.47 (dd, 1H), 7.84 (dd, 1H), 4.02 (s, 3H)

### Stage 2: Methyl 6-(aminomethyl)nicotinate bishydrochloride

75.6 ml of 36% hydrochloric acid and 7 g (0.1 equivalent by mass) of 10% palladium-on-charcoal are added to a solution of 70 g of the product obtained in the preceding Stage 1 in 1.4 l of ethanol. The reaction medium is stirred under 1 bar of hydrogen at ambient temperature overnight, filtered and washed with ethanol under a nitrogen atmosphere.

After evaporating the ethanol, the solid obtained is taken up in 1.4 l of ethanol and heated at 50° C. for one hour. The reaction medium is filtered while hot and the solid obtained is washed with warm ethanol (40° C.). After repeating this operation, the organic phases are combined and concentrated under reduced pressure to give 60.8 g of a green solid, successively purified by treatment with active charcoal and recrystallization from ethyl acetate.

Yield: 47% ¹H NMR (CDCl₃) δ (ppm): 9.07 (dd, 1H), 8.77 (br s, 4H), 8.33 (dd, 1H), 7.72 (dd, 1H), 4.28 (br s, 2H), 3.90 (s, 3H)

### Preparation 29: 4-(2,2,2-Trifluoroethoxy)phenylboronic acid

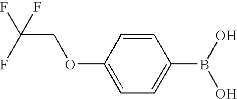

### Stage 1: 1-Bromo-4-(2,2,2-trifluoroethoxy)benzene

A solution of bromophenol (3 g) in hexamethylphosphoramide (17.3 ml) is added dropwise to sodium hydroxide (1 equivalent) washed beforehand with ether. The medium is stirred at ambient temperature until gas evolution has ceased. After addition of 2,2,2-trifluoroethyl methanesulphonate (1.2 equivalents), the solution is irradiated under microwave radiation at 140° C. at atmospheric pressure for 5 hours. The reaction medium is subsequently diluted with 150 ml of diethyl ether and washed with water. The organic phase is dried over sodium sulphate, filtered and then concentrated under reduced pressure. Chromatography of the residue on silica gel (cyclohexane 100%) makes it possible to isolate 1.28 g of the expected product.

Yield: 30% ¹H NMR (CDCl₃) δ (ppm): 7.40 (d, 2H), 6.81 (d, 2H), 4.32 (m, 2H)

### Stage 2: 4,4,5,5-Tetramethyl-2-[4-(2,2,2-trifluoroetboxy)phenyl]-1,3,2-dioxaborolane

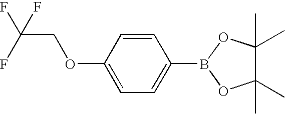

The product (642 mg) is obtained according to the process of Stage 4 of Example 18, using the compound obtained in the preceding Stage 1 as substrate.

Yield: 42% ¹H NMR (CDCl₃) δ (ppm): 7.76 (d, 2H), 6.90 (d, 2H), 4.39 (m, 2H), 1.32 (s, 1H)

### Stage 3: 4-(2,2,2-Trifluoroethoxy)phenylboronic acid

The product (54 mg) is obtained according to the process of Stage 5 of Example 18 using 0.1 g of the compound obtained in the preceding Stage 2 as substrate.

Yield: 74% ¹H NMR (DMSO) δ (ppm): 7.95 (s, 2H), 7.79 (d, 2H), 7.00 (d, 2H), 4.78 (m, 2H)

### Preparation 30: [4-(Methoxycarbonylmethyl)phenyl]boronic acid

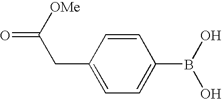

### Stage 1: Methyl (4-bromophenyl)acetate

The product (0.9 g) is obtained according to the process of Stage 7 of Preparation 6, using 4-bromophenylacetic acid as substrate.

Yield: 85% ¹H NMR (CDCl₃) δ (ppm): 7.39 (m, 2H), 7.18 (m, 2H), 3.70 (s, 3H), 3.58 (s, 2H) HPLC: 100%

### Stage 2: Methyl [4-(4,4,5,5-teframethyl-1,3,2-dioxaborolan-2-yl)phenyl]acetate

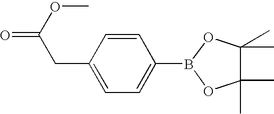

The product (0.44 g) is obtained according to the process of Stage 4 of Example 18, using 0.5 g of the compound obtained in the preceding Stage 1 as substrate.

Yield: 73% ¹H NMR (CDCl₃) δ (ppm): 7.79 (m, 2H), 7.25 (m, 2H), 3.70 (s, 3H), 3.64 (s, 2H), 1.32 (s, 12H)

### Stage 3: [4-(Methoxycarbonylmethyl)phenyl]boronic acid

The product (193 mg) is obtained according to the process of Stage 5 of Example 18, using the compound obtained in the preceding Stage 2 as substrate.

Yield: 63% ¹H NMR (DMSO) δ (ppm): 8.00 (s, 2H), 7.72 (d, 2H), 7.20 (d, 2H), 3.68 (s, 2H), 3.61 (s, 3H)

### Preparation 31: 2-Bromo-5-phenylpyridine

### Stage 1: 6-Bromo-3-(trimethylstannyl)pyridine

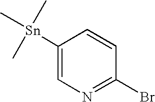

1.77 ml of a solution of n-butyllithium (1.0M) in tetrahydrofuran are added dropwise to a solution of 1 g of 2,5-dibromopyridine in anhydrous diethyl ether (28 ml) cooled to −78° C. The reaction medium is stirred at −78° C. for 4 hours and then 4.43 ml of a solution of trimethyltin chloride (2.5M) in hexane are added dropwise. The reaction medium is subsequently stirred at ambient temperature for 0.5 hour, diluted with 100 ml of diethyl ether and washed with water (3×50 ml). The organic phase is dried over sodium sulphate, filtered and then concentrated under reduced pressure. Chromatography of the residue on silica gel (cyclohexane/ethyl acetate: 95/5) makes it possible to isolate 0.505 g of the expected product.

Yield: 37% ¹H NMR (DMSO) δ (ppm): 8.35 (s, 1H), 7.80 (d, 1H), 7.55 (d, 1H), 0.30 (s, 9H)

### Stage 2: 2-Bromo-5-phenylpyridine

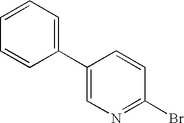

191 μl of iodobenzene (1.1 equivalents) and 53.9 mg of tetrakis(triphenylphosphine)palladium(0) (0.03 equivalent) are added to a solution of 0.5 g of the substrate obtained in the preceding Stage 1 in 5.0 ml of degassed DME. The reaction medium is stirred at 80° C. for 24 hours and is then diluted with ethyl acetate (75 ml). The organic phase is washed with water (3×30 ml), dried over sodium sulphate, filtered and then concentrated under reduced pressure. Chromatography of the residue on silica gel (dichloromethane: 100%) makes it possible to isolate 110.3 mg of the expected product.

Yield: 30% ¹H NMR (DMSO) δ (ppm): 8.70 (m, 1H), 8.05 (m, 1H), 7.75 (m, 3H), 7.50 (m, 3H)

### EXAMPLE 1

### 4-(4-Isopropylphenyl)-N-(2-morpholin-4-ylethyl)thiophene-2-carboxamide

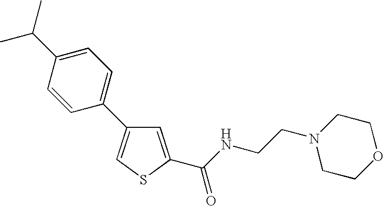

61.6 mg of (4-isopropylphenyl)boronic acid (1.2 equivalents), 10.8 mg of tetrakis(triphenylphosphine)palladium(0) (0.03 equivalent) and 0.328 ml of a 2.0M potassium phosphate solution (2.1 equivalents) are added to a solution under nitrogen of 100 mg of the compound of Preparation 1 in 3.0 ml of degassed DME. The reaction medium is subsequently stirred for 3 hours at 80° C., diluted with ethyl acetate (20 ml), washed with water (2×15 ml), dried over sodium sulphate, filtered and concentrated under reduced pressure. Chromatography of the residue on silica gel (dichloromethane/methanol: 98/2) makes it possible to isolate 51.4 mg of the expected product.

Yield: 46% ¹H NMR (CDCl₃) δ (ppm): 1.30 (d, 6H), 2.575 (m, 4H), 2.60 (m, 2H), 2.95 (m, 1H), 3.57 (m, 2H), 3.75 (m, 4H), 6.65 (bs, 1H), 7.30 (d, 2H), 7.50 (m, 3H), 7.75 (s, 1H) MS: MH⁺ 359 HPLC: 100%

### EXAMPLE 2

### 4-(4-Biphenyl)-N-(2-morpholin-4-ylethyl)thiophene-2carboxamide

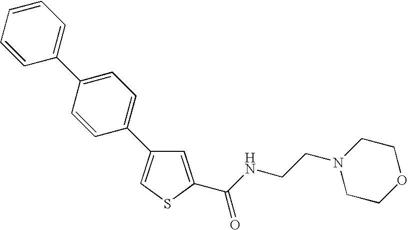

The product (57.7 mg) is obtained according to the process of Example 1, using (1,1′-biphenyl-4-yl)boronc acid as cosubstrate.

Yield: 47% ¹H NMR (DMSO) δ (ppm): 2.45 (m, 4H), 2.50 (m, 4H), 7.375 (m, 1H), 7.50 (m, 2H), 7.75 (m, 6H), 8.10 (s, 1H), 8.25 (s, 1H), 8.50 (bs, 1H) MS: MH⁺ 393 HPLC: 98.2%

### EXAMPLE 3

### 4-(4-Ethylphenyl)-N-(2-morpholin-4-ylethyl)thiophene-2-carboxamide

The product (15.8 mg) is obtained according to the process of Example 1, using 4-ethylphenylboronic acid as cosubstrate.

Yield: 15% ¹H NMR (CDCl₃) δ (ppm): 1.25 (t, 3H), 2.50 (m, 4H), 2.60 (m, 2H), 2.70 (q, 2H), 3.55 (m, 2H), 3.75 (m, 4H), 6.62 (bs, 1H), 7.25 (d, 2H), 7.50 (m, 3H), 7.75 (s, 1H) MS: MH⁺ 345 HPLC: 99.4%

### EXAMPLE 4

### 4-[4-(Methylthio)phenyl]-N-(2-morpholin-4-ylethyl)thiophene-2-carboxamide

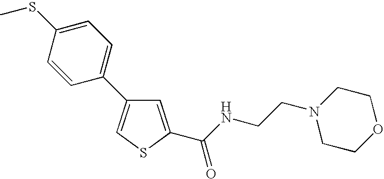

The product (64.4 mg) is obtained according to the process of Example 1, using 4-(methylthio)phenylboronic acid as cosubstrate.

Yield: 56% ¹H NMR (CDCl₃) δ (ppm): 2.52 (m, 7H), 2.60 (m, 2H), 3.55 (m, 2H), 3.75 (m, 4H), 6.625 (bs, 1H), 7.30 (d, 2H), 7.50 (m, 3H), 7.75 (s, 1H) MS: MH⁺ 363 HPLC: 94.9%

### EXAMPLE 5

### 4-[4-(Trifluorometboxy)phenyl]-N-(2-morpholin-4-ylethyl)thiophene-2-carboxamide

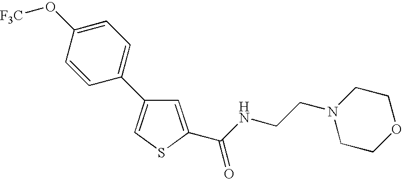

The product (166.2 mg) is obtained according to the process of Example 1, using 4-(trifluoromethoxy)phenylboronic acid as cosubstrate.

Yield: 44% ¹H NMR (CDCl₃) δ (ppm): 2.50 (m, 4H), 2.60 (m, 2H), 3.55 (m, 2H), 3.75 (m, 4H), 6.62 (bs, 1H), 7.25 (d, 2H), 7.55 (s, 1H), 7.60 (d, 2H), 7.75 (s, 1H) MS: MH⁺ 401 HPLC: 98.4%

### EXAMPLE 6

### 4-[4-(tert-Butyl)phenyl]-N-(2-morpholin-4-ylethyl)thiophene-2-carboxamide

### Stage 1: 4-[4-(tert-Butyl)phenyl]thiophene-2-carbaldehyde

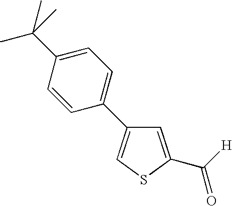

The product (199.8 mg) is obtained according to the process of Stage 1 of Preparation 9, using 4-bromothiophene-2-carbaldehyde as substrate and 4-(tert-butyl)phenylboronic acid as cosubstrate.

Yield: 78%

### Stage 2: 4-[4-(tert-Butyl)phenyl]thiophene-2-carboxylic acid

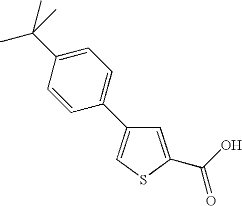

The product (39.3 mg) is obtained according to the process of Stage 2 of Preparation 5, using the compound obtained in the preceding Stage 1 as substrate.

Yield: 37%

### Stage 3: 4-[4-(tert-Butyl)phenyl]-N-(2-morpholin-4-ylethyl)thiophene-2-carboxamide

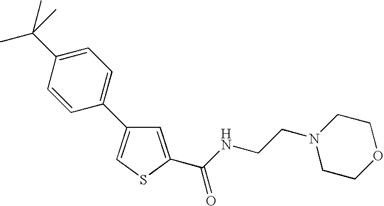

21.8 μl of 2-morpholin-4-ylethanamine (1.1 equivalents), 63.2 mg of O-(7-azabenzotriazol-1-yl)-N,N,N′,N′-tetramethyluronium hexafluorophosphate (HATU) and 57.9 μl of N-ethyl-N,N-diisopropylamine are added to a solution of 39.3 mg of the compound obtained in the preceding Stage 2 in 1.0 ml of anhydrous dimethylformamide. The reaction medium is stirred at ambient temperature for 17 hours and is then concentrated under reduced pressure. The residue obtained is dissolved in ethyl acetate (15 ml), washed with water (2×8 ml), dried over sodium sulphate, filtered and concentrated under reduced pressure. Chromatography of the residue on silica gel (dichloromethane/methanol: 98/2) makes it possible to isolate 9.0 mg of the expected product.

Yield: 16% ¹H NMR (CDCl₃) δ (ppm): 2.55 (m, 4H), 2.60 (m, 2H), 3.55 (m, 2H), 3.75 (m, 4H), 6.65 (bs, 1H), 7.45 (d, 2H), 7.52 (m, 3H), 7.75 (s, 1H) MS: MH⁺ 373 HPLC: 95.8%

### EXAMPLE 7

### 4-[4-(Trifluoromethyl)phenyl]-N-(2-morpholin-4-ylethyl)thiophene-2-carboxamide

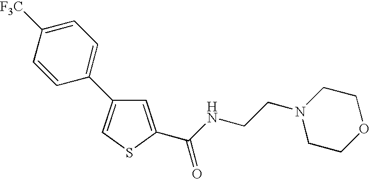

The product (16.3 mg) is obtained according to the process of Example 6, Stages 1 to 3, using 4-(trifluoromethyl)phenylboronic acid as cosubstrate in Stage 1 instead of 4-(tert-butyl)phenylboronic acid.

Yield: 26% ¹H NMR (CDCl₃) δ (ppm): 2.55 (m, 4H), 2.65 (m, 2H), 3.67 (m, 2H), 3.75 (m, 4H), 6.72 (bs, 1H), 7.65 (s, 1H), 7.675 (s, 4H), 7.80 (s, 1H) MS: MH⁺ 385 HPLC: 97.2%

### EXAMPLE 8

### 4-(4-Hydroxyphenyl)-N-(2-morpholin-4-ylethyl)thiophene-2-carboxamide

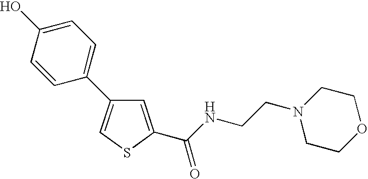

The product (2.73 g) is obtained according to the process of Example 1, using (4-hydroxyphenyl)boronic acid as cosubstrate instead of (4-isopropylphenyl)boronic acid.

Yield: 54% ¹H NMR (DMSO) δ (ppm): 2.42 (m, 4H), 2.50 (m, 2H), 3.40 (m, 2H), 3.60 (m, 4H), 6.80 (d, 2H), 7.50 (d, 2H), 7.85 (s, 1H), 8.10 (s, 1H), 8.42 (bs, 1H), 9.55 (s, 1H) MS: MH⁺ 333 HPLC: 97.8%

### EXAMPLE 9

### 4-[4-(Pyridin-4-yl)phenyl]-N-(2-morpholin-4-ylethyl)thiophene-2-carboxamide

### Stage 1: 4-(5-{[(2-Morpholin-4-ylethyl)amino]carbonyl}thien-3-yl)phenyl trifluoromethanesulphonate

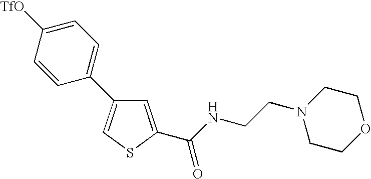

0.798 ml of pyridine (1.2 equivalents) and 1.45 ml of trifluoromethanesulphonic anhydride (1.1 equivalents) are added dropwise to a solution of 2.73 g of the compound obtained in Example 8 in 50 ml of anhydrous dichloromethane. The reaction medium is stirred at ambient temperature for 2 hours, diluted with 250 ml of dichloromethane, washed with water (3×100 ml), dried over sodium sulphate and concentrated under reduced pressure. Chromatography of the residue obtained on silica gel (dichloromethane/methanol: 98/2) makes it possible to isolate 2.62 g of the desired product.

Yield: 68% ¹H NMR (CDCl₃) δ (ppm): 2.47 (m, 4H), 2.55 (m, 2H), 3.50 (m, 2H), 3.70 (m, 4H), 6.50 (bs, 1H), 7.27 (d, 2H), 7.50 (s, 1H), 7.60 (d, 2H), 7.70 (s, 2H) MS: MH⁺ 465 HPLC: 98.1%

### Stage 2: 4-[4-(Pyridin-4-yl)phenyl]-N-(2-morpholin-4-ylethyl)thiophene-2-carboxamide

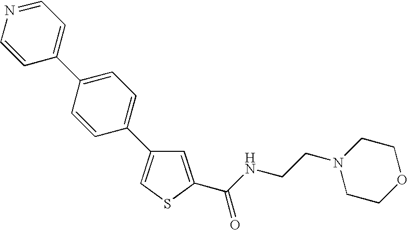

The product (23.7 mg) is obtained according to the process of Example 1, using the product obtained in the preceding Stage 1 as substrate and (4-pyridyl)boronic acid as cosubstrate instead of (4-isopropylphenyl)boronic acid.

Yield: 17% ¹H NMR (CDCl₃) δ (ppm): 2.55 (m, 4H), 2.65 (m, 2H), 3.60 (m, 2H), 3.75 (m, 4H), 6.65 (bs, 1H), 7.55 (d, 2H), 7.65 (s, 1H), 7.70 (s, 4H), 7.82 (s, 1H), 8.70 (d, 2H) MS: MH⁺ 394 HPLC: 94.5%

### EXAMPLE 10

### Methyl 4′-[5-(2-morpholin-4-ylethylcarbamoyl)thiophen-3-yl]biphenyl-3-carboxylate

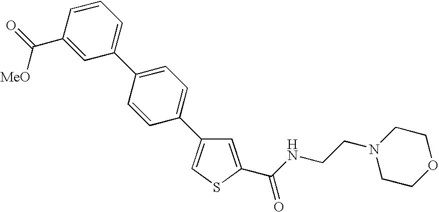

The product (200.2 mg) is obtained according to the process of Example 1, using the product of Stage 1 of Example 9 as substrate and [3-(methoxycarbonyl)phenyl]boronic acid as cosubstrate instead of (4-isopropylphenyl)boronic acid.

Yield: 68% ¹H NMR (CDCl₃) δ (ppm): 2.55 (m, 4H), 2.625 (m, 2H), 3.57 (m, 2H), 3.75 (m, 4H), 3.97 (s, 3H), 6.65 (bs, 1H), 7.55 (t, 1H), 7.625 (s, 1H), 7.70 (s, 4H), 7.82 (m, 2H), 8.05 (d, 1H), 8.30 (s, 1H) MS: MH⁺ 451 HPLC: 95.7%

### EXAMPLE 11

### 4-[4-(Pyridin-3yl)phenyl]-N-(2-morpholin-4-ylethyl)thiophene-2-carboxamide

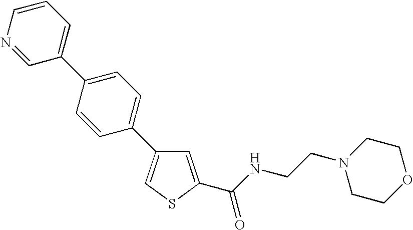

The product (95.2 mg) is obtained according to the process of Example 1, using the product obtained in Stage 1 of Example 9 as substrate and (3-pyridyl)boronic acid as cosubstrate instead of (4-isopropylphenyl)boronic acid.

Yield: 45% ¹H NMR (CDCl₃) δ (ppm): 2.55 (m, 4H), 2.65 (m, 2H), 3.60 (m, 2H), 3.75 (m, 4H), 6.65 (bs, 1H), 7.40 (m, 1H), 7.65 (m, 3H), 7.70 (d, 2H), 7.82 (s, 1H), 7.92 (d, 1H), 8.62 (d, 1H), 8.90 (s, 1H) MS: MH⁺ 394 HPLC: 100%

### EXAMPLE 12

### 4-[4-(Morpholin-4-yl)phenyl]-N-(2-morpholin-4-ylethyl)thiophene-2-carboxamide

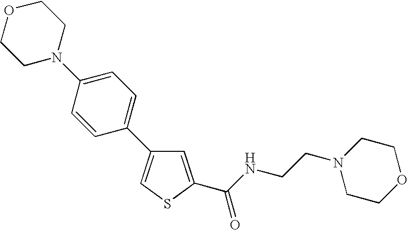

23 μl of morpholine (1.2 equivalents), 5.9 mg of tris(dibenzylideneacetone)dipalladium(0) (0.03 equivalent), 3.8 mg of 2-[di(t-butyl)phosphino]biphenyl (0.06 equivalent) and 64 mg of potassium phosphate are added under nitrogen to a solution of 100 mg of the compound obtained in Stage 1 of Example 9 in 2.0 ml of degassed DME. The reaction medium is stirred at 80° C. for 17 hours, diluted with ethyl acetate (20 ml), washed with water (2×10 ml), dried over sodium sulphate, filtered and concentrated under reduced pressure. Chromatography of the residue obtained on silica gel (dichloromethane/methanol: 98/2) makes it possible to isolate 25.2 mg of the expected product.

Yield: 29% ¹H NMR (CDCl₃) δ (ppm): 2.50 (m, 4H), 2.60 (m, 2H), 3.20 (m, 4H), 3.55 (m, 2H), 3.72 (m, 4H), 3.87 (m, 4H), 6.60 (bs, 1H), 6.97 (d, 2H), 7.45 (s, 1H), 7.50 (d, 2H), 7.75 (s, 1H) MS: MH⁺ 402 HPLC: 100%

### EXAMPLE 13

### 4-(4-Piperidinophenyl)-N-(2-morpholin-4-ylethyl)thiophene-2-carboxamide

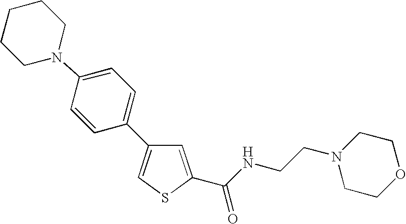

The product (40.3 mg) is obtained according to the process of Example 12, using piperidine as cosubstrate in place of morpholine.

Yield: 50% ¹H NMR (CDCl₃) δ (ppm): 1.60 (m, 2H), 1.725 (m, 4H), 2.50 (m, 4H), 2.60 (m, 2H), 3.20 (m, 4H), 3.52 (m, 2H), 3.75 (4H), 6.60 (bs, 1H), 7.00 (d, 2H), 7.42 (s, 1H), 7.50 (d, 2H), 7.72 (s, 1H) MS: MH⁺ 400 HPLC: 98.3%

### EXAMPLE 14

### 4-[4-(Pyrrolidin-1-yl)phenyl]-N-(2-morpholin-4-ylethyl)thiophene-2-carboxamide

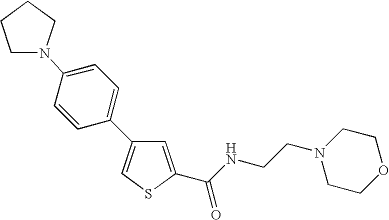

The product (45.7 mg) is obtained according to the process of Example 12, using pyrrolidine as cosubstrate in place of morpholine.

Yield: 36% ¹H NMR (CDCl₃) δ (ppm): 2.05 (m, 4H), 2.55 (m, 4H), 2.62 (m, 2H), 3.35 (m, 4H), 3.55 (m, 2H), 3.75 (m, 4H), 6.60 (m, 3H), 7.35 (s, 1H), 7.45 (d, 2H), 7.75 (s, 2H) MS: MH⁺ 386 HPLC: 99.2%

### EXAMPLE 15

### 4-[4-(1,4-Dioxa-8-azaspiro[4,5]dec-8-yl)phenyl]-N-(2-morpholin-4-ylethyl)thiophene-2-carboxamide

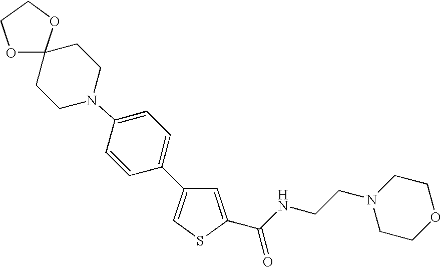

The product (82.1 mg) is obtained according to the process of Example 12, using 1,4-dioxa-8-azaspiro[4,5]decane as cosubstrate in place of morpholine.

Yield: 41% ¹H NMR (CDCl₃) δ (ppm): 1.87 (m, 4H), 2.52 (m, 4H), 2.62 (m, 2H), 3.40 (m, 4H), 3.55 (m, 2H), 3.75 (m, 4H), 4.00 (s, 4H), 6.62 (bs, 1H), 7.97 (d, 2H), 7.45 (s, 1H), 7.50 (d, 2H), 7.75 (s, 1H) MS: MH⁺ 458 HPLC: 100%

### EXAMPLE 16

### 4-[4-(1,2,3,6-Tetrahydropyridin-1-yl)phenyl]-N-(2-morpholin-4-ylethyl)thiophene-2arboxamide

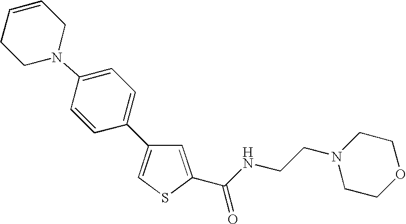

The product (33.4 mg) is obtained according to the process of Example 12, using 1,2,3,6-tetrahydropyridine as cosubstrate in place of morpholine.

Yield: 26% ¹H NMR (CDCl₃) δ (ppm): 2.25 (m, 2H), 2.45 (m, 4H), 2.55 (m, 2H), 3.37 (m, 2H), 3.50 (m, 2H), 3.67 (s, 6H), 5.75 (m, 1H), 5.85 (m, 1H), 6.65 (bs, 1H), 6.90 (d, 2H), 7.35 (s, 1H), 7.45 (d, 2H), 7.67 (s, 1H) MS: MH⁺ 398 HPLC: 94.0%

### EXAMPLE 17

### 4-[4-(3-(3R)-Hydroxypyrrolidin-1-yl)phenyl]-N-(2-morpholin-4-ylethyl)thiophene-2carboxamide

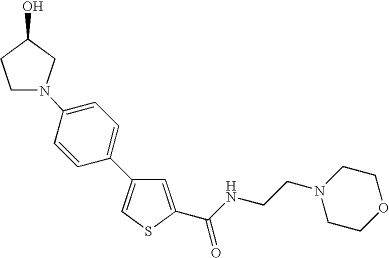

The product (66.5 mg) is obtained according to the process of Example 12, using (3R)-pyrrolidin-3-ol as cosubstrate in place of morpholine.

Yield: 38% ¹H NMR (CDCl₃) δ (ppm): 2.05 (m, 1H), 2.15 (m, 1H), 2.45 (m, 4H), 2.55 (m, 2H), 3.25 (m, 1H), 3.35 (m, 1H), 3.50 (m, 4H), 3.65 (m, 4H), 4.55 (bs, 1H), 6.55 (d, 3H), 7.30 (s, 1H), 7.40 (d, 2H), 7.65 (s, 1H) MS: MH⁺ 402 HPLC: 100%

### EXAMPLE 18

### 4-[4-(1H-Imidazol-1-yl)phenyl]-N-(2-morpholin-4-ylethyl)thiophene-2-carboxamide

### Stage 1: Methyl 4-bromothiophene-2-carboxylate

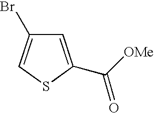

A few drops of concentrated sulphuric acid are added to a solution of 5.0 g of the compound obtained during Stage 1 of Preparation 1 in 25 ml of methanol. The reaction medium is stirred at 65° C. for 17 hours and is then concentrated under reduced pressure. The oily residue obtained is dissolved in ethyl acetate (200 ml), washed with water (3×100 ml), dried over sodium sulphate and then concentrated under reduced pressure in order to produce 5.24 g of the desired product.

Yield: 98% ¹H NMR (CDCl₃) δ (ppm): 3.90 (s, 3H), 7.45 (s, 1H), 7.70 (s, 1H)

### Stage 2: Methyl 4-(4-hydroxyphenyl)thiophene-2-carboxylate

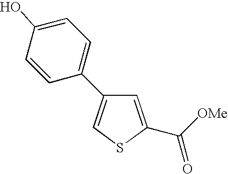

The product (1.43 g) is obtained according to the process of Example 1, using the product obtained in the preceding Stage 1 as substrate and (4-hydroxyphenyl)boronic acid as cosubstrate in place of (4-isopropylphenyl)boronic acid.

Yield: 37% ¹H NMR (CDCl₃) δ (ppm): 3.82 (s, 3H), 5.25 (s, 1H), 6.80 (d, 2H), 7.40 (d, 2H), 7.47 (s, 1H), 7.95 (s, 1H) HPLC: 98.8%

### Stage 3: Methyl 4-{4-[(trifluoromethylsulphonyl)oxy]phenyl}thiophene-2-carboxylate

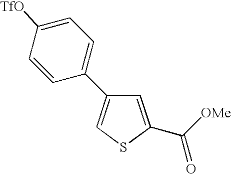

The product (1.74 g) is obtained according to the process of Stage 1 of Example 9, using the product obtained in the preceding Stage 2 as substrate.

Yield: 78% ¹H NMR (CDCl₃) δ (ppm): 4.925 (s, 3H), 7.35 (d, 2H), 7.675 (m, 3H), 8.05 (s, 1H) HPLC: 100%

### Stage 4: Methyl 4-[4-(pinacolboro)phenyl]thiophene-2-carboxylate

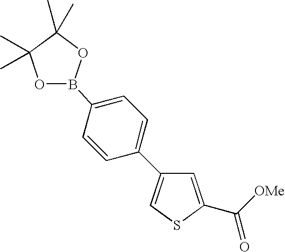

1.34 g of bis(pinacolato)diboron (1.2 equivalents), 96.3 mg of dichloro[1,1′-bis(diphenylphosphino)ferrocene]palladium(II) (0.03 equivalent), 146.1 mg of 1,1′-bis(diphenylphosphino)ferrocene (0.06 equivalent) and 1.29 g of potassium acetate (3 equivalents) are added under nitrogen to a solution of 1.61 g of the product obtained in the preceding Stage 3 in 35 ml of degassed 1,4-dioxane. The reaction medium is stirred at 80° C. for 17 hours, diluted with ethyl acetate (100 ml), washed with water (3×60 ml), dried over sodium sulphate and concentrated under reduced pressure. Chromatography of the residue on silica gel (dichloromethane: 100%) makes it possible to obtain 1.11 g of the desired product.

Yield: 73% ¹H NMR (CDCl₃) δ (ppm): 1.30 (s, 12H), 3.82 (s, 3H), 7.52 (d, 2H), 7.65 (s, 1H), 7.80 (d, 2H), 8.05 (s, 1H) HPLC:89%

### Stage 5: Methyl 4-[4-(dihydroxyboro)phenyl]thiophene-2-carboxylate

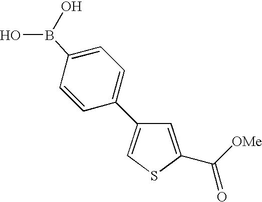

10 ml of water and 2.07 g of sodium periodate (3.0 equivalents) are added to a solution of 1.11 g of the compound obtained in the preceding Stage 4 in 10 ml of acetone. The reaction medium is stirred at 60° C. for 17 hours and is then concentrated under reduced pressure. The residue obtained is dissolved in ethyl acetate (50 ml), washed with a 1.0 mol/l hydrochloric acid solution and then with water (3×20 ml), dried over sodium sulphate, filtered and then concentrated under reduced pressure. Chromatography of the residue on silica gel (dichloromethane/methanol: 90/10) makes it possible to obtain 608 mg of the desired product.

Yield: 71% ¹H NMR (DMSO) δ (ppm): 3.85 (s, 3H), 7.75 (d, 2H), 7.85 (d, 2H), 8.22 (s, 1H), 8.30 (s, 1H) MS: MH⁻(+HCO₂H) 307

### Stage 6: Methyl 4-[4-(1H-imidazol-1-yl)phenyl]thiophene-2-carboxylate

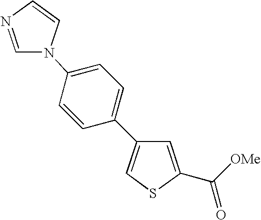

24 mg of imidazole (0.5 equivalent), 103.5 mg of copper (II) acetate (0.75 equivalent), 61 μl of pyridine (1 equivalent) and 20 mg of 4 Å molecular sieve are added to a solution of 200 mg of the compound obtained in Stage 5 in 2.5 ml of anhydrous dichloromethane. The reaction medium is stirred at ambient temperature for 17 hours, filtered, diluted with dichloromethane (20 ml), washed with water (2×10 ml), dried over sodium sulphate, filtered and then concentrated under reduced pressure. Chromatography of the residue on silica gel (dichloromethane/methanol: 92/10) makes it possible to obtain 40 mg of the desired product.

Yield: 37% ¹H NMR (DMSO) δ (ppm): 3.87 (s, 3H), 7.35 (bs, 1H), 7.77 (d, 2H), 8.00 (d, 2H), 8.30 (s, 1H), 8.40 (s, 1H), 8.72 (bs, 1H) HPLC: 97.9%

### Stage 7: 4-[4-(1H-Imidazol-1-yl)phenyl]thiophene-2-carboxylic acid

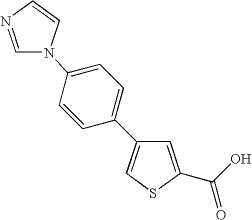

0.5 ml of water and 16.8 mg of lithium hydroxide (5 equivalents) are added to a solution of 40 mg of the compound obtained in Stage 6 in 1.0 ml of methanol. The reaction medium is stirred at 45° C. for 2 hours and is then concentrated under reduced pressure. The white solid obtained is taken up in 5.0 ml of a 1.0 mol/l hydrochloric acid solution. The precipitate formed is then filtered on a sintered glass funnel, washed with water (2×3.0 ml) and then dried in an oven overnight, thus making it possible to obtain 31 mg of the desired product.

Yield: 81% MS: MH⁺ 271 HPLC: 97.4%

### Stage 8: 4-[4-(1H-Imidazol-1-yl)phenyl]-N-(2-morpholin-4-ylethyl)thiophene-2-carboxamide

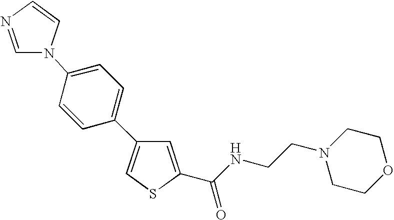

The product (22.1 mg) is obtained according to the process of Stage 3 of Example 6, using the product obtained in preceding Stage 7 as substrate.

Yield: 50% ¹H NMR (CDCl₃) δ (ppm): 2.50 (m, 4H), 2.55 (m, 2H), 3.50 (m, 2H), 3.70 (m, 2H), 6.57 (bs, 1H), 7.20 (m, 1H), 7.22 (s, 1H), 7.40 (d, 2H), 7.52 (s, 1H), 7.62 (d, 2H), 7.75 (s, 1H), 7.85 (s, 1H) MS: MH⁺ 383 HPLC: 97.9%

### EXAMPLE 19

### N-(2-Morpholin-4-ylethyl)-4-[4-(1H-pyrrol-1-yl)phenyl]thiophene-2-carboxamide

### Stage 1: Methyl 4-(4-nitrophenyl)thiophene-2-carboxylate

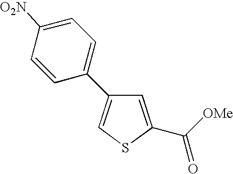

The product (1.94 g) is obtained according to the process of Example 1 using methyl 4-bromothiophene-2-carboxylate as substrate and (4-nitrophenyl)boronic acid as cosubstrate.

Yield: 78% ¹H NMR (CDCl₃) δ (ppm): 3.92 (s, 3H), 7.75 (d, 2H), 7.82 (s, 1H), 8.12 (s, 1H), 8.30 (d, 2H)

### Stage 2: Methyl 4-(4-aminophenyl)thiophene-2-carboxylate

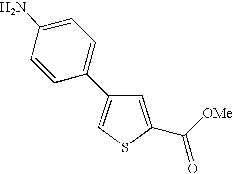

A solution of 1.94 g of the compound obtained in the preceding Stage 1 in 20 ml of methanol comprising 194 mg of 10% palladium-on-charcoal is stirred in an autoclave for 6 hours at 50° C. under 10 bar of hydrogen. The reaction medium is subsequently filtered through celite and concentrated under reduced pressure, making it possible to obtain 1.51 g of the desired product.

Yield: 88% ¹H NMR (DMSO) δ (ppm): 3.82 (s, 3H), 5.22 (s, 2H), 6.60 (d, 2H), 7.42 (d, 2H), 7.90 (s, 1H), 8.05 (s, 1H) MS: MH⁺ 234

### Stage 3: Methyl 4-[4-(1H-pyrrol-1-yl)phenyl]thiophene-2-carboxylate

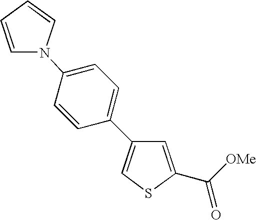

87 μl of 2,5-dimethoxytetrahydrofuran (1.05 equivalents) are added to a solution of 150 mg of the compound obtained in the preceding Stage 2 in 1.0 ml of acetic acid. The reaction medium is stirred at 110° C. for 1 hour, diluted with ethyl acetate (25 ml), washed with a 1.0 mol/l sodium hydroxide solution (10 ml) and then with water (2×10 ml), dried over sodium sulphate, filtered and finally concentrated under reduced pressure. Chromatography of the residue on silica gel (dichloromethane: 100%) makes it possible to obtain 96.3 mg of the desired product.

Yield: 53% ¹H NMR (CDCl₃) δ (ppm): 3.87 (s, 3H), 6.30 (s, 2H), 7.05 (s, 2H), 7.37 (d, 2H), 7.60 (m, 3H), 8.02 (s, 1H) HPLC: 95.9%

### Stage 4: 4-[4-(1H-Pyrrol-1-yl)phenyl]thiophene-2-carboxylic acid

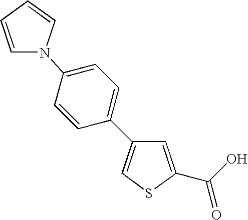

The product (69.5 mg) is obtained according to the process of Stage 7 of Example 18, using the product obtained in the preceding Stage 3 as substrate.

Yield: 81% ¹H NMR (DMSO) δ (ppm): 6.27 (s, 2H), 7.40 (s, 2H), 7.62 (d, 2H), 7.82 (d, 2H), 8.15 (s, 1H), 8.20 (s, 1H), 13.10 (bs, 1H) MS: MH⁻ 268 HPLC: 93.5%

### Stage 5: N-(2-Morpholin-4-ylethyl)-4-[4-(1H-pyrrol-1-yl)phenyl]thiophene-2-carboxamide

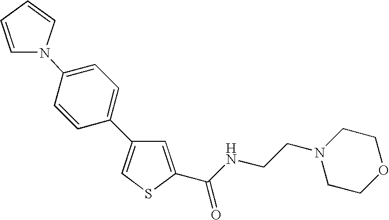

The product (99.1 mg) is obtained according to the process of Stage 3 of Example 6, using the product obtained in the preceding Stage 4 as substrate.

Yield: 57% ¹H NMR (CDCl₃) δ (ppm): 2.52 (m, 4H), 2.60 (m, 2H), 3.57 (m, 2H), 3.72 (m, 4H), 6.35 (s, 2H), 6.60 (bs, 1H), 7.12 (s, 2H), 7.45 (d, 2H), 7.55 (s, 1H), 7.625 (d, 2H), 7.77 (s, 1H) MS: MH⁺ 382 HPLC: 98.9%

### EXAMPLE 20

### 4-[4-(Isoxazol-5-yl)phenyl]-N-(2-morpholin-4-ylethyl)thiophene-2-carboxamide

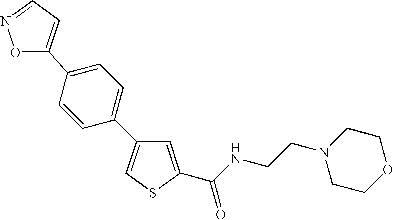

83.3 mg of 5-(4-bromophenyl)isoxazole (1.0 equivalent), 47.3 mg of lithium chloride (3.0 equivalents), 8.2 mg of copper(II) bromide (0.1 equivalent) and 12.9 mg of tetrakis(triphenylphosphine)palladium(0) (0.03 equivalent) are added under nitrogen to a solution of 150 mg of the compound obtained during Preparation 2 in 2.0 ml of degassed dioxane. The reaction medium is stirred at 80° C. for 4 hours, diluted with ethyl acetate (20 ml), washed with water (10 ml), dried over sodium sulphate, filtered and then concentrated under reduced pressure. Chromatography of the residue on silica gel (dichloromethane/methanol: 98/2) makes it possible to obtain 64.2 mg of the desired product.

Yield: 45% ¹H NMR (DMSO) δ (ppm): 2.45 (m, 4H), 2.52 (m, 2H), 3.40 (m, 2H), 3.60 (m, 4H), 7.75 (s, 1H), 7.82 (s, 4H), 8.17 (s, 1H), 8.25 (s, 1H), 8.47 (s, 1H), 8.55 (bs, 1H) MS: MH⁺ 384 HPLC: 100.0%

### EXAMPLE 21

### 4-[4-(Cyclohexyl)phenyl]-N-(2-morpholin-4-ylethyl)thiophene-2-carboxamide

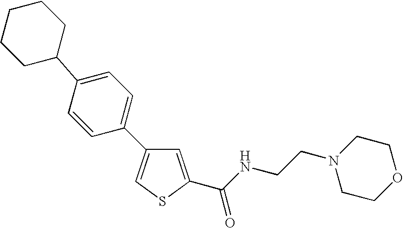

The product (14.8 mg) is obtained according to the process of Example 20, using 1-bromo-4-cyclohexylbenzene as cosubstrate.

Yield: 9% ¹H NMR (CDCl₃) δ (ppm): 1.30 (m, 1H), 1.45 (m, 4H), 1.75 (d, 1H), 1.85 (m, 4H), 2.55 (m, 5H), 2.65 (m, 2H), 3.55 (m, 2H), 3.75 (m, 4H), 6.75 (bs, 1H), 7.25 (d, 2H), 7.50 (m, 3H), 7.77 (s, 1H) MS: MH⁺ 399 HPLC: 97.0%

### EXAMPLE 22

### 4-[4-(1-Methyl-1H-pyrazol-3-yl)phenyl]-N-(2-morpholin-4-ylethyl)thiophene-2-carboxamide

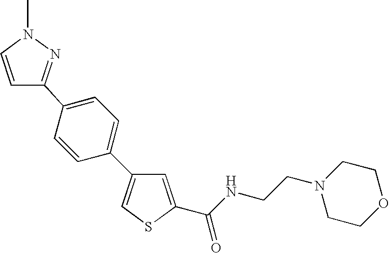

The product (36 mg) is obtained according to the process of Example 20, using 3-(4-bromophenyl)-1-methyl-1H-pyrazole as cosubstrate.

Yield: 23% ¹H NMR (DMSO) δ (ppm): 2.45 (m, 4H), 2.50 (m, 2H), 3.40 (m, 2H), 3.60 (m, 4H), 3.90 (m, 3H), 6.45 (m, 1H), 7.47 (s, 1H), 7.62 (d, 2H), 7.82 (d, 2H), 8.17 (s, 1H), 8.25 (s, 1H), 8.50 (bs, 1H) MS: MH⁺ 398 HPLC: 97.6%

### EXAMPLE 23

### 4-[4(6-Oxo-1,4,5,6-tetrahydropyridazin-3-yl)phenyl]-N-(2-morpholin-4-ylethyl)thiophene-2-carboxamide

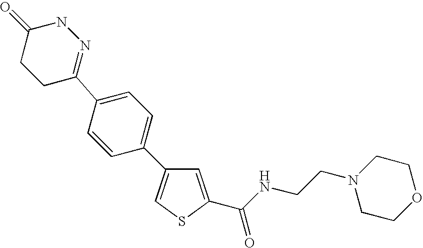

The product (8.1 mg) is obtained according to the process of Example 20, using 6-(4-bromophenyl)-4,5-dihydropyridazin-3(2H)-one as cosubstrate.

Yield: 5% ¹H NMR (DMSO) δ (ppm): 2.45 (m, 4H), 2.50 (m, 4H), 3.00 (t, 2H), 3.40 (m, 2H), 3.60 (m, 4H), 7.75 (d, 2H), 7.85 (d, 2H), 8.15 (s, 1H), 8.25 (s, 1H), 8.50 (bs, 1H), 10.95 (s, 1H) MS: MH⁺ 413 HPLC: 98.0%

### EXAMPLE 24

### Methyl trans-4-{[4-(4-phenylcyclohex-1-enyl)thiophene-2-carboxamido]methyl}cyclohexanecarboxylate

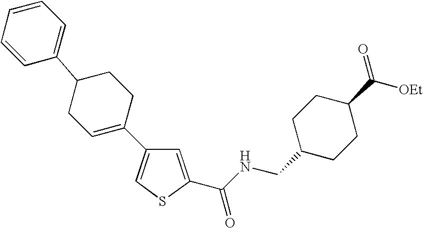

80 mg of the compound from Preparation 4 (1.2 equivalents), 10 mg of tris(dibenzylideneacetone)dipalladium(0) (0.05 equivalent) and 6.6 mg of triphenylarsine (0.1 equivalent) are added under nitrogen to a solution of 100 mg of the product from Preparation 3 in 2.0 ml of degassed dimethylformamide. The reaction medium is stirred at 45° C. for 2 hours, diluted with ethyl acetate (20 ml), washed with water (3×25 ml), dried over sodium sulphate, filtered and then concentrated under reduced pressure. Chromatography of the residue on silica gel (dichloromethane/methanol: 98/2) makes it possible to isolate 61.2 mg of the expected product.

Yield: 62% ¹H NMR (CDCl₃) δ (ppm): 1.05 (q, 2H), 1.25 (t, 3H), 1.45 (q, 2H), 1.60 (m, 1H), 1.90 (d, 3H), 2.05 (d, 2H), 2.12 (m, 1H), 2.25 (m, 1H), 2.35 (m, 1H), 2.52 (m, 3H), 2.85 (m, 1H), 3.30 (t, 2H), 4.10 (q, 2H), 6.00 (bs, 1H), 6.25 (bs, 1H), 7.25 (m, 4H), 7.32 (m, 2H), 7.65 (s, 1H) HPLC: 100.0%

### EXAMPLE 25

### trans-4-{[4-(4-Phenylcyclohex-1-enyl)thiophene-2-carboxamido]methyl}cyclohexanecarboxylic acid

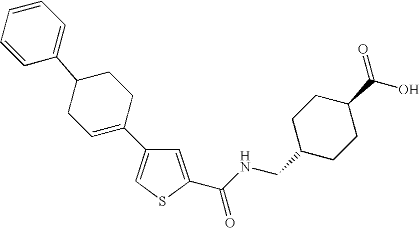

The product (48.2 mg) is obtained according to the process of Stage 7 of Example 18, using the product obtained during Example 24 as substrate.

Yield: 88% ¹H NMR (CDCl₃) δ (ppm): 1.05 (q, 2H), 1.45 (q, 2H), 1.62 (m, 1H), 1.90 (m, 3H), 2.10 (m, 3H), 2.35 (m, 2H), 2.55 (m, 3H), 2.90 (m, 1H), 3.30 (m, 2H), 6.05 (bs, 1H), 6.25 (bs, 1H), 7.25 (m, 4H), 7.35 (m, 2H), 7.65 (s, 1H) HPLC: 99.4%

### EXAMPLE 26

### Ethyl 3-(6-{[4-(4-trifluoromethoxyphenyl)thien-2-yl]carboxamido}-pyridin-3-yl)propanoate

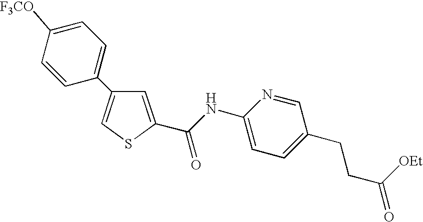

0.1 g (1.1 equivalents) of the compound from Preparation 6 and then 0.15 ml (2.1 equivalents) of triethylamine are added at 0° C. to a solution of 0.173 g of the compound from Preparation 5 in 5 ml of dichloromethane. After stirring for 17 hours at ambient temperature, the reaction medium is hydrolysed, washed successively with a 1.0M aqueous hydrochloric acid solution, a saturated NaHCO₃ solution and a saturated NaCl solution, then dried over sodium sulphate, filtered and evaporated under reduced pressure. Crystallization from diisopropyl ether makes it possible to isolate 0.093 g of the expected product in the form of a white solid.

Yield: 36% ¹H NMR (CDCl₃) δ (ppm): 8.6 (bs, 1H), 8.5 (s, 1H), 8.4 (m, 1H), 7.8 (m,2H), 7.7 (m, 2H), 7.6 (m, 2H), 7.3 (m, 2H), 4.1 (q, 2H), 3.0 (t, 2H), 2.6 (t, 2H), 1.2 (q, 3H) MS: MH⁺ 465 HPLC: 100%

### EXAMPLE 27

### 3-6-{[4-(4-Trifluoromethoxyphenyl)thien-2-yl]carboxamido}-pyridin-3-yl)propanoic acid

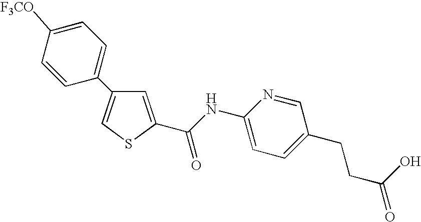

0.038 g (6 equivalents) of lithium hydroxide is added to a solution of 0.093 g of the compound of Example 26 in 6.0 ml of an ethanol/water (1/1: v/v) mixture. The reaction medium is stirred at ambient temperature for 17 hours and then concentrated under reduced pressure to remove the ethanol. The aqueous phase is acidified with a 1.0M aqueous hydrochloric acid solution and then extracted with ethyl acetate. The organic phase is dried over sodium sulphate, filtered and then concentrated under reduced pressure, making it possible to obtain 0.0087 g of the expected product.

Yield: 17% ¹H NMR (DMSO) δ (ppm): 12.5 (s, 1H), 8.8 (s, 1H), 8.3 (s, 1H), 8.1 (d, 1H), 7.9 (m, 1H), 7.7 (m, 2H), 7.6 (m, 2H), 3.0 (t, 2H), 2.6 (t, 2H) MS: MH⁺ 437, MH⁻ 435 HPLC: 97.2%

### EXAMPLE 28

### Ethyl 3-(4-{[4-(4-trifluoromethoxyphenyl)thien-2-yl]carboxamido}-phenyl)propenoate

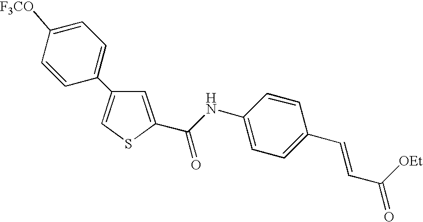

The product (0.146 g) is obtained according to the process of Example 26, using the product obtained in Preparation 7 as cosubstrate.

Yield: 48% ¹H NMR (CDCl₃) δ (ppm): 7.9 (s, 1H), 7.7 (bs, 1H), 7.6 (m, 6H), 7.5 (d, 2H), 7.25 (m, 2H), 6.4 (d, 1H), 4.2 (q, 2H), 1.25 (t, 3H) MS: MH⁻ 460 HPLC: 100%

### EXAMPLE 29

### 3-(4-{[4-(4-Trifluoromethoxyphenyl)thien-2-yl]carboxamido}-phenyl)propenoic acid

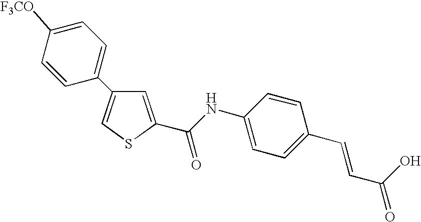

The product (0.094 g) is obtained according to the process of Example 27, using the compound obtained in Example 28 as substrate.

Yield: 69% ¹H NMR (DMSO) δ (ppm): 10.45 (s, 1H), 8.5 (s, 1H), 8.3 (s, 1H), 7.85 (dd, 2H), 7.8 (dd, 2H), 7.7 (dd, 2H), 7.6 (d, 1H), 7.5 (m, 2H), 6.4 (d, 1H) MS: MH⁻ 432 HPLC: 96.2%

### EXAMPLE 30

### Diethyl 4-({[4-(trifluoromethoxyphenyl)thien-2-yl]carboxamido}-methyl)phthalate

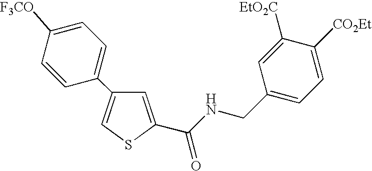

The product (0.106 g) is obtained according to the process of Example 26, using the product obtained in Preparation 8 as cosubstrate.

Yield: 22% ¹H NMR (CDCl₃) δ (ppm): 7.7 (s, 1H), 7.6 (m, 1H), 7.6 (m, 1H), 7.5 (m, 4H), 7.2 (m, 1H), 6.4 (bs, 1H), 4.8 (d, 2H), 4.3 (m, 4H), 1.4 (t, 6H) HPLC: 91%

### EXAMPLE 31

### 4-({[4-(Trifluoromethoxyphenyl)thien-2-yl]carboxamido}methyl)-phthalic diacid

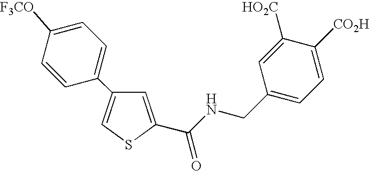

The product (0.0729 g) is obtained according to the process of Example 27, using the compound obtained in Example 30 as substrate.

Yield: 77.5% ¹H NMR (DMSO) δ (ppm): 9.2 (bs, 1H), 8.3 (s, 1H), 8.2 (s, 1H), 7.85 (m, 2H), 7.7 (m, 2H), 7.5 (dd, 1H), 7.4 (dd, 2H), 4.5 (d, 2H) MS: MH⁻ 464 HPLC: 96.2%

### EXAMPLE 32

### Ethyl 3-(4-{[4-(4-trifluoromethoxyphenyl)thien-2-yl]carboxamido}-phenyl)propanoate

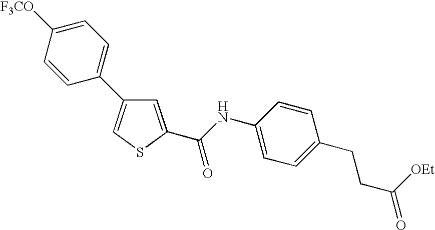

The product (0.483 g) is obtained according to the process of Example 26, using the product obtained in Preparation 9 as cosubstrate.

Yield: 80% ¹H NMR (CDCl₃) δ (ppm): 7.9 (s, 1H), 7.7 (m, 4H), 7.5 (m, 2H), 7.25 (m, 3H), 4.1 (q, 2H), 2.9 (t, 2H), 2.6 (t, 2H), 1.25 (t, 3H) MS: MH⁺ 464, MH⁻ 462 HPLC: 100%

### EXAMPLE 33

### 3-(4-{[4-(4-Trifluoromethoxyphenyl)thien-2-yl]carboxamido}-phenyl)propanoic acid

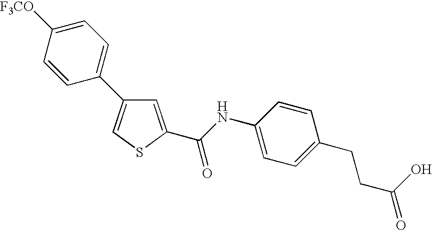

The product (0.279 g) is obtained according to the process of Example 27, using the compound obtained in Example 32 as substrate.

Yield: 61% ¹H NMR (DMSO) δ (ppm): 12.1 (s, 1H), 10.4 (s, 1H), 8.5 (s, 1H), 8.2 (s, 1H), 7.85 (dd, 2H), 7.6 (dd, 2H), 7.4 (dd, 2H), 7.2 (dd, 2H), 2.8 (t, 2H), 2.5 (m, 2H) MS: MH⁻ 434 HPLC: 100%

### EXAMPLE 34

### Methyl trans-4-({[4-(4-trifluoromethoxyphenyl)thien-2-yl]carbox-amido}methyl)cyclohexanecarboxylate

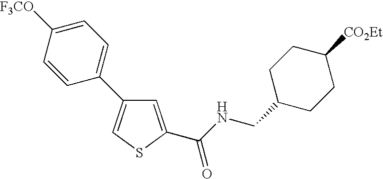

The product (0.120 g) is obtained according to the process of Example 26, using the product obtained in Preparation 10 as cosubstrate.

Yield: 41% ¹H NMR (CDCl₃) δ (ppm): 7.7 (s, 1H), 7.6 (m, 2H), 7.5 (s, 1H), 6.0 (bs, 1H), 4.1 (q, 2H), 3.4 (t, 2H), 2.25 (m, 1H), 2.0 (m, 2H), 1.9 (m, 2H), 1.6 (m, 1H), 1.4 (m, 2H), 1.25 (t, 3H), 1.10 (q, 2H) MS: MH⁺ 456, 500 (+HCOOH) HPLC: 98.6%

### EXAMPLE 35

### trans-4-({[4-(4-Trifluoromethoxyphenyl)thien-2-yl]-carboxamido}methyl)cyclohexanecarboxylic acid

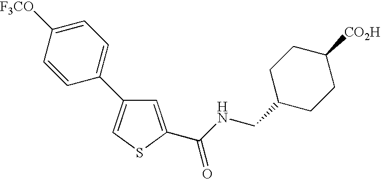

The product (0.112 g) is obtained according to the process of Example 27, using the compound obtained in Example 34 as substrate.

Yield: 100% ¹H NMR (DMSO) δ (ppm): 12.1 (s, 1H), 8.4 (bs, 1H), 8.2 (s, 1H), 8.1 (s, 1H), 7.85 (dd, 2H), 7.6 (dd, 2H), 3.1 (t, 2H), 2.1 (m, 1H), 1.8 (m, 2H), 1.75 (m, 2H), 1.5 (m, 1H), 1.2 (m, 2H), 1.0 (m, 2H) MS: MH⁻ 426 HPLC: 100%

### EXAMPLE 36

### Ethyl 2-[4-({[4-(4-trifluoromethoxyphenyl)thien-2-yl]carboxamido}-methyl)phenyl]acetate

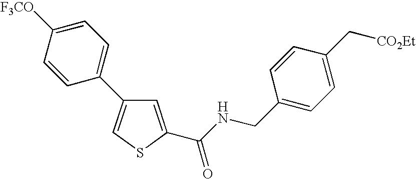

The product (0.036 g) is obtained according to the process of Example 26, using the product obtained in Preparation 11 as cosubstrate.

Yield: 27% ¹H NMR (CDCl₃) δ (ppm): 7.8 (s, 1H), 7.55 (m, 3H), 7.25 (m, 6H), 6.45 (bs, 1H), 4.60 (d, 2H), 4.1 (q, 2H), 3.6 (s, 2H), 1.25 (t, 3H) MS: MH⁺ 463.7, 508 (+HCOOH) HPLC: 94.9%

### EXAMPLE 37

### 2-[4-({[4-(4-Trifluoromethoxyphenyl)thien-2-yl]carboxamido}-methyl)phenyl]acetic acid

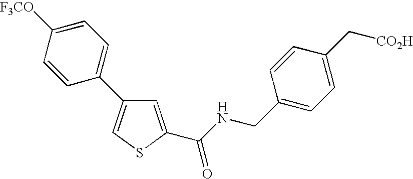

The product (0.0556 g) is obtained according to the process of Example 27, using the compound obtained in Example 36 as substrate.

Yield: 70% ¹H NMR (DMSO) δ (ppm): 12.1 (s, 1H), 9.1 (bs, 1H), 8.25 (s, 1H), 8.15 (s, 1H), 7.85 (m, 2H), 7.4 (m, 2H), 7.3 (m, 1H), 7.2 (m, 2H), 7.1 (m, 1H), 4.4 (d, 2H), 3.4 (s, 2H) MS: MH⁻ 480 HPLC: 100%

### EXAMPLE 38

### Ethyl 3-({[4-(4-trifluoromethoxyphenyl)thien-2-yl]carboxamido}-methyl)benzoate

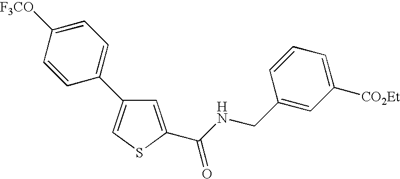

The product (0.070 g) is obtained according to the process of Example 26, using the product obtained in Preparation 12 as cosubstrate.

Yield: 20% ¹H NMR (CDCl₃) δ (ppm): 8.05 (bs, 1H), 8.0 (d, 1H), 7.7 (s, 1H), 7.5 (m, 4H), 7.4 (t, 1H), 7.2 (m, 2H), 6.3 (bs, 1H), 4.7 (d, 2H), 4.4 (q, 2H), 1.4 (t, 3H) MS: MH⁺ 450, 494 (+HCOOH) HPLC: 97.2%

### EXAMPLE 39

### 3-({[4-(4-Trifluoromethoxyphenyl)thien-2-yl]carboxamido}-methyl)benzoic acid

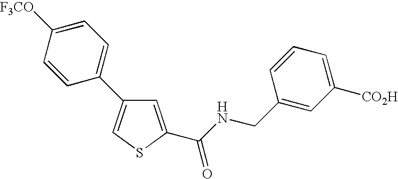

The product (0.0076 g) is obtained according to the process of Example 27, using the compound obtained in Example 38 as substrate.

Yield: 11% ¹H NMR (DMSO) δ (ppm): 12.1 (s, 1H), 9.1 (bs, 1H), 8.05 (bs, 1H), 8.0 (d, 1H), 7.7 (s, 1H), 7.5 (m, 4H), 7.4 (t, 1H), 7.2 (m, 2H), 4.7 (d, 2H) MS: MH⁺ 422, MH⁻ 420 HPLC: 100%

### EXAMPLE 40

### Ethyl 2-[3-({[4-(4-trifluoromethoxyphenyl)thien-2-yl]carboxamido}-methyl)phenyl]acetate

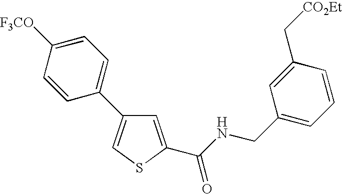

The product (0.038 g) is obtained according to the process of Example 26, using the product obtained in Preparation 13 as cosubstrate.

Yield: 27% ¹H NMR (CDCl₃) δ (ppm): 7.8 (s, 1H), 7.6 (m, 3H), 7.3 (m, 6H), 6.45 (bs, 1H), 4.65 (d, 2H), 4.1 (q, 2H), 3.7 (s, 2H), 1.2 (t, 3H) MS: MH⁺ 463.7, 508 (+HCOOH) HPLC: 94.9%

### EXAMPLE 41

### 2-[3-({[4-(4-Trifluoromethoxyphenyl)thien-2-yl]carboxamido}-methyl)phenyl]acetic acid

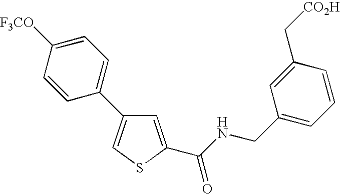

The product (0.023 g) is obtained according to the process of Example 27, using the compound obtained in Example 40 as substrate.

Yield: 65% ¹H NMR (DMSO) δ (ppm): 12.1 (s, 1H), 9.1 (bs, 1H), 8.30 (s, 1H), 8.2 (s, 1H), 7.7 (m, 2H), 7.5 (m, 2H), 7.3 (m, 1H), 7.2 (m, 2H), 7.1 (m, 1H), 4.7 (d, 2H), 3.6 (s, 2H) MS: MH⁺ 436, MH⁻ 435 HPLC: 98.4%

### EXAMPLE 42

### Ethyl 4-({[4-(4-trifluoromethoxyphenyl)thien-2-yl]carboxamido}-methyl)cyclohexanecarboxylate

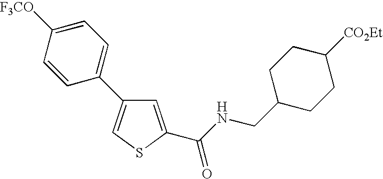

The product (0.116 g) is obtained according to the process of Example 26, using the product obtained in Preparation 14 as cosubstrate.

Yield: 23% ¹H NMR (CDCl₃) δ (ppm): 7.8 (s, 1H), 7.6 (m, 3H), 7.3 (m, 2H), 6.20 (bs, 1H), 4.10 (m, 2H), 3.3 (m, 2H), 2.1 (m, 1H), 2.05 (m, 2H), 1.9 (m, 2H), 1.7 (m, 1H), 1.45 (m, 2H), 1.3 (t, 3H), 1.0 (m, 2H) HPLC: 100%

### EXAMPLE 43

### 4-({[4-(4-Trifluoromethoxyphenyl)thien-2-yl]carboxamido}-methyl)cyclohexanecarboxylic acid

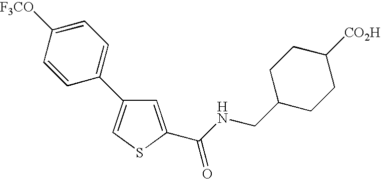

The product (0.082 g) is obtained according to the process of Example 27, using the compound obtained in Example 42 as substrate.

Yield: 78.5% ¹H NMR (DMSO) δ (ppm): 12.1 (s, 1H), 8.5 (bs, 1H), 8.30 (s, 1H), 8.2 (s, 1H), 7.7 (m, 2H), 7.5 (m, 2H), 3.1 (t, 2H), 2.2 (m, 1H), 1.9 (m, 2H), 1.8 (m, 2H), 1.6 (m, 1H), 1.3 (m, 2H), 1.0 (m, 2H) MS: MH⁻ 472 HPLC: 100%

### EXAMPLE 44

### Methyl 6-({[4-(4-trifluoromethoxyphenyl)thien-2-yl]carboxamido}-methyl)nicotinate

### Stage 1: 4-(4-Trifluoromethoxyphenyl)thiophene-2-carboxamide

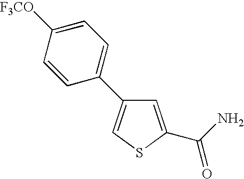

0.34 ml of a solution of ammonia in 5 ml of tetrahydrofuran is added, at −78° C., to a solution of 2.744 g of the compound from Preparation 5 in 25 ml of tetrahydrofuran. After stirring for 2 hours at ambient temperature, the reaction medium is concentrated under reduced pressure, hydrolysed with water and extracted with dichloromethane. The organic phase is washed with a saturated NaCl solution, dried over sodium sulphate, filtered and evaporated under reduced pressure. Crystallization makes it possible to isolate 1.569 g of the expected product in the form of a white solid.

Yield: 61.7% ¹H NMR (DMSO) δ (ppm): 8.2 (s, 1H), 8.1 (s, 1H), 8.0 (bs, 1H), 7.8 (m, 2H), 7.50 (m, 2H) HPLC: 96.92%

### Stage 2: Methyl 6-({[4-(4-trifluoromethoxyphenyl)thien-2-yl]carboxamido}methyl)-nicotinate

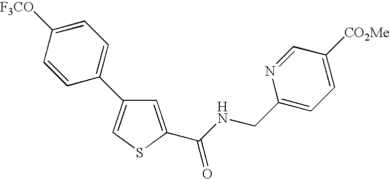

A solution of 0.0347 g of 60% sodium hydride in 2.0 ml of DMF at 0° C. is stirred at 40° C. for 2 hours. A solution of 0.249 g of the compound obtained in the preceding Stage 1 and of 0.2 g of the compound from Preparation 15 in 1 ml of DMF is stirred at 40° C. for 2 hours.

The solvent is evaporated under reduced pressure. The reaction medium is taken up in ethyl acetate, washed with water and dried over sodium sulphate to produce, after filtering and concentrating under reduced pressure, 0.139 g of a green gum. Purification on a semipreparative column makes it possible to obtain 0.012 g of a white powder corresponding to the expected product.

Yield: 3% MS: MH⁺ 437 HPLC: 100%

### EXAMPLE 45

### 6-({[4-(4-Trifluoromethoxyphenyl)thien-2-yl]carboxamido}-methyl)nicotinic acid

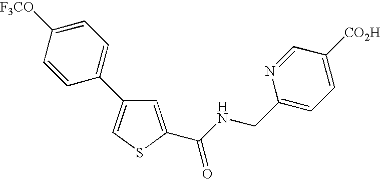

The product (0.006 g) is obtained according to the process of Example 27, using the compound obtained in Example 44 as substrate.

Yield: 79% ¹H NMR (DMSO) δ (ppm): 13.2 (s, 1H), 9.25 (bs, 1H), 9.0 (s, 1H), 8.3 (s, 1H), 8.25 (m, 1H), 8.2 (s, 1H), 7.8 (m, 2H), 7.6 (m, 3H), 4.6 (d, 2H) MS: MH⁺ 423, MH⁻ 421 HPLC: 100%

### EXAMPLE 46

### Ethyl 4-({[4-(4-trifluoromethoxyphenyl)thien-2-yl]carboxamido}-methyl)benzoate

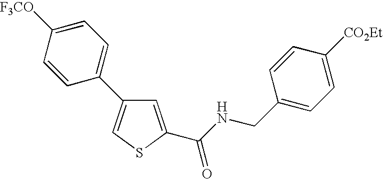

The product (0.051 g) is obtained according to the process of Example 1, using the compound obtained during Preparation 17 as substrate and [4-(trifluoromethoxy)phenyl]boronic acid as cosubstrate.

Yield: 42% MS: MH⁺ 450 HPLC: 94%

### EXAMPLE 47

### 4-({[4-(4-Trifluoromethoxyphenyl)thien-2-yl]carboxamido}-methyl)benzoic acid

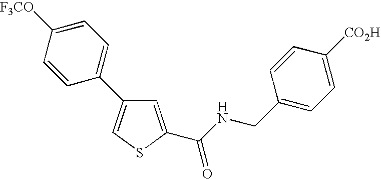

The product (0.008 g) is obtained according to the process of Example 27, using the compound obtained in Example 46 as substrate.

Yield: 17% ¹H NMR (DMSO) δ (ppm): 9.2 (bs, 1H), 8.3 (s, 1H), 8.2 (s, 1H), 7.85 (m, 2H), 7.7 (m, 2H), 7.4 (m, 4H), 4.5 (d, 2H) MS: MH⁻ 420 HPLC: 100%

### EXAMPLE 48

### Ethyl 4-({[4-(4-methylthiophenyl)thien-2-yl]carboxamido}methyl)-benzoate

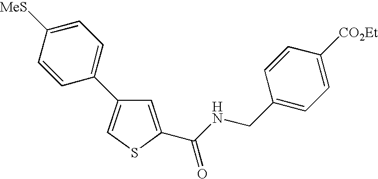

The product (0.1 g) is obtained according to the process of Example 46, using 4-(methylthio)phenylboronic acid as cosubstrate.

Yield: 42% ¹H NMR (CDCl₃) δ (ppm): 8.1 (m, 2H), 7.8 (s, 1H), 7.6 (s, 1H), 7.40 (m, 4H), 7.2 (m, 2H), 6.3 (bs, 1H), 4.7 (m, 2H), 4.4 (m, 2H), 2.5 (s, 3H), 1.4 (t, 3H) HPLC: 90.1%

### EXAMPLE 49

### 4-({[4-(4-Methylthiophenyl)thien-2-yl]carboxamido}methyl)benzoic acid

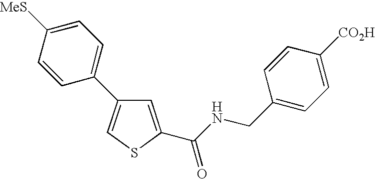

The product (0.0162 g) is obtained according to the process of Example 27, using the compound obtained in Example 48 as substrate. Yield: 17% ¹H NMR (DMSO) δ (ppm): 12.9 (s, 1H), 9.2 (bs, 1H), 8.2 (s, 1H), 8.1 (s, 1H), 7.9 (m, 2H), 7.7 (m, 2H), 7.4 (m, 2H), 7.2 (m, 2H), 4.4 (d, 2H), 3.3 (s, 3H) MS: MH⁺ 384, 428 (+HCOOH) HPLC: 96.3%

### EXAMPLE 50

### Ethyl 4-({[4-(4-(tert-butyl)phenyl)thien-2-yl]carboxamido}methyl)-benzoate

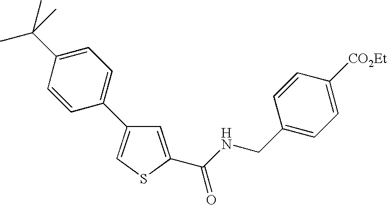

The product (0.16 g) is obtained according to the process of Example 46, using 4-(t-butyl)phenylboronic acid as cosubstrate.

Yield: 67% ¹H NMR (CDCl₃) δ (ppm): 8.0 (m, 2H), 7.8 (s, 1H), 7.6 (s, 1H), 7.40 (m, 2H), 7.2 (m, 4H), 6.3 (bs, 1H), 4.7 (m, 2H), 4.4 (m, 2H), 1.4 (t, 3H) MS: MH⁺ 422 HPLC: 100%

### EXAMPLE 51

### 4-({[4-(4-(tert-Butyl)phenyl)thien-2-yl]carboxamido}methyl)benzoic acid

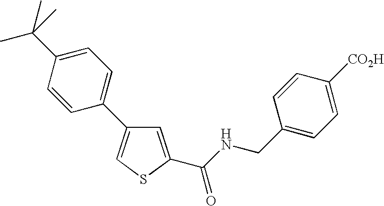

The product (0.152 g) is obtained according to the process of Example 27, using the compound obtained in Example 50 as substrate.

Yield: 85.4% ¹H NMR (DMSO) δ (ppm): 12.9 (s, 1H), 9.2 (bs, 1H), 8.2 (s, 1H), 8.1 (s, 1H), 7.85 (m, 2H), 7.6 (m, 2H), 7.4 (m, 4H), 4.5 (d, 2H), 1.3 (s, 9H) MS: MH⁺ 394, MH⁻ 392 HPLC: 100%

### EXAMPLE 52

### Ethyl (4-{[4-(4-(tert-butyl)phenyl)thien-2-yl]carboxamido}phenyl)-acetate

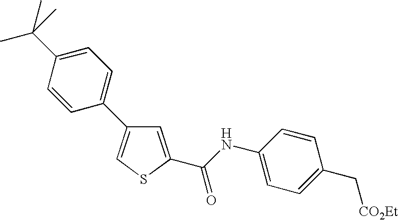

The product (0.141 g) is obtained according to the process of Example 46, using the compound obtained in Preparation 18 as substrate and 4-tert-butylphenylboronic acid.

Yield: 60% ¹H NMR (CDCl₃) δ (ppm): 7.9 (bs, 1H), 7.6 (s, 1H), 7.55 (m, 3H), 7.5 (m, 2H), 7.4 (m, 2H), 7.3 (m, 2H), 4.1 (q, 2H), 3.6 (s, 2H), 1.35 (s, 9H), 1.25 (t, 3H) MS: MH⁺ 422 HPLC: 100%

### EXAMPLE 53

### (4-{[4-(4-(tert-Butyl)phenyl)thien-2-yl]carboxamido}phenyl)acetic acid

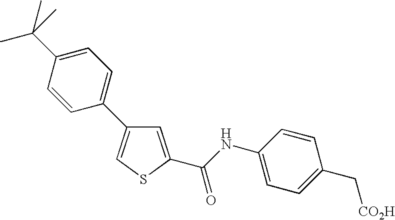

The product (0.113 g) is obtained according to the process of Example 27, using the compound obtained in Example 52 as substrate.

Yield: 85.4% ¹H NMR (DMSO) δ (ppm): 12.1 (s, 1H), 10.4 (s, 1H), 8.5 (s, 1H), 8.2 (s, 1 H), 7.7 (m, 4H), 7.6 (m, 2H), 7.4 (m, 2H), 3.5 (s, 2H), 1.4 (s, 9H) MS: MH⁺ 394, MH⁻ 392 HPLC: 100%

### EXAMPLE 54

### Ethyl (4-{[4-(4-trifluoromethoxyphenyl)thien-2-yl]carboxamido}-phenyl)acetate

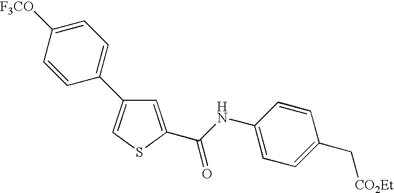

The product (0.0675 g) is obtained according to the process of Example 46, using the compound obtained in Preparation 18 as substrate and [4-(trifluoromethoxy)phenyl]-boronic acid as cosubstrate.

Yield: 40% ¹H NMR (CDCl₃) δ (ppm): 7.8 (s, 1H), 7.7 (s, 1H), 7.5 (m, 4H), 7.25 (m, 4H), 4.2 (q, 2H), 3.6 (s, 2H), 1.3 (t, 3H) MS: MH⁺ 449, 494 (+HCOOH) HPLC: 91%

### EXAMPLE 55

### (4-{[4-(4-Trifluoromethoxyphenyl)thien-2-yl]carboxamido}-phenyl)acetic acid

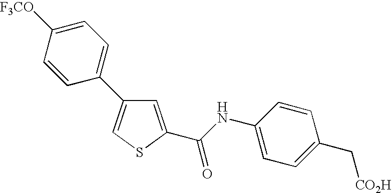

The product (0.080 g) is obtained according to the process of Example 27, using the compound obtained in Example 54 as substrate.

Yield: 54% ¹H NMR (DMSO) δ (ppm): 12.1 (s, 1H), 10.4 (s, 1H), 8.6 (s, 1H), 8.2 (s, 1H), 7.85 (m, 2H), 7.6 (m, 2H), 7.4 (m, 2H), 7.2 (m, 2H), 3.5 (s, 2H) MS: MH⁺ 422, MH⁻ 420 HPLC: 97.0%

### EXAMPLE 56

### Ethyl (4-{[4-(4-methylthiophenyl)thien-2-yl]carboxamido}phenyl)-acetate

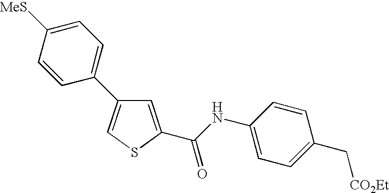

The product (0.119 g) is obtained according to the process of Example 46, using the compound obtained in Preparation 18 as substrate and 4-(methylthio)phenylboronic acid as cosubstrate.

Yield: 51% ¹H NMR (CDCl₃) δ (ppm): 7.8 (s, 1H), 7.6 (s, 1H), 7.5 (m, 3H), 7.45 (m, 2H), 7.3 (m, 4H), 4.2 (q, 2H), 3.7 (s, 2H), 2.5 (s, 3H), 1.3 (t, 3H) MS: MH⁻ 411 HPLC: 97.7%

### EXAMPLE 57

### (4-{[4-(4-Methylthiophenyl)thien-2-yl]carboxamido}phenyl)acetic acid

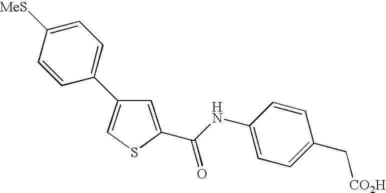

The product (0.083 g) is obtained according to the process of Example 27, using the compound obtained in Example 56 as substrate.

Yield: 75% ¹H NMR (DMSO) δ (ppm): 12.1 (s, 1H), 10.4 (s, 1H), 8.5 (s, 1H), 8.2 (s, 1H), 7.85 (m, 4H), 7.3 (m, 2H), 7.4 (m, 2H), 3.5 (s, 3H) MS: MH⁻ 382 HPLC: 100%

### EXAMPLE 58

### Ethyl 4-({[4-(4-methoxyphenyl)thien-2-yl]carboxamido}methyl)-benzoate

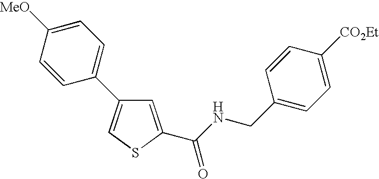

The product (0.300 g) is obtained according to the process of Example 26, using the product from Preparation 19 and ethyl 4-(aminomethyl)benzoate as substrates.

Yield: 52% ¹H NMR (DMSO) δ (ppm): 9.1 (bs, 1H), 8.2 (s, 1H), 7.9 (m, 3H), 7.6 (m, 2H), 7.4 (m, 2H), 7.0 (m, 2H), 4.5 (d, 2H), 4.3 (q, 2H), 3.8 (s, 3H), 1.3 (t, 3H) MS: MH⁺ 383 HPLC: 97.2%

### EXAMPLE 59

### 4-({[4-(4-Methoxyphenyl)thien-2-yl]carboxamido}methyl)benzoic acid

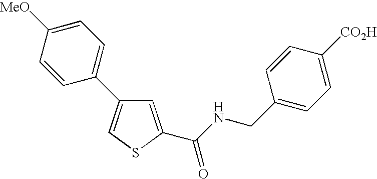

The product (0.060 g) is obtained according to the process of Example 27, using the compound obtained in Example 58 as substrate.

Yield: 31% ¹H NMR (DMSO) δ ppm: 12.9 (s, 1H), 9.1 (bs, 1H), 8.2 (s, 1H), 7.9 (s, 1H), 7.85 (m, 2H), 7.6 (m, 2H), 7.4 (m, 2H), 7.0 (m, 2H), 4.5 (d, 2H), 3.8 (s, 3H) MS: MH⁺ 368 HPLC: 100%

### EXAMPLE 60

### Ethyl (4-{[4-(4-methoxyphenyl)thien-2-yl]carboxamido}phenyl)-acetate

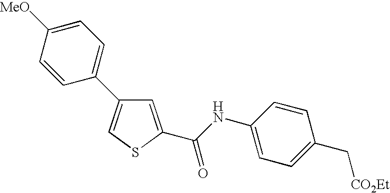

The product (0.16 g) is obtained according to the process of Example 26, using the product from Preparation 19 and that from Preparation 18, Stage 1, as substrates.

Yield: 93% ¹H NMR (DMSO) δ (ppm): 10.2 (bs, 1H), 8.4 (s, 1H), 7.9 (s, 1H), 7.6 (m, 4H), 7.3 (m, 2H), 7.0 (m, 2H), 4.1 (q, 2H), 3.8 (s, 3H), 3.6 (s, 2H), 1.3 (t, 3H) MS: MH⁺ 395 HPLC: 100%

### EXAMPLE 61

### (4-{[4-(4-Methoxyphenyl)thien-2-yl]carboxamido}phenyl)acetic acid

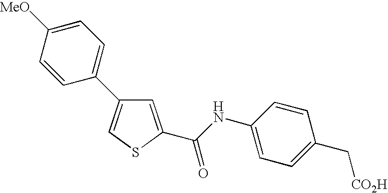

The product (0.0615 g) is obtained according to the process of Example 27, using the compound obtained in Example 60 as substrate.

Yield: 41% ¹H NMR (DMSO) δ (ppm): 12.2 (s, 1H), 10.2 (s, 1H), 8.5 (s, 1H), 8.0 (s, 1H), 7.75 (m, 4H), 7.3 (m, 2H), 7.0 (m, 2H), 3.8 (s, 3H), 3.5 (s, 2H) MS: MH⁺ 368, MH⁻ 366 HPLC: 100%

### EXAMPLE 62

### Ethyl 3-[4-({[4-(4-trifluoromethoxyphenyl)thien-2-yl]carboxamido}-methyl)phenyl]propanoate

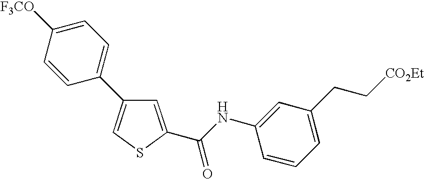

The product (0.084 g) is obtained according to the process of Example 26, using the product from Preparation 5 and the product from Preparation 20 as substrates.

Yield: 14% ¹H NMR (CDCl₃) δ ppm: 7.9 (s, 1H), 7.6 (m, 3H), 7.3 (m, 2H), 7.2 (m, 4H), 6.2 (bs, 1H), 4.6 (m, 2H), 4.2 (q, 2H), 3.6 (s, 2H), 1.3 (t, 3H) HPLC: 100%

### EXAMPLE 63

### 3-[4-({[4-(4-Trifluoromethoxyphenyl)thien-2-yl]carboxamido}-methyl)phenyl]propanoic acid

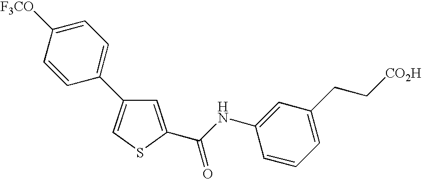

The product (0.0556 g) is obtained according to the process of Example 27, using the compound obtained in Example 62 as substrate.

Yield: 95% ¹H NMR (DMSO) δ (ppm): 12.1 (s, 1H), 9.1 (bs, 1H), 8.3 (s, 1H), 8.2 (s, 1H), 7.85 (m, 2H), 7.5 (m, 2H), 7.2 (m, 2H), 7.1 (m, 2H), 4.4 (d, 2H), 3.3 (m, 2H), 2.7 (m, 2H) MS: MH⁻ 494 HPLC: 100%

### EXAMPLE 64

### Ethyl 3-[3-({[4-(4-trifluoromethoxyphenyl)thien-2-yl]carboxamido}-methyl)phenyl]propanoate

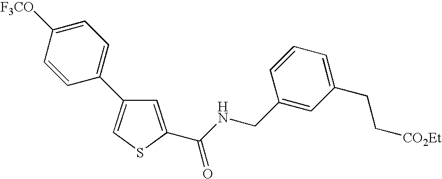

The product (0.243 g) is obtained according to the process of Example 26, using the product from Preparation 5 and the product from Preparation 21 as substrates.

Yield: 41.3% ¹H NMR (CDCl₃) δ ppm: 7.8 (s, 1H), 7.5 (m, 3H), 7.2 (m, 6H), 6.2 (bs, 1H), 4.7 (d, 2H), 4.1 (q, 2H), 3.0 (m, 2H), 2.6 (m, 2H), 1.2 (t, 3H) HPLC: 100%

### EXAMPLE 65

### 3-[3-({[4-(4-Trifluoromethoxyphenyl)thien-2-yl]carboxamido}-methyl)phenyl]propanoic acid

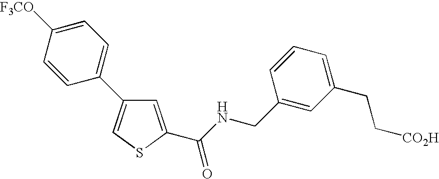

The product (0.1708 g) is obtained according to the process of Example 27, using the compound obtained in Example 64 as substrate.

Yield: 75% ¹H NMR (DMSO) δ (ppm): 12.1 (s, 1H), 9.1 (bs, 1H), 8.3 (s, 1H), 8.2 (s, 1H), 7.85 (m, 2H), 7.5 (m, 2H), 7.2 (m, 2H), 7.1 (m, 2H), 4.4 (d, 2H), 2.7 (m, 2H), 2.5 (m, 2H) MS: MH⁻ 448 HPLC: 100%

### EXAMPLE 66

### Ethyl 7-{[4-(4-trifluoromethoxyphenyl)thien-2-yl]carboxamido}-heptanoate

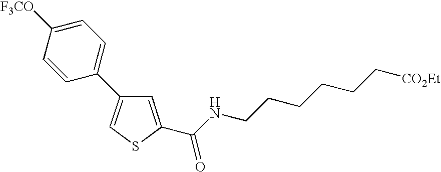

The product (0.363 g) is obtained according to the process of Example 26, using the product from Preparation 5 and the product from Preparation 22 as substrates.

Yield: 32% ¹H NMR (CDCl₃) δ (ppm): 7.8 (s, 1H), 7.7 (m, 2H), 7.6 (s, 1H), 7.3 (m, 2H), 6.3 (bs, 1H), 4.1 (m, 2H), 3.5 (q, 2H), 2.4 (t, 2H), 1.7 (m, 4H), 1.5 (m, 2H), 1.3 (t, 3H) HPLC: 100%

### EXAMPLE 67

### 7-{[4-(4-Trifluoromethoxyphenyl)thien-2-yl]carboxamido}-heptanoic acid

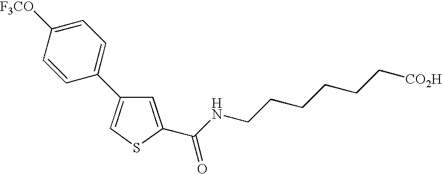

The product (0.284 g) is obtained according to the process of Example 27, using the compound obtained in Example 66 as substrate.

Yield: 84% ¹H NMR (DMSO) δ (ppm): 12.1 (s, 1H), 8.5 (bs, 1H), 8.3 (s, 1H), 8.2 (s, 1H), 7.85 (m, 2H), 7.5 (m, 2H), 3.4 (m, 2H), 2.4 (m, 2H), 1.5 (m, 4H), 1.3 (m, 2H) MS: MH⁺ 402, MH⁻ 400 HPLC: 100%

### EXAMPLE 68

### Ethyl 4-({[4-(4-hydroxyphenyl)thien-2-yl]carboxamido}methyl)-cyclohexanecarboxylate

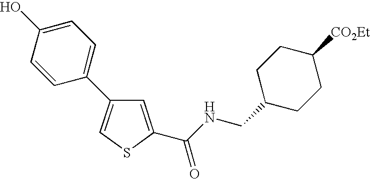

The product (0.974 g) is obtained according to the process of Preparation 5, Stage 1, using the compound obtained in Preparation 3, Stage 1, and (4-hydroxyphenyl)boronic acid as substrates.

Yield: 40% ¹H NMR (CDCl₃) δ (ppm): 7.7 (s, 1H), 7.4 (m, 3H), 6.8 (m, 2H), 6.2 (bs, 1H), 4.1 (q, 2H), 3.3 (m, 2H), 2.3 (m, 1H), 2.1 (m, 2H), 1.9 (m, 2H), 1.7 (m, 1H), 1.4 (m, 2H), 1.25 (t, 3H), 1.1 (m, 2H) MS: MH⁺ 388 HPLC: 100%

### EXAMPLE 69

### Ethyl 4-[({4-[4-(pyridin-4-yl)phenyl]thien-2-yl}carboxamido)-methyl]cyclohexanecarboxylate

### Stage 1: Ethyl 4-[({4-[4-(trifluoromethanesulphonyloxy)phenyl]thien-2-yl}carbox-amido)methyl]cyclohexanecarboxylate

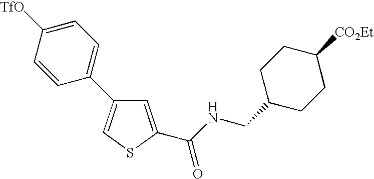

The product (1.136 g) is obtained according to the process of Example 18, Stage 3, using the product obtained in Example 68 as substrate.

Yield: 100% ¹H NMR (CDCl₃) δ (ppm): 7.8 (s, 1H), 7.7 (m, 2H), 7.6 (s, 1H), 7.3 (m, 2H), 6.3 (bs, 1H), 4.1 (q, 2H), 3.3 (m, 2H), 2.4 (m, 1H), 2.0 (m, 2H), 1.9 (m, 2H), 1.7 (m, 1.6 (m, 2H), 1.5 (t, 3H), 1.0 (m, 2H) MS: MH⁺ 520, 564 (+HCOOH) HPLC: 100%

### Stage 2: Ethyl 4-[({4-[4-(pyridin-4-yl)phenyl]thien-2-yl}carboxamido)methyl]cyclo-hexanecarboxylate

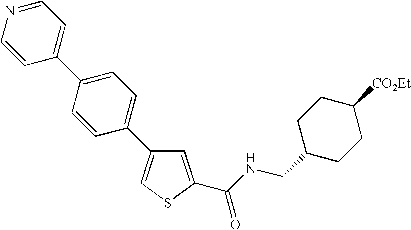

The product (0.133 g) is obtained according to the process of Preparation 5, Stage 1, using the compound obtained in the preceding Stage 1 as substrate and pyridine-4-boronic acid as cosubstrate.

Yield: 75% ¹H NMR (DMSO) δ (ppm): 12.1 (s, 1H), 9.1 (bs, 1H), 8.3 (s, 1H), 8.2 (s, 1H), 7.85 (m, 2H), 7.5 (m, 2H), 7.2 (m, 2H), 7.1 (m, 2H), 4.4 (d, 2H), 2.7 (m, 2H), 2.5 (m, 2H) MS: MH⁻ 448 HPLC: 100%

### EXAMPLE 70

### 4-[({4-[4-(Pyridinyl)phenyl]thien-2-yl}carboxamido)methyl]cyclo-hexanecarboxylic acid hydrochloride

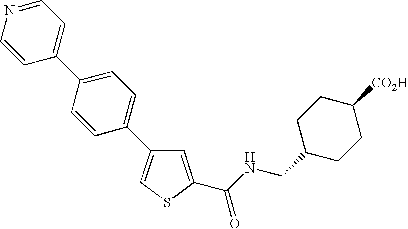

The product is obtained according to the process of Example 27, using the compound obtained in Example 69 as substrate. The solid is stirred in 2.0 ml of diethyl ether and 1.8 ml of a 1.0M HCl/Et₂O solution are added dropwise. The precipitate is filtered off, washed with water and dried at 60° C. under vacuum to produce the expected product (0.0507 g).

Yield: 15.2% ¹H NMR (DMSO) δ (ppm): 12.1 (s, 1H), 9.2 (m, 2H), 8.6 (s, 1H), 8.4 (m, 3H), 8.1 (m, 2H), 7.8 (m, 2H), 3.1 (d, 2H), 2.2 (m, 1H), 1.9 (m, 2H), 1.8 (m, 2H), 1.5 (m, 1H), 1.3 (m, 2H), 1.0 (m, 2H) MS: MH⁺ 421 HPLC: 97.6%

### EXAMPLE 71

### 4-(4-Bromophenyl)-N-(2-morpholin-4-ylethyl)thiophene-2-carboxamide

### Stage 1: Methyl 4-(4-bromophenyl)thiophene-2-carboxylate

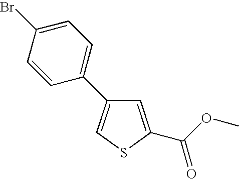

0.6 ml of concentrated hydrobromic acid is added to a solution of 103 mg of the product obtained during Stage 2 of Example 19 in 1.5 ml of water. The reaction medium is cooled to 0° C. and then a solution of 35.5 mg of sodium nitrite (1.1 equivalents) in 0.5 ml of water is added dropwise. After stirring at 0° C. for 1 hour, a solution of 68 mg of copper bromide in 0.5 ml of concentrated hydrobromic acid is added dropwise. The reaction medium is again stirred at 0° C. for 1 hour and is then diluted with ethyl acetate (30 ml), washed with water (3×15 ml), washed with a saturated sodium hydrogencarbonate solution (15 ml) and then again washed with water (15 ml). The organic phase is dried over sodium sulphate, filtered and then concentrated under reduced pressure. Chromatography of the residue on silica gel (cyclohexane/ethyl acetate: 95/5) makes it possible to isolate 52 mg of the desired product.

Yield: 40% ¹H NMR (CDCl₃) δ (ppm): 3.92 (s, 3H), 7.45 (d, 2H), 7.55 (d, 2H), 7.65 (s, 1H), 8.05 (s, 1H) HPLC: 91.4%

### Stage 2: 4-(4-Bromophenyl)thiophene-2-carboxylic acid

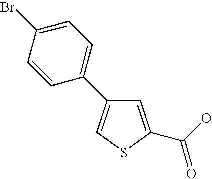

The product (11.32 g) is obtained according to the process of Stage 7 of Example 19, using the compound from the preceding Stage 1 as substrate.

Yield: 99% MS: MH⁻ 282 HPLC: 99.3%

### Stage 3: 4-(4-Bromophenyl)-N-(2-morpholin-4-ylethyl)thiophene-2-carboxamide

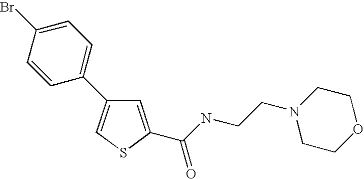

The product (11.58 g) is obtained according to the process of Stage 2 of Preparation 1, using the compound from the preceding Stage 2 as substrate.

Yield: 73% ¹H NMR (CDCl₃) δ (ppm): 2.45 (m, 4H), 2.55 (m, 2H), 3.50 (m, 2H), 3.67 (m, 4H), 6.60 (bs, 1H), 7.35 (d, 2H), 7.45 (m, 3H), 7.67 (s, 1H) MS: MH⁺ 396 HPLC: 97.3%

### EXAMPLE 72

### 4-[4-(4-Acetylphenyl)phenyl]-N-(2-morpholin-4-ylethyl)thiophene-2-carboxamide

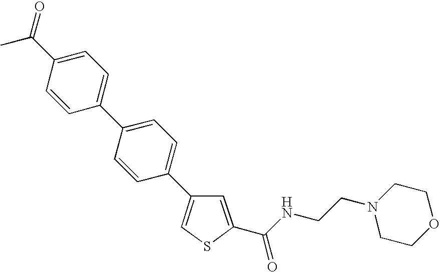

The product (86.8 mg) is obtained according to the process of Stage 1 of Preparation 5, using the compound of Example 71 as substrate and (4-acetylphenyl)boronic acid as cosubstrate.

Yield: 78% ¹H NMR (DMSO) δ (ppm): 2.45 (m, 4H), 2.50 (m, 2H), 2.62 (s, 3H), 3.40 (m, 2H), 3.60 (m, 4H), 7.85 (m, 6H), 8.05 (m, 2H), 8.17 (s, 1H), 8.27 (s, 1H), 8.50 (bs, 1H) MS: MH⁺ 435 HPLC: 98.1%

### EXAMPLE 73

### 4-[4-(4-Fluorophenyl)phenyl]-N-(2-morpholin-4-ylethyl)thiophene-2-carboxamide

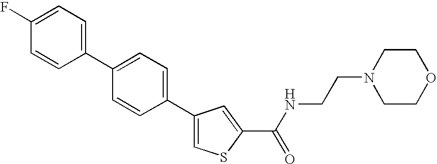

The product (84.5 mg) is obtained according to the process of Example 72, using (4-fluorophenyl)boronic acid as cosubstrate.

Yield: 81% ¹H NMR (DMSO) δ (ppm): 2.45 (m, 4H), 2.50 (m, 2H), 3.40 (m, 2H), 3.60 (m, 4H), 7.30 (m, 2H), 7.80 (m, 6H), 8.125 (s, 1H), 8.25 (s, 1H), 8.50 (bs, 1H) MS: MH⁺ 411 HPLC: 100%

### EXAMPLE 74

### 4-[4-(3-Hydroxyphenyl)phenyl]-N-(2-morpholin-4-ylethyl)thiophene-2-carboxamide

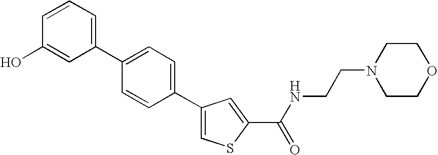

The product (61.5 mg) is obtained according to the process of Example 72, using (3-hydroxyphenyl)boronic acid as cosubstrate.

Yield: 59% ¹H NMR (DMSO) δ (ppm): 2.45 (m, 4H), 2.50 (m, 2H), 3.40 (m, 2H), 3.60 (m, 4H), 6.80 (m, 1H), 7.05 (s, 1H), 7.15 (m, 1H), 7.275 (m, 1H), 7.70 (d, 2H), 7.77 (d, 2H), 8.12 (s, 1H), 8.25 (s, 1H), 8.50 (bs, 1H), 9.52 (s, 1H) MS: MH⁺ 409 HPLC: 100%

### EXAMPLE 75

### 4-[4-(4-Methylsulphonylphenyl)phenyl]-N-(2-morpholin-4-ylethyl)thiophene2-carboxamide

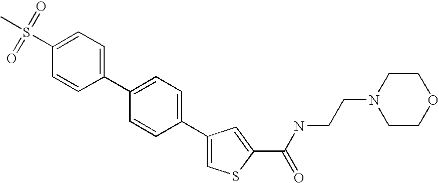

The product (149.5 mg) is obtained according to the process of Example 72, using [4-(methylsulphonyl)phenyl]boronic acid as cosubstrate.

Yield: 98% ¹H NMR (DMSO) δ (ppm): 2.45 (m, 4H), 2.50 (m, 2H), 3.27 (s, 3H), 3.40 (m, 2H), 3.60 (m, 4H), 7.85 (m, 4H), 8.05 (s, 4H), 8.20 (s, 1H), 8.30 (s, 1H), 8.55 (bs, 1H) MS: MH⁺ 471 HPLC: 98.1%

### EXAMPLE 76

### 4-[4-(3-Acetylphenyl)phenyl]-N2-morpholin4-ylethyl)thiophene-2-carboxamide

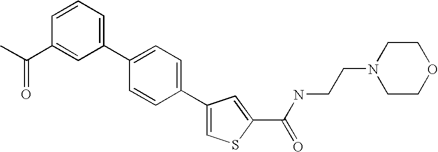

The product (113.6 mg) is obtained according to the process of Example 72, using (3-acetylphenyl)boronic acid as cosubstrate.

Yield: 99% ¹H NMR (DMSO) δ (ppm): 2.45 (m, 4H), 2.50 (m, 2H), 2.70 (s, 3H), 3.40 (m, 2H), 3.60 (m, 4H), 7.675 (m, 1H), 7.85 (m, 4H), 8.00 (m, 2H), 8.15 (s, 1H), 8.30 (m, 2H), 8.55 (bs, 1H) MS: MH⁺ 435 HPLC: 98.1%

### EXAMPLE 77

### N-(2-Morpholin-4-ylethyl)-4-[4-(trifluoromethoxy)phenyl]thiophene-2-carbothioamide

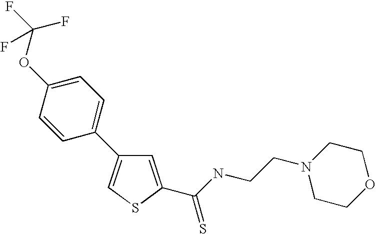

151 mg (1.5 equivalents) of Lawesson's reagent are added to a solution of 100 mg of the product of Example 5 in 4 ml of anhydrous toluene. The reaction medium is brought to 60° C. for 24 hours. After returning to ambient temperature, the crude reaction mixture is hydrolysed and extracted with ethyl acetate. The organic phases are combined, dried over sodium sulphate and concentrated under vacuum to yield to a yellow oil, which is purified on silica eluted with a dichloromethane/methanol gradient (from 100/0 to 90/10 in 5% increments). The purest fraction is taken up in an ether/pentane system and results, by precipitation, in the formation of yellow needles corresponding to the expected product (9 mg).

Yield: 9% ¹H NMR (DMSO) δ ppm: 2.45-2.64 (m, 6H), 3.58 (m, 4H), 3.86 (m, 2H), 7.47 (m, 2H), 7.80 (m, 2H), 8.15 (m, 2H), 10.17 (s, 1H) MS: MH⁺ 417 HPLC: 99.5%

### EXAMPLE 78

### N-(2-Morpholin-4-ylethyl)-4-[4-(1,2,3-thiadiazol-4-yl)phenyl]thiophene-2-carboxamide

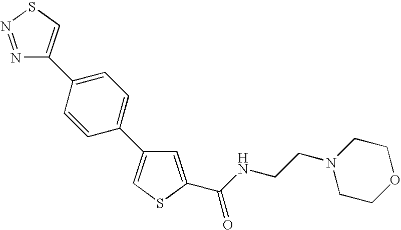

The product (21.7 mg) is obtained according to the process of Example 20, using 4-(4-bromophenyl)-1,2,3-thiadiazole as cosubstrate.

Yield: 14.5% ¹H NMR (DMSO) δ (ppm): 2.42 (m, 4H), 2.50 (m, 2H), 3.40 (m, 2H), 3.60 (m, 4H), 7.90 (d, 2H), 8.25 (m, 3H), 8.30 (s, 1H), 8.55 (bs, 1H), 9.70 (s, 1H) MS: MH⁺ 401 HPLC: 99.3%

### EXAMPLE 79

### 4-(4-Isoxazol-5-ylphenyl)-N-(2-morpholin-4-ylethyl)thiophene-2-carboxamide

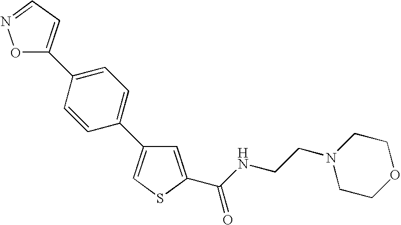

The product (32.1 mg) is obtained according to the process of Example 20, using 5-(4-bromophenyl)isoxazole as cosubstrate.

Yield: 22.5% ¹H NMR (CDCl₃) δ (ppm): 2.45 (m, 4H), 2.55 (m, 2H), 3.50 (m, 2H), 3.70 (m, 4H), 6.47 (s, 1H), 6.60 (bs, 1H), 7.55 (s, 1H), 7.62 (d, 2H), 7.72 (s, 1H), 7.80 (d, 2H), 8.25 (s, 1H) MS: MH⁺ 384 HPLC: 100%

### EXAMPLE 80

### trans-[4-([4-(4-Trifluoromethoxyphenyl)thien-2-yl]carboxamido)-cyclohexyl]acetic acid

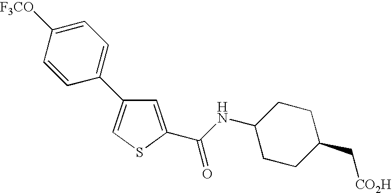

### Stage 1: Ethyl trans-[4-([4-(4-trifluoromethoxyphenyl)thien-2-yl]carboxamido)-cyclohexyl]acetate

0.15 g of ethyl trans-(4-aminocyclohexyl)acetate prepared according to the method described in J. Med. Chem., 1998, 41, 760-771, (1.1 equivalents), 365 mg of O-[(ethoxycarbonyl)cyanomethyleneamino]-N,N,N′,N′-tetramethyluronium tetrafluoroborate (TOTU) and 420 μl of N-ethyl-N,N-diisopropylamine are added to a solution of 229 mg of the compound obtained in Stage 2 of Preparation 5 in 4.0 ml of anhydrous dimethylformamide. The reaction medium is stirred at ambient temperature for 17 hours and is then concentrated under reduced pressure. The residue obtained is dissolved in ethyl acetate (50 ml), washed with water (2×20 ml), dried over sodium sulphate, filtered and concentrated under reduced pressure. Chromatography of the residue on silica gel (cyclohexane/ethyl acetate: 70/30) makes it possible to isolate 62.5 mg of the expected product.

Yield: 17% MS: MH⁺ 456 HPLC: 100%

### Stage 2: trans-[4-([4-(4-Trifluoromethoxyphenyl)thien-2-yl]carboxamido)-cyclohexy]acetic acid

The product (62.0 mg) is obtained according to the process of Example 27, using the product obtained in Stage 1 as substrate.

Yield: 100% ¹H NMR (DMSO) δ (ppm): 12.0 (s, 1H), 8.35 (bs, 1H), 8.25 (m, 1H), 8.10 (s, 11H), 7.8 (m, 2H), 7.45 (m, 2H), 3.75 (m, 1H), 2.25 (m, 2H), 2.0 (m, 1H), 1.8 (m, 2H), 1.7 (m, 2H), 1.5 (m, 1H), 1.40 (m, 2H), 1.10 (m, 2H) MS: MH⁺ 428, 472 (+HCOOH) HPLC: 100%

### EXAMPLE 81

### 2-[4-({[4-(4-Phenoxyphenyl)thien-2-yl]carboxamido}methyl)phenyl]-acetic acid

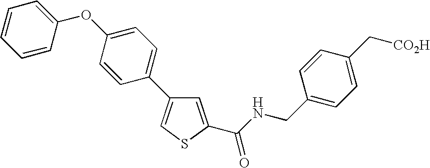

### Stage 1: Ethyl 2-(4-{[(4-bromothien-2-yl)carboxamido]methyl}phenyl)acetate

The product (135.0 mg) is obtained according to the process of Preparation 17, using the product obtained in Preparation 11 as substrate.

Yield: 49%

MS: MH⁺ 383

### Stage 2: Ethyl 2-(4-[{[4-(4-phenoxyphenyl)thien-2-yl]carboxamido}methyl]phenyl)-acetate

The product (164.0 mg) is obtained according to the process of Example 1, using the product obtained in Stage 1 as substrate and (4-phenoxyphenyl)boronic acid as cosubstrate.

Yield: 58% HPLC: 100%

### Stage 3: 2-[4-([{4-(4-Phenoxyphenyl)thien-2-yl]carboxamido}methyl)phenyl]acetic acid

The product (122.0 mg) is obtained according to the process of Example 27, using the product obtained in Stage 2 as substrate.

Yield: 97% ¹H NMR (DMSO) δ (ppm): 9.1 (bs, 1H), 8.30 (s, 1H), 8.25 (m, 1H), 8.00 (s, 1H), 7.8 (m, 2H), 7.45 (m, 2H), 7.3 (m, 4H), 7.15 (m, 1H), 7.0 (m, 4H), 4.5 (d, 2H), 3.5 (s, 2H) MS: MH⁻ 528 HPLC: 100%

### EXAMPLE 82

### 4-(4-Trifluoromethoxyphenyl)-N-methylthiophene-2-carboxamide

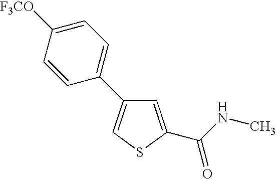

The product (112.9 mg) is obtained according to the process of Stage 1 of Example 80, using the product obtained in Stage 2 of Preparation 5 as substrate and methylamine as cosubstrate.

Yield: 43% ¹H NMR (DMSO) δ (ppm): 8.60 (bs, 1H), 8.15 (s, 1H), 8.10 (s, 1H), 7.80 (m, 2H), 7.45 (m, 2H), 2.80 (d, 3H) MS: MW⁺ 302, 346 (+HCOOH) HPLC: 100%

### EXAMPLE 83

### 2-[4-({[4-(4-Benzyloxyphenyl)thien-2-yl]carboxamido}methyl)phenyl]acetic acid

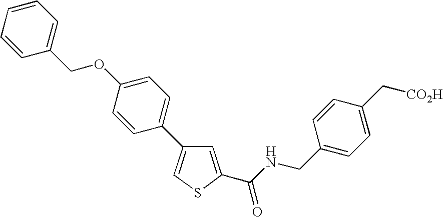

### Stage 1: Ethyl 2-[4-([{4-(4-benzyloxyphenyl)thien-2-yl]carboxamido}methyl)phenyl]-acetate

The product (45.0 mg) is obtained according to the process of Example 1, using the product obtained in Stage 1 of Example 81 as substrate and (4-benzyloxyphenyl)boronic acid as cosubstrate.

Yield: 47% HPLC: 100%

### Stage 2: 2-[4-({[4-(4-Benzyloxyphenyl)thien-2-yl]carboxamido}methyl)phenyl]acetic acid

The product (40.0 mg) is obtained according to the process of Example 27, using the product obtained in Stage 2 as substrate.

Yield: 95% ¹H NMR (DMSO) δ (ppm): 12.25 (m, 1H), 9.1 (bs, 1H), 8.25 (s, 1H), 8.0 (s, 1H), 7.6 (m, 2H), 7.5 (m, 2H), 7.45 (m, 2H), 7.3 (m, 1H), 7.2 (m, 2H), 7.15 (m, 2H), 7.0 (m, 2H), 5.2 (s, 2H), 4.4 (d, 2H), 3.5 (s, 2H) HPLC: 96.6%

### EXAMPLE 84

### N-(3,5-Difluoro-4-hydroxybenzyl)-4-(4-trifluoromethoxyphenyl)-thiophene-2-carboxamide

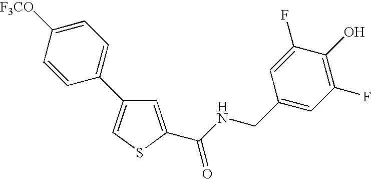

### Stage 1: 4-(Aminomethyl)-2,6-difluorophenol

The product (409.0 mg) is obtained by applying the process described in Stages 2 and 3 of Preparation 8, using 4-bromo-2,6-difluorophenol as substrate.

Yield: 74% MS: MH⁺ 160, MH⁻ 158 HPLC: 100%

### Stage 2: N-(3,5-Difluoro-4-hydroxybenzyl)-4-(4-trifluoromethoxyphenyl)thiophene-2-carboxamide

The product (49.0 mg) is obtained according to the process of Stage 1 of Example 80, using the product obtained in Stage 1 as substrate and the product obtained in Stage 2 of Preparation 5 as cosubstrate.

Yield: 42% ¹H NMR (DMSO) δ (ppm): 10.25 (s, 1H), 9.1 (bs, 1H), 8.25 (s, 1H), 8.0 (s, 1H), 7.8 (m, 2H), 7.5 (m, 2H), 7.0 (m, 2H), 4.4 (d, 2H) HPLC: 100% MS: MH⁺ 430, MH⁻ 428

### EXAMPLE 85

### 4-[4-(3-Nitrophenyl)phenyl]-N-(2-morpholin-4-ylethyl)thiophene-2-carboxamide

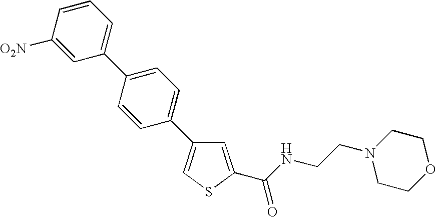

The product (66.2 mg) is obtained according to the process of Example 72, using (3-nitrophenyl)boronic acid as cosubstrate.

Yield: 59.8% ¹H NMR (DMSO) δ (ppm): 2.45 (m, 4H), 2.50 (m, 2H), 3.40 (m, 2H), 3.60 (m, 4H), 7.80 (m, 1H), 7.85 (m, 2H), 7.92 (m, 2H), 8.22 (m, 3H), 8.30 (s, 1H), 8.52 (m, 2H) MS: MH⁺ 438 HPLC: 99.2%

### EXAMPLE 86

### 4-[4-(2-Chlorophenyl)phenyl]-N-(2-morpholin-4-ylethyl)thiophene-2-carboxamide

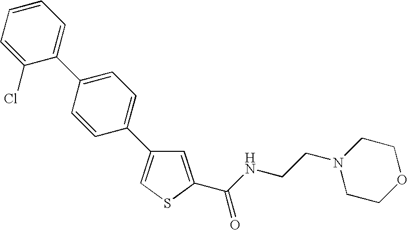

The product (36.3 mg) is obtained according to the process of Example 72, using (2-chlorophenyl)boronic acid as cosubstrate.

Yield: 33.6% ¹H NMR (DMSO) δ (ppm): 2.45 (m, 4H), 2.50 (m, 2H), 3.40 (m, 2H), 3.60 (m, 4H), 7.45 (m, 3H), 7.52 (d, 2H), 7.60 (m, 1H), 7.80 (d, 2H), 8.15 (s, 1H), 8.27 (s, 1H), 8.50 (bs, 1H) MS: MH⁺ 427.5 HPLC: 97.7%

### EXAMPLE 87

### 4-[4-(3-Cyanophenyl)phenyl]-N-(2-morpholin-4-ylethyl)thiophene-2-carboxamide

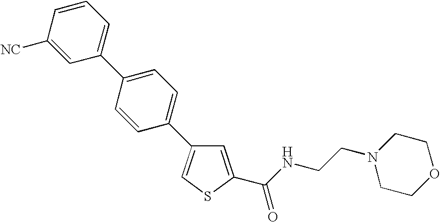

The product (66.4 mg) is obtained according to the process of Example 72, using (3-cyanophenyl)boronic acid as cosubstrate.

Yield: 62.9% ¹H NMR (DMSO) δ (ppm): 2.45 (m, 4H), 2.50 (m, 2H), 3.40 (m, 2H), 3.60 (m, 4H), 7.67 (m, 1H), 7.85 (m, 5H), 8.10 (m, 1H), 8.20 (s, 1H), 8.25 (s, 1H), 8.30 (s, 1H), 8.50 (bs, 1H) MS: MH⁺ 418 HPLC: 98.3%

### EXAMPLE 88

### 4-(4-(2-Formylphenyl)phenyl]-N-(2-morpholin-4-ylethyl)thiophene-2-carboxamide

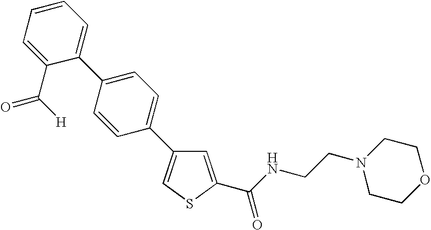

The product (61.9 mg) is obtained according to the process of Example 72, using (2-formylphenyl)boronic acid as cosubstrate.

Yield: 58.2% ¹H NMR (DMSO) δ (ppm): 2.45 (m, 4H), 2.50 (m, 2H), 3.40 (m, 2H), 3.60 (m, 4H), 7.57 (m, 4H), 7.77 (m, 1H), 7.82 (d, 2H), 7.95 (m, 1H), 8.20 (s, 1H), 8.30 (s, 1H), 8.52 (bs, 1H), 9.95 (s, 1H) MS: MH⁺ 421 HPLC: 97.5%

### EXAMPLE 89

### 4-[4-(3,4,5-Trimethoxyphenyl)phenyl]-N-(2-morpholin-4-ylethyl)thiophene-2-carboxamide

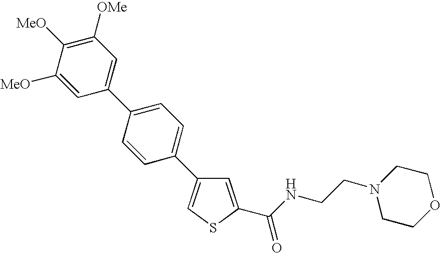

The product (77.4 mg) is obtained according to the process of Example 72, using (3,4,5-trimethoxyphenyl)boronic acid as cosubstrate.

Yield: 63.4% ¹H NMR (DMSO) δ (ppm): 2.45 (m, 4H), 2.50 (m, 2H), 3.40 (m, 2H), 3.60 (m, 4H), 3.70 (s, 3H), 3.87 (s, 6H), 6.95 (s, 2H), 7.75 (s, 4H), 8.12 (s, 1H), 8.25 (s, 1H), 8.50 (bs, 1H) MS: MH⁺ 483 HPLC: 100%

### EXAMPLE 90

### 4-{4-[4-(Hydroxymethyl)phenyl]phenyl}-N-(2-morpholin-4-ylethyl)thiophene-2-carboxamide

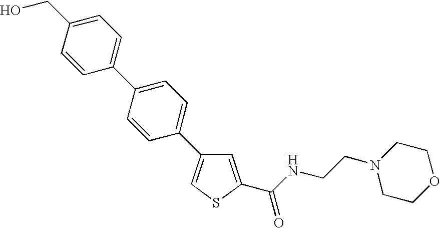

The product (82.9 mg) is obtained according to the process of Example 72, using [4-(hydroxymethyl)phenyl]boronic acid as cosubstrate.

Yield: 77.6% ¹H NMR (DMSO) δ (ppm): 2.45 (m, 4H), 2.50 (m, 2H), 3.40 (m, 2H), 3.60 (m, 4H), 4.55 (s, 2H), 5.22 (bs, 1H), 7.40 (d, 2H), 7.70 (d, 2H), 7.77 (s, 4H), 8.10 (s, 1H), 8.25 (s, 1H), 8.50 (bs, 1H) MS: MH⁺ 423 HPLC: 100%

### EXAMPLE 91

### 4-[4-(3-Acetamidophenyl)phenyl]-N-(2-morpholin-4-ylethyl)thiophene-2-carboxamide

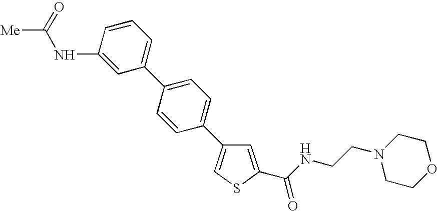

The product (100.3 mg) is obtained according to the process of Example 72, using (3-acetamidophenyl)boronic acid as cosubstrate.

Yield: 88.2% ¹H NMR (DMSO) δ (ppm): 2.45 (m, 4H), 2.50 (m, 2H), 3.40 (m, 2H), 3.60 (m, 4H), 7.40 (m, 2H), 7.57 (m, 1H), 7.70 (d, 2H), 7.80 (d, 2H), 7.95 (s, 1H), 8.15 (s, 1H), 8.25 (s, 1H), 8.55 (bs, 1H), 10.05 (bs, 1H) MS: MH⁺ 451 HPLC: 100%

### EXAMPLE 92

### 4-{4-[3,4-(Methylenedioxy)phenyl]phenyl}-N-(2-morpholin-4-ylethyl)thiophene-2-carboxamide

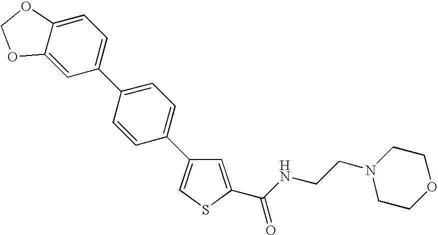

The product (121.4 mg) is obtained according to the process of Example 72, using [3,4-(methylenedioxy)phenyl]boronic acid as cosubstrate.

Yield: 99.2% ¹H NMR (DMSO) δ (ppm): 2.45 (m, 4H), 2.50 (m, 2H), 3.40 (m, 2H), 3.60 (m, 4H), 6.175 (s, 2H), 7.00 (m, 1H), 7.22 (m, 1H), 7.32 (s, 1H), 7.75 (m, 4H), 8.10 (s, 1H), 8.52 (bs, 1H) MS: MH⁺ 437 HPLC: 96.9%

### EXAMPLE 93

### N-(Cyclopropylmethyl)4-[4-(trifluoromethoxy)phenyl]thiophene-2-carboxamide

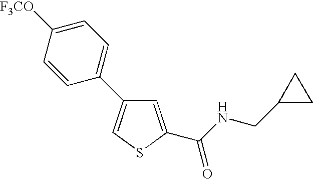

The product (146.0 mg) is obtained according to the process of Example 80, Stage 1, using the product obtained in Stage 2 of Preparation 5 as cosubstrate and cyclopropylmethylamine.

Yield: 41% ¹H NMR (CDCl₃) δ (ppm): 7.8 (s, 1H), 7.6 (m, 2H), 7.5 (s, 1H), 7.3 (m, 2H), 6.1 (bs, 1H), 3.3 (m, 2H), 1.2 (m, 1H), 0.8 (m, 2H), 0.3 (m, 2H) MS: MH⁺ 342, 386 (+HCOOH) HPLC: 100%

### EXAMPLE 94

### N-(tert-Butyl)4-[4-(trifluoromethoxy)phenyl]thiophene-2-carboxamide

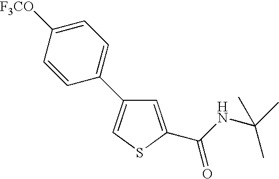

The product (171.0 mg) is obtained according to the process of Example 80, Stage 1, using the product obtained in Stage 2 of Preparation 5 as cosubstrate and tert-butylamine.

Yield: 47.8% ¹H NMR (CDCl₃) δ (ppm): 7.7 (s, 1H), 7.6 (m, 2H), 7.5 (s, 1H), 7.3 (m, 2H), 5.9 (bs, 1H), 1.5 (s, 9H) MS: MH⁺ 344, 388 (+HCOOH) HPLC: 100%

### EXAMPLE 95

### N-(Cyclopropyl)-4-[4-trifluoromethoxy)phenyl]thiophene-2-carboxamide

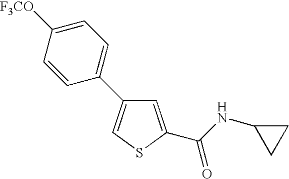

The product (168.0 mg) is obtained according to the process of Example 80, Stage 1, using the product obtained in Stage 2 of Preparation 5 as cosubstrate and cyclopropylamine.

Yield: 49.3% ¹H NMR (CDCl₃) δ (ppm): 7.8 (s, 1H), 7.6 (m, 3H), 7.3 (m, 1H), 6.1 (bs, 1H), 0.9 (m, 2H), 0.7 (m, 2H) MS: MH⁺ 328, 362 (+HCOOH) HPLC: 100%

### EXAMPLE 96

### N-(Cyclopentyl)-4-[(4(trifluoromethoxy)phenyl]thiophene-2-carboxamide

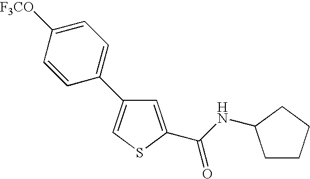

The product (281.0 mg) is obtained according to the process of Example 80, Stage 1, using the product obtained in Stage 2 of Preparation 5 as cosubstrate and cyclopentylamine.

Yield: 76% ¹H NMR (CDCl₃) δ (ppm): 7.8 (s, 1H), 7.6 (m, 2H), 7.5 (s, 1H), 7.3 (m, 2H), 6.1 (bs, 1H), 4.5 (m, 1H), 2.2 (m, 2H), 1.8 (m, 4H), 1.7 (m, 2H) MS: MH⁺ 356, 400 (+HCOOH) HPLC: 100%

### EXAMPLE 97

### N-(2-Hydroxyethyl)-4-[4-(trifluoromethoxy)phenyl]thiophene-2-carboxamide

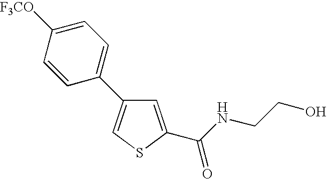

The product (171.0 mg) is obtained according to the process of Example 80, Stage 1, using the product obtained in Stage 2 of Preparation 5 as cosubstrate and 2-ethanolamine.

Yield: 49.5% ¹H NMR (CDCl₃) δ (ppm): 8.8 (bs, 1H), 8.2 (s, 1H), 8.1 (s, 1H), 7.8 (m, 2H), 7.5 (m, 2H), 4.8 (m, 1H), 3.4 (m, 2H), 3.3 (m, 2H) MS: MH⁺ 332, 376 (+HCOOH) HPLC: 100%

### EXAMPLE 98

### N-(Cyclopentylmethyl)-4-[4-(trifluoromethoxy)phenyl]thiophene-2-carboxamide

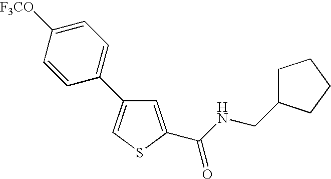

The product (95.6 mg) is obtained according to the process of Example 80, Stage 1, using the product obtained in Stage 2 of Preparation 5 as cosubstrate and 1-cyclopentylmethanamine.

Yield: 11% ¹H NMR (CDCl₃) δ (ppm): 7.8 (s, 1H), 7.6 (m, 2H), 7.5 (s, 1H), 7.2 (m, 2H), 6.1 (m, 1H), 3.4 (m, 2H), 2.2 (m, 1H), 1.8 (m, 2H), 1.7 (m, 2H), 1.6 (m, 2H), 1.3 (m, 2H) MS: MH⁺ 370, 414 (+HCOOH) HPLC: 100%

### EXAMPLE 99

### trans-4-({[4-(4-Trifluoromethoxyphenyl)thien-2-yl]carboxamido}-methyl)cyclohexanecarboxylic acid, Sodium Salt

50 mg of the compound of Example 35, in the presence of NaOH (1.0 equivalent) in 2 ml of methanol, are stirred at 0° C. for 15 minutes and then at 25° C. for 30 minutes. The reaction medium is concentrated under reduced pressure in order to produce 42 mg of the desired product.

Yield: 79% ¹H NMR (DMSO) δ (ppm): 8.4 (bs, 1H), 8.27 (s, 1H), 8.1 (s, 1H), 7.8 (m, 2H), 7.45 (m, 2H), 3.08 (m, 2H), 1.85 (m, 2H), 1.7 (m, 3H), 1.45 (m, 1H), 1.15 (m, 2H), 0.85 (m, 2H) MS: MH⁺ 427 MH⁻ (+HCOOH) 472 HPLC: 100%

### EXAMPLE 100

### 4-{4-[(3-Chloro-4-fluoro)phenyl]phenyl}-N-(2-morpholin-4-ylethyl)-thiophene-2-carboxamide

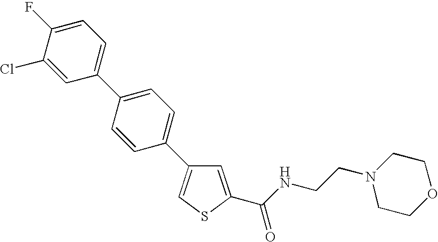

The product (35.7 mg) is obtained according to the process of Example 1, using the compound of Example 71 and [(3-chloro-4-fluoro)phenyl]boronic acid as substrates.

Yield: 31% ¹H NMR (DMSO) δ (ppm): 8.55 (bs, 1H), 8.27 (s, 1H), 8.17 (s, 1H), 7.95 (m, 1H), 7.8 (m, 5H), 7.5 (m, 1H), 3.6 (m, 4H), 3.4 (m, 2H), 2.5 (m, 2H), 2.45 (m, 4H) MS: MH⁺ 445 HPLC: 98.1%

### EXAMPLE 101

### 4-{4-[3-(Hydroxymethyl)phenyl]phenyl}-N-(2-morpholin-4-ylethyl)-thiophene-2-carboxamide

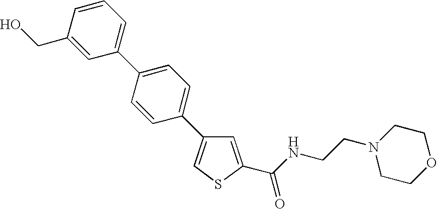

The product (172.8 mg) is obtained according to the process of Example 1, using the compound of Example 71 and [3-(hydroxymethyl)phenyl]boronic acid as substrates.

Yield: 64% ¹H NMR (DMSO) δ (ppm): 8.5 (bs, 1H), 8.25 (s, 1H), 8.15 (s, 1H), 7.77 (m, 4H), 7.67 (m, 1H), 7.60 (m, 1H), 7.45 (m, 1H), 7.35 (m, 1H), 5.25 (t, 1H), 4.60 (d, 2H), 3.6 (m, 4H), 3.4 (m, 2H), 2.5 (m, 2H), 2.45 (m, 4H) MS: MH⁺ 423 HPLC: 97.0%

### EXAMPLE 102

### Ethyl 2-[4-({[4-(4′-propionyl-4-biphenyl)thien-2-yl]carboxamido}-methyl)phenyl]acetate

### Stage 1: Ethyl 2-[4-({[4-(4-bromophenyl)thien-2-yl]carboxamido}methyl)phenyl]-acetate

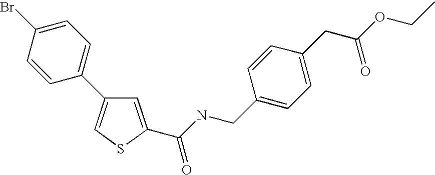

The product (16.37 g) is obtained according to the process of Stage 2 of Preparation 1, using the compound obtained in Stage 2 of Example 71 and the compound obtained in Preparation 11 as substrates.

Yield: 75% ¹H NMR (CDCl₃) δ (ppm): 7.65 (s, 1H), 7.50 (s, 1H), 7.45 (m, 2H), 7.30 (m, 2H), 7.25 (m, 2H), 7.20 (m, 2H), 6.30 (bs, 1H), 4.50 (m, 2H), 4.10 (q, 2H), 3.55 (s, 2H), 1.20 (t, 3H)

### Stage 2: Ethyl 2-{4-[({4-[4-(pinacolboro)phenyl]thien-2-yl}carboxamido)methyl]-phenyl}acetate

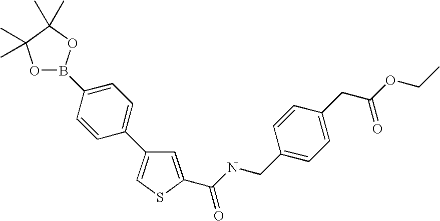

The product (19.5 g) is obtained according to the process of Stage 4 of Example 18, methyl 4-(4-{[(trifluoromethyl)sulphonyl]oxy}phenyl)thiophene-2-carboxylate being replaced with the product obtained in the preceding Stage 1.

Yield: 98% ¹H NMR (CDCl₃) δ (ppm): 7.80 (m, 2H), 7.75 (s, 1H), 7.55 (s, 1H), 7.50 (m, 2H), 7.25 (m, 2H), 7.20 (m, 2H), 6.20 (bs, 1H), 4.55 (m, 2H), 4.10 (q, 2H), 3.55 (s, 2H), 1.25 (s, 12H), 1.15 (t, 3H)

### Stage 3: Ethyl {[({4-[4-(dihydroxyboro)phenyl]thien-2-yl}carboxamido)methyl]-phenyl}acetate

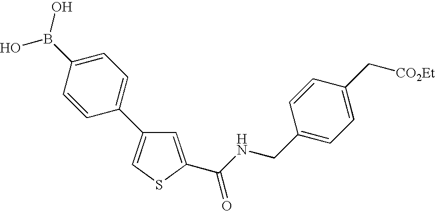

The product (12.8 g) is obtained according to the process of Stage 5 of Example 18, using the compound obtained in the preceding Stage 2.

Yield: 78% ¹H NMR (DMSO) δ (ppm): 9.05 (bs, 1H), 8.30 (s, 1H), 8.10 (s, 1H), 8.05 (s, 2H), 7.85 (m, 2H), 7.65 (m, 2H), 7.30 (m, 2H), 7.20 (m, 2H), 4.45 (m, 2H), 4.05 (q, 2H), 3.65 (s, 2H), 1.15 (t, 3H)

### Stage 4: Ethyl 2-[4-({[4-(4′-propionyl-4-biphenyl)thien-2-yl]carboxamido}-methyl)phenyl]acetate

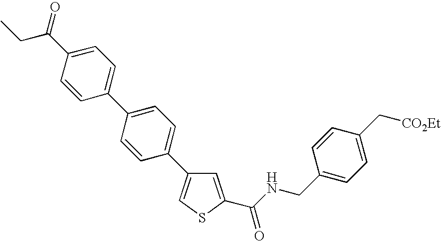

The product (157.9 mg) is obtained according to the process of Stage 1 of Preparation 5, using the compound obtained in the preceding Stage 3 and 1-(4-bromophenyl)propan-1-one as substrates.

Yield: 87% ¹H NMR (DMSO) δ (ppm): 9.10 (bs, 1H), 8.35 (s, 1H), 8.20 (s, 1H), 8.05 (m, 2H), 8.00 (m, 2H), 7.85 (m, 4H), 7.25 (m, 4H), 4.45 (m, 2H), 4.05 (q, 2H), 3.65 (s, 2H), 3.10 (q, 2H), 1.20 (t, 3H), 1.10 (t, 3H) HPLC: 99.7%

### EXAMPLE 103

### 2-[4-({[4-(4′-Propionyl-4-biphenyl)thien-2-yl]carboxamido}-methyl)phenyl]acetic acid

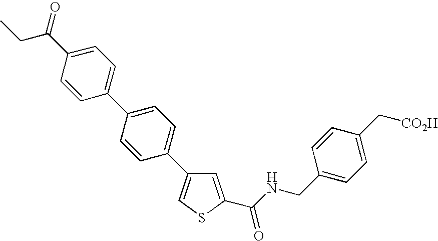

The product (80 mg) is obtained according to the process of Example 27, using the compound obtained in Example 102 as substrate.

Yield: 53% ¹H NMR (DMSO) δ (ppm): 12.25 (bs, 1H), 9.12 (bs, 1H), 8.4 (s, 1H), 8.2 (s, 1H), 8.05 (m, 2H), 7.85 (m, 6H), 7.15 (m, 4H), 4.45 (m, 2H), 3.2 (m, 2H), 3.1 (m, 2H), 1.1 (m, 3H) HPLC: 98.9%

### EXAMPLE 104

### Ethyl 2-[4-({[4-(4′-cyclopropylcarbonyl-4-biphenyl)thien-2-yl]-carboxamido}methyl)phenyl]acetate

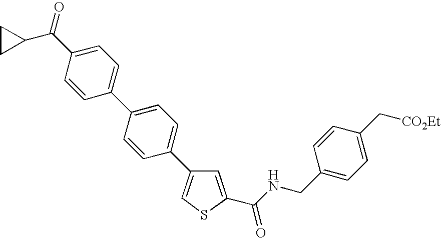

The product (107.4 mg) is obtained according to the process of Stage 4 of Example 102, using (4-bromophenyl)(cyclopropyl)methanone in place of 1-(4-bromophenyl)propan-1-one.

Yield: 57% ¹H NMR (DMSO) δ (ppm): 9.10 (bs, 1H), 8.35 (s, 1H), 8.20 (s, 1H), 8.15 (m, 2H), 7.90 (m, 2H), 7.85 (m, 6H), 7.30 (m, 2H), 7.25 (m, 2H), 4.45 (m, 2H), 4.05 (q, 2H), 3.60 (s, 2H), 2.95 (m, 1H), 1.15 (t, 3H), 1.05 (m, 4H) HPLC: 96.8%

### EXAMPLE 105

### 2-[4-({[4-(4′-Cyclopropylcarbonyl-4-biphenyl)thien-2-yl]-carboxamido}methyl)phenyl]acetic acid

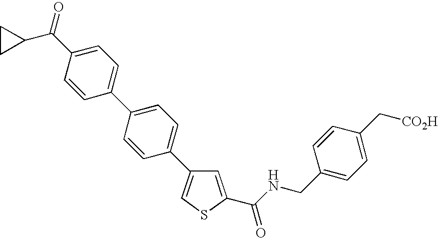

The product (77.5 mg) is obtained according to the process of Example 27, using the compound obtained in Example 104 as substrate.

Yield: 76% ¹H NMR (DMSO) δ (ppm): 12.25 (bs, 1H), 9.1 (bs, 1H), 8.4 (s, 1H), 8.15 (s, 1H), 8.1 (m, 2H), 7.9 (m, 2H), 7.85 (m, 4H), 7.17 (s, 4H), 4.4 (m, 2H), 3.2 (m, 2H), 2.95 (m, 1H), 1.05 (m, 4H) HPLC: 98.9%

### EXAMPLE 106

### Ethyl 2-[4-({[4-(4′-benzoyl-4-biphenyl)thien-2-yl]carboxamido}-methyl)phenyl]acetate

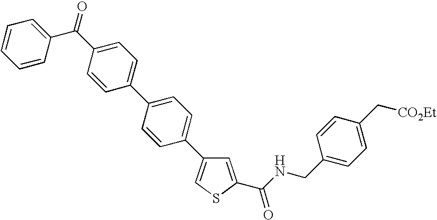

The product (167.0 mg) is obtained according to the process of Stage 4 of Example 102, using (4-bromophenyl)(phenyl)methanone in place of 1-(4-bromophenyl)propan-1-one.

Yield: 84% ¹H NMR (DMSO) δ (ppm): 9.05 (bs, 1H), 8.35 (s, 1H), 8.20 (s, 1H), 7.95 (m, 2H), 7.85 (m, 6H), 7.80 (m, 2H), 7.70 (m, 1H), 7.60 (m, 2H), 7.30 (m, 2H), 7.25 (m, 2H), 4.45 (m, 2H), 4.05 (q, 2H), 3.65 (s, 2H), 1.15 (t, 3H) HPLC: 99.4%

### EXAMPLE 107

### 2-[4-({[4-(4′-Benzoyl-4-biphenyl)thien-2-yl]carboxamido}-methyl)phenyl]acetic acid

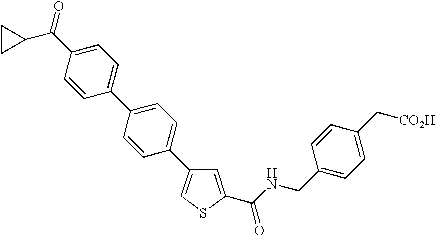

The product (124.3 mg) is obtained according to the process of Example 27, using the compound obtained in Example 106 as substrate.

Yield: 78% ¹H NMR (DMSO) δ (ppm): 12.25 (bs, 1H), 9.1 (bs, 1H), 8.4 (s, 1H), 8.2 (s, 1H), 7.95 (m, 2H), 7.85 (m, 6H), 7.80 (m, 2H), 7.7 (m, 1H), 7.6 (m, 2H), 7.2 (s, 4H), 4.45 (m, 2H), 3.2 (m, 2H) HPLC: 99.3%

### EXAMPLE 108

### Ethyl 2-[4-({[4-(4′-cyanomethyl-4-biphenyl)thien-2-yl]carboxamido}-methyl)phenyl]acetate

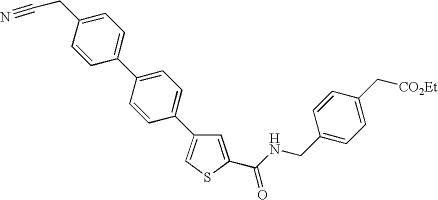

The product (91.9 mg) is obtained according to the process of Stage 4 of Example 102, using (4-bromophenyl)acetonitrile in place of 1-(4-bromophenyl)propan-1-one.

Yield: 52% ¹H NMR (DMSO) δ (ppm): 9.05 (bs, 1H), 8.35 (s, 1H), 8.15 (s, 1H), 7.75 (m, 6H), 7.45 (m, 2H), 7.30 (m, 2H), 7.25 (m, 2H), 4.45 (m, 2H), 4.10 (m, 6H), 3.65 (m, 2H), 1.15 (t, 3H) HPLC: 94.9%

### EXAMPLE 109

### 2-[4-({[4-(4′-Cyanomethyl-4-biphenyl)thien-2-yl]carboxamido}-methyl)phenyl]acetic acid

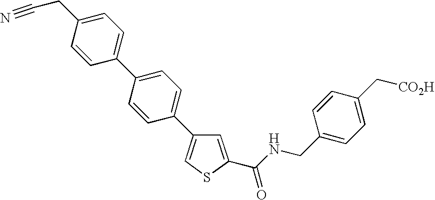

The product (38.2 mg) is obtained according to the process of Example 27, using the compound obtained in Example 108 as substrate.

Yield: 45% ¹H NMR (DMSO) δ (ppm): 12.25 (bs, 1H), 9.05 (bs, 1H), 8.35 (s, 1H), 8.15 (s, 1H), 7.77 (m, 6H), 7.45 (m, 2H), 7.25 (m, 4H), 4.45 (m, 2H), 4.1 (s, 2H), 3.45 (s, 2H) MS: MH⁺ 467 HPLC: 96.9%

### EXAMPLE 110

### Ethyl 2-{4-[({4-4-(pyrimidin-5-yl)phenyl]thien-2-yl}carboxamido)-methyl]phenyl}acetate

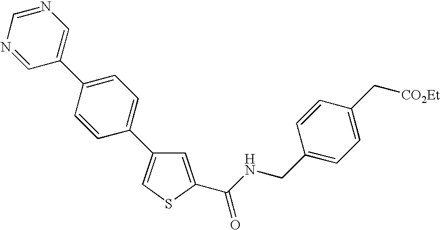

The product (78.5 mg) is obtained according to the process of Stage 4 of Example 102, using 5-bromopyrimidine in place of 1-(4-bromophenyl)propan-1-one.

Yield: 48% ¹H NMR (DMSO) δ (ppm): 9.20 (m, 3H), 9.05 (bs, 1H), 8.35 (s, 1H), 8.25 (s, 1H), 7.95 (m, 2H), 7.85 (m, 2H), 7.30 (m, 2H), 7.25 (m, 2H), 4.45 (m, 2H), 4.05 (q, 2H), 3.65 (s, 2H), 1.20 (t, 3H) HPLC: 97.8%

### EXAMPLE 111

### 2-{4-[({4-[4-(Pyrimidin-5-yl)phenyl]thien-2-yl}carboxamido)-methyl]phenyl}acetic acid

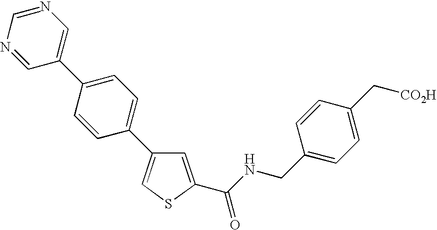

The product (17.8 mg) is obtained according to the process of Example 27, using the compound obtained in Example 110 as substrate.

Yield: 24% ¹H NMR (DMSO) δ (ppm): 12.25 (bs, 1H), 9.2 (m, 3H), 9.05 (bs, 1H), 8.35 (s, 1H), 8.2 (s, 1H), 7.92 (d, 2H), 7.85 (d, 2H), 7.3 (d, 2H), 7.25 (d, 2H), 4.45 (m, 2H), 3.55 (s, 2H) MS: MH⁺ 430 HPLC: 99.1%

### EXAMPLE 112

### Ethyl 2-{4-[({4-[4-(4-acetyl-3-hydroxyphenyl)phenyl]thien-2-yl}-carboxamido)methyl]phenyl}acetate

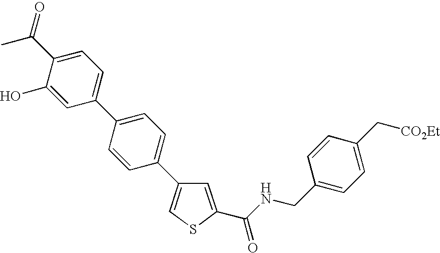

The product (154.2 mg) is obtained according to the process of Stage 4 of Example 102, using 4-bromo-2-hydroxyacetophenone, prepared according to Bioorg. Med. Chem., vol. 5, No. 2, p. 445-459, 1997, in place of 1-(4-bromophenyl)propan-1-one.

Yield: 63% ¹H NMR (DMSO) δ (ppm): 12.15 (bs, 1H), 9.05 (bs, 1H), 8.35 (s, 1H), 8.20 (s, 1H), 8.00 (m, 1H), 7.85 (m, 4H), 7.30 (m, 6H), 4.45 (m, 2H), 4.10 (q, 2H), 3.65 (s, 1H), 2.65 (s, 3H), 1.15 (t, 3H) HPLC: 96.3%

### EXAMPLE 113

### 2-{4-[({4-[4-Acetyl-3-hydroxyphenyl)phenyl]thien-2-yl}-carboxamido)methyl]phenyl}acetic acid

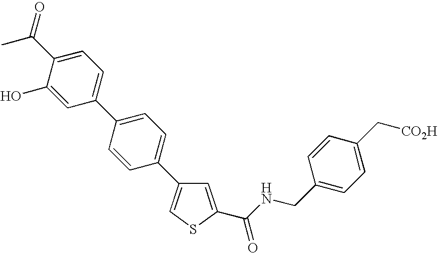

The product (59.3 mg) is obtained according to the process of Example 27, using the compound obtained in Example 112 as substrate.

Yield: 40% ¹H NMR (DMSO) δ (ppm): 12.25 (bs, 1H), 12.15 (bs, 1H), 9.05 (bs, 1H), 8.35 (s, 1H), 8.2 (s, 1H), 8.0 (m, 1H), 7.85 (m, 4H), 7.35 (m, 2H), 7.3 (m, 2H), 7.25 (m, 2H), 4.45 (m, 2H), 3.55 (s, 2H), 2.65 (s, 3H) HPLC: 98.1%

### EXAMPLE 114

### Ethyl 2-{4-[({4-[4-(2-hydroxyphenyl)phenyl]thien-2-yl}carbox-amido)methyl]phenyl}acetate

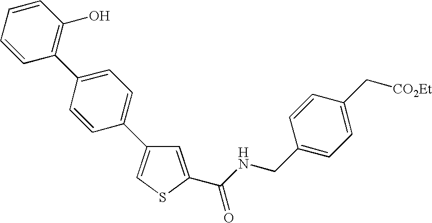

### Stage 1: Ethyl 2-{4-[({4-[4-(2-methoxymethoxyphenyl)phenyl]thien-2-yl}carboxamido)methyl]phenyl}acetate

The product is obtained according to the process of Stage 4 of Example 102, using 1-bromo-2-methoxymethoxyphenol in place of 1-(4-bromophenyl)propan-1-one.

Yield: 68%

### Stage 2: Ethyl 2-{4-[({4-[4-(2-hydroxyphenyl)phenyl]thien-2-yl}carbox-amido)methyl]phenyl}acetate

A solution of 369 mg of the compound obtained in the preceding Stage 1 in 4 ml of a 3.0M solution of hydrogen chloride in methanol is heated at 65° C. for 2 hours. The reaction medium is evaporated and then the residue obtained is dissolved in 50 ml of ethyl acetate. The organic phase is washed with water (2×20 ml), dried over sodium sulphate and then concentrated under reduced pressure. Chromatography of the residue on silica gel (dichloromethane/methanol: 97/3) makes it possible to isolate 230.9 mg of the expected product.

Yield: 68% ¹H NMR (DMSO) δ (ppm): 9.55 (s, 1H), 9.05 (bs, 1H), 8.30 (s, 1H), 8.10 (s, 1H), 7.70 (m, 2H), 7.60 (m, 2H), 7.30 (m, 3H), 7.25 (m, 2H), 7.15 (m, 1H), 6.95 (m, 1H), 6.85 (m, 1H), 4.45 (m, 2H), 3.65 (s, 2H), 3.60 (s, 3H) HPLC: 98.7%

### EXAMPLE 115

### 2-{4-[({4-[4-(2-Hydroxyphenyl)phenyl thien-2-yl}carboxamido)-methyl]phenyl}acetic acid

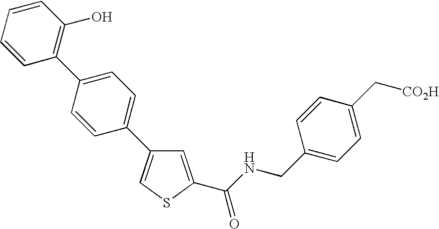

The product (178.3 mg) is obtained according to the process of Example 27, using the compound obtained in Example 114 as substrate.

Yield: 82% ¹H NMR (DMSO) δ (ppm): 12.3 (bs, 1H), 9.55 (bs, 1H), 9.05 (bs, 1H), 8.3 (s, 1H), 8.1 (s, 1H), 7.7 (d, 2H), 7.65 (d, 2H), 7.3 (m, 3H), 7.25 (m, 2H), 7.15 (m, 1H), 6.95 (m, 1H), 7.9 (m, 1H), 4.45 (m, 2H), 3.55 (s, 2H) MS: MH⁺ 444 HPLC: 95.9%

### EXAMPLE 116

### Ethyl 2-{4-[({4-[4-(4-acetyl-2-methoxyphenyl)phenyl]thien-2-yl}carboxamido)methyl]phenyl}acetate

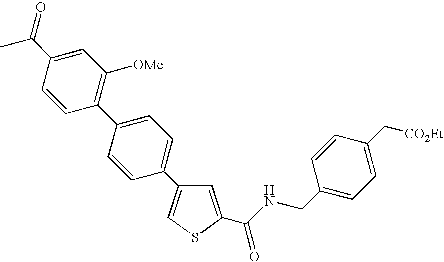

The product (530 mg) is obtained according to the process of Stage 4 of Example 102, using 4-acetyl-2-methoxyphenyl triflate, prepared from 4-hydroxy-3-methoxyacetophenone according to the process of Preparation 4, in place of 1-(4-bromophenyl)propan-1-one.

Yield: 66% ¹H NMR (DMSO) δ (ppm): 9.05 (bs, 1H), 8.35 (s, 1H), 8.15 (s, 1H), 7.75 (m, 2H), 7.70 (m, 1H), 7.60 (m, 3H), 7.50 (m, 1H), 7.25 (m, 4H), 4.45 (m, 2H), 4.05 (m, 2H), 3.85 (s, 3H), 3.65 (s, 2H), 2.65 (s, 3H) HPLC: 97.2%

### EXAMPLE 117

### 2-{4-[({4-[4-(4-Acetyl-2-hydroxyphenyl)phenyl]thien-2-yl}-carboxamido)methyl]phenyl}acetic acid

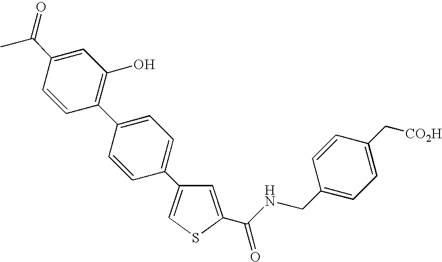

A solution of 277 mg of the compound obtained in Example 116 and of 110 mg of sodium ethanethiolate (2.5 equivalents) in 5.0 ml of dimethylformamide is heated at 130° C. for 2 hours. The reaction medium is concentrated under reduced pressure and then the residue obtained is dissolved in 100 ml of an ethyl acetate/1,2-dimethoxyethane (50/50) solution. The organic phase is washed with 30 ml of a 1.0M hydrochloric acid solution and then with water (3×30 ml), and finally dried over sodium sulphate and concentrated under reduced pressure. The residue obtained is triturated in 10 ml of acetonitrile. The insoluble material is subsequently filtered off and washed with ether (3×10 ml) to provide 51.2 mg of the expected product.

Yield: 20% ¹H NMR (DMSO) δ (ppm): 12.2 (bs, 1H), 10.05 (bs, 1H), 9.1 (bs, 1H), 8.35 (s, 1H), 8.15 (s, 1H), 7.75 (d, 2H), 7.7 (d, 2H), 7.5 (m, 3H), 7.3 (d, 2H), 7.22 (d, 2H), 4.45 (m, 2H), 3.55 (s, 2H), 2.55 (s, 3H) HPLC: 99.65%

### EXAMPLE 118

### Ethyl 2-(4-{[(4-{4-[4-(2-oxopropyl)2-methoxyphenyl]phenyl}thien-2-yl)carboxamido]methyl}phenyl)acetate

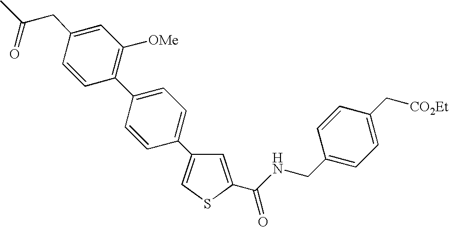

The product (390 mg) is obtained according to the process of Stage 4 of Example 102, using 4-(2-oxopropyl)-2-methoxyphenyl triflate, prepared from 1-(3-methoxy-4-hydroxyphenyl)propan-2-one according to the process of Preparation 4, in place of 1-(4-bromophenyl)propan-1-one.

Yield: 56% ¹H NMR (DMSO) δ (ppm): 9.05 (bs, 1H), 8.30 (s, 1H), 8.10 (s, 1H), 7.75 (m, 2H), 7.55 (m, 2H), 7.25 (m, 5H), 6.95 (s, 1H), 6.85 (m, 1H), 4.45 (m, 2H), 4.05 (q, 2H), 3.80 (s, 2H), 3.75 (s, 3H), 3.60 (s, 2H), 1.15 (t, 3H) HPLC: 96.4%

### EXAMPLE 119

### 2-(4-{[(4-{4-[4-(2-Oxopropyl)-3-hydroxyphenyl]phenyl}thien-2-yl)carboxamido]methyl}phenyl)acetic acid

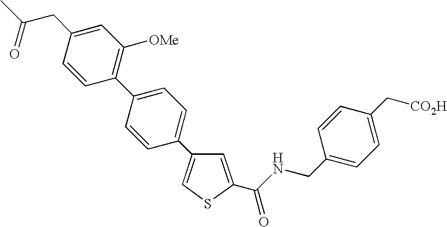

The product (128.1 mg) is obtained according to the process of Example 117 using the compound obtained in Example 118 as substrate.

Yield: 36% ¹H NMR (DMSO) δ (ppm): 12.3 (bs, 1H), 9.55 (s, 1H), 9.05 (bs, 1H), 8.3 (s, 1H), 8.1 (s, 1H), 7.7 (d, 2H), 7.65 (d, 2H), 7.3 (m, 5H), 6.70 (m, 2H), 4.50 (m, 2H), 3.70 (s, 2H), 3.60 (s, 2H), 2.15 (s, 3H) MS: MH⁺ 500 HPLC:100%

### EXAMPLE 120

### 4-[4-(2-Fluorophenyl)phenyl]-N-(2-morpholin-4-ylethyl)thiophene-2-carboxamide

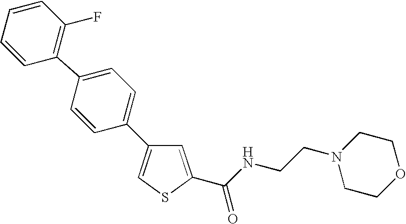

The product (179 mg) is obtained according to the process of Example 1, using the compound of Example 71 and (2-fluorophenyl)boronic acid as substrates.

Yield: 69% ¹H NMR (DMSO) δ (ppm): 8.5 (bs, 1H), 8.25 (s, 1H), 8.15 (s, 1H), 7.8 (m, 2H), 7.65 (m, 2H), 7.6 (m, 1H), 7.45 (m, 1H), 7.32 (m, 2H), 3.55 (m, 4H), 3.4 (m, 2H), 2.5 (m, 2H), 2.4 (m, 4H) MS: MH⁺ 411 HPLC: 97.4%

### EXAMPLE 121

### N-[2-(Hydroxymethyl)cyclohexyl]-4-(4-trifluoromethoxyphenyl)-thiophene-2-carboxamide

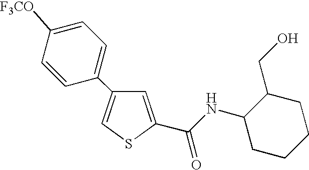

The product (240.3 mg) is obtained according to the process of Stage 3 of Example 6, using the compound obtained in Stage 2 of Preparation 5 and 2-(hydroxymethyl)cyclohexylamine hydrochloride as substrates.

Yield: 69% ¹H NMR (DMSO) δ (ppm): 8.35 (m, 2H), 8.15 (s, 1H), 7.8 (d, 2H), 7.45 (d, 2H), 4.32 (m, 1H), 3.6 (m, 1H), 3.45 (m, 1H), 3.25 (m, 1H), 1.9 (m, 2H), 1.7 (m, 2H), 1.5 (m, 1H), 1.25 (m, 4H) MS: MH⁺ 400 HPLC: 100%

### EXAMPLE 122

### Ethyl trans-4-[({4-[4-(4-acetylphenyl)phenyl]thien-2-yl}carboxamido)methyl]cyclohexanecarboxylate

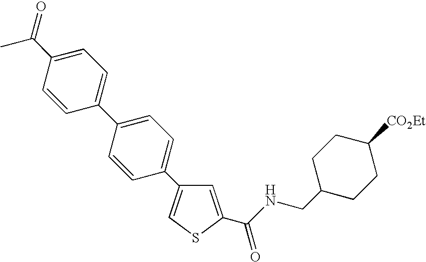

### Stage 1: Methyl 4-(4′-acetyl-1,1′-biphenyl-4-yl)thiophene-2-carboxylate

The product (1.88 g) is obtained according to the process of Example 1, using the compound of Example 71, Stage 1, and (4-acetylphenyl)boronic acid as substrates.

Yield: 83% ¹H NMR (DMSO) δ (ppm): 8.35 (s, 1H), 8.30 (s, 1H), 8.05 (m, 2H), 7.90 (m, 4H), 7.85 (m, 2H), 3.90 (s, 3H), 2.60 (s, 3H) HPLC: 100%

### Stage 2: 4-(4′-Acetyl-1,1′-biphenyl-4-yl)thiophene-2-carboxylic acid

The product (1.56 g) is obtained according to the process of Stage 7 of Example 18, using the compound obtained in the preceding Stage 1 as substrate.

Yield: 86% ¹H NMR (DMSO) δ (ppm): 13.25 (bs, 1H), 8.30 (s, 1H), 8.20 (s, 1H), 8.05 (m, 2H), 7.90 (m, 4H), 7.85 (m, 2H), 2.60 (s, 3H)

### Stage 3: Ethyl trans-4-[({4-[4-(4-acetylphenyl)phenyl]thien-2-yl}carboxamido)-methyl]cyclohexanecarboxylate

The product (221 mg) is obtained according to the process of Stage 1 of Example 80, using the compound obtained in the preceding Stage 2 and the product from Preparation 10 as substrates.

Yield: 73% ¹H NMR (DMSO) δ (ppm): 8.55 (bs, 1H), 8.30 (s, 1H), 8.15 (s, 1H), 8.05 (m, 2H), 7.90 (m, 6H), 4.05 (q, 2H), 3.10 (m, 2H), 2.60 (s, 3H), 2.25 (m, 1H), 2.95 (m, 2H), 2.80 (m, 2H), 1.55 (m, 1H), 1.30 (m, 2H), 1.15 (t, 3H), 1.00 (m, 2H) HPLC: 97%

### EXAMPLE 123

### trans-4-[({4-[4-(4-Acetylphenyl)phenyl]thien-2-yl}carboxamido)-methyl]cyclohexanecarboxylic acid

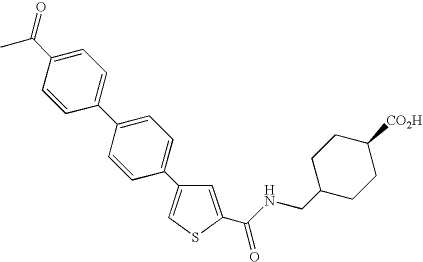

The product (100.4 mg) is obtained according to the process of Example 27, using the compound obtained in Example 122 as substrate.

Yield: 48% ¹H NMR (DMSO) δ (ppm): 12.00 (bs, 1H), 8.55 (bs, 1H), 8.35 (s, 1H), 8.15 (s, 1H), 8.05 (m, 2H), 7.85 (m, 6H), 3.15 (m, 2H), 2.65 (s, 3H), 2.15 (m, 1H), 1.95 (m, 2H), 1.85 (m, 2H), 1.50 (m, 1H), 1.25 (m, 2H), 1.00 (m, 2H) HPLC: 100%

### EXAMPLE 124

### Methyl trans-3-(3-{[4-(4-trifluoromethoxyphenyl)thien-2-yl]-carboxamido}cyclohexyl)propanoate

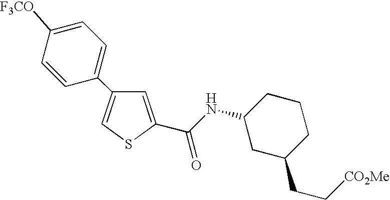

The product (52.7 mg) is obtained according to the process of Example 26, using the compounds from Preparation 5 and from Preparation 23 as substrates.

Yield: 15% ¹H NMR (CDCl₃) δ (ppm): 7.65 (s, 1H), 7.55 (m, 2H), 7.50 (s, 1H), 7.25 (m, 2H), 5.8 (dd, 1H), 4.0 (m, 1H), 3.65 (s, 3H), 2.4 (m, 2H), 2.15 (m, 2H), 1.8 (m, 2H), 1.5 (m, 2H), 1.45 (m, 2H), 1.15 (m, 2H), 0.9 (m, 2H) MS: MH⁺ 478 (+Na) HPLC: 100%

### EXAMPLE 125

### trans-3-(3-{[4-(4-Trifluoromethoxyphenyl)thien-2-yl]-carboxamido}cyclohexyl)propanoic acid

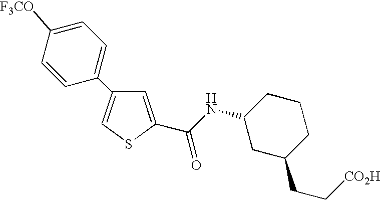

The product (24.4 mg) is obtained according to the process of Example 27, using the compound obtained in Example 124 as substrate.

Yield: 48% ¹H NMR (DMSO) δ (ppm): 12.0 (m, 1H), 8.35 (m, 2H), 8.35 (bs, 1H), 8.25 (m, 2H), 8.1 (s, 1H), 7.80 (m, 2H), 7.5 (m, 2H), 3.8 (m, 1H), 2.25 (m, 2H), 1.9 (m, 2H), 1.7 (m, 2H), 1.5 (m, 2H), 1.30 (m, 3H), 0.9 (m, 2H) MS: MH⁻ 440 HPLC: 100%

### EXAMPLE 126

### Methyl trans-(3-{[4-(4-trifluoromethoxyphenyl)thien-2-yl]-carboxamido}cyclohexyl)acetate

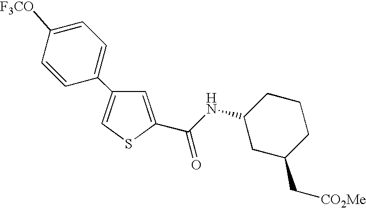

The product (188 mg) is obtained according to the process of Stage 1 of Example 80, using the compound obtained in Stage 2 of Preparation 5 and the compound from Preparation 24 as substrates.

Yield: 39% ¹H NMR (CDCl₃) δ (ppm): 7.7 (s, 1H), 7.6 (m, 2H), 7.5 (s, 1H), 5.80 (d, 1H), 4.0 (m, 1H), 3.6 (s, 3H), 2.3 (m, 2H), 2.1 (m, 2H), 2.0 (m, 1H), 1.80 (m, 2H), 1.5 (m, 1H), 1.3 (m, 1H), 1.0(m, 2H) MS: MH⁺ 442 HPLC: 100%

### EXAMPLE 127

### trans-(3-{[4-(4-Trifluoromethoxyphenyl)thien-2-yl]-carboxamido}cyclohexyl)acetic acid

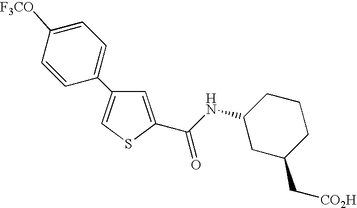

The product (0.161 g) is obtained according to the process of Example 27, using the compound obtained in Example 126 as substrate.

Yield: 89% ¹H NMR (CDCl₃) δ (ppm): 12.30 (m, 1H), 8.65 (m, 2H), 8.35 (bs, 1H), 8.15 (s, 1H), 7.75 (m, 2H), 3.75 (m, 1H), 2.1 (m, 2H), 1.85 (m, 2H), 1.75 (m, 3H), 1.45 (m, 1H), 1.40 (m, 3H), 1.1 (m, 1H), 0.9 (m, 1H) MS: MH⁺ 450 (+Na), MH⁻ 426 HPLC: 99.3%

### EXAMPLE 128

### Methyl trans-3-(4-{[4-(4-trifluoromethoxyphenyl)thien-2-yl]-carboxamido}cyclohexyl)propanoate

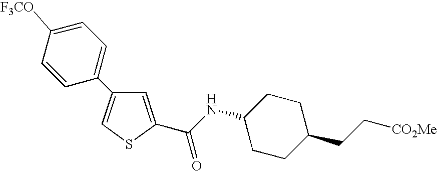

The product (143 mg) is obtained according to the process of Stage 1 of Example 80, using the compound obtained in Stage 2 of Preparation 5 and the compound from Preparation 25 as substrates.

Yield: 30% ¹H NMR (CDCl₃) δ (ppm): 7.7 (s, 1H), 7.6 (m, 2H), 7.5 (s, 1H), 7.25 (m, 2H), 5.7 (d, 1H), 4.0 (m, 1H), 3.7 (s, 3H), 2.3 (m, 2H), 2.20 (m, 2H), 1.80 (m, 2H), 1.5 (m, 1H), 1.45 (m, 2H), 1.30 (m, 1H), 1.0 (m, 4H) MS: MH⁺ 456 HPLC: 100%

### EXAMPLE 129

### trans-3-(4-{[4-(4-Trifluoromethoxyphenyl)thien-2-yl]-carboxamido}cyclohexyl)propanoic acid

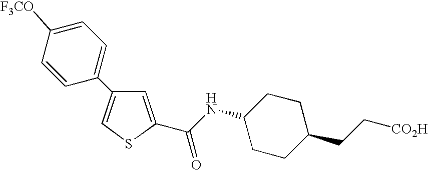

The product (46 mg) is obtained according to the process of Example 27, using the compound obtained in Example 128 as substrate.

Yield: 25% ¹H NMR (DMSO) δ (ppm): 12.30 (m, 1H), 8.35 (m, 2H), 8.20 (s, 1H), 7.75 (m, 2H), 7.50 (m, 2H), 3.75 (m, 1H), 2.1 (m, 2H), 1.85 (m, 2H), 1.75 (m, 2H), 1.45 (m, 2H), 1.30 (m, 3H), 1.1 (m, 2H) MS: MH⁻ 440 HPLC: 99.0%

### EXAMPLE 130

### Methyl [4-({[4-(4-trifluoromethoxyphenyl)thien-2-yl]carboxamido}-methyl)cyclohexyl]acetate

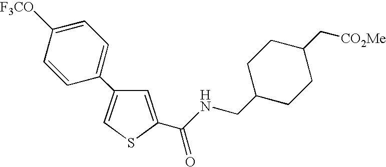

The product (189 mg) is obtained according to the process of Stage 1 of Example 80, using the compound obtained in Stage 2 of Preparation 5 and the compound from Preparation 26 as substrates.

Yield: 39% ¹H NMR (CDCl₃) δ (ppm): 7.7 (s, 1H), 7.6 (m, 2H), 7.5 (s, 1H), 7.20 (m, 2H), 6.0 (m, 1H), 3.7 (s, 3H), 3.4 (m, 2H), 2.3 (m, 2H), 2.0 (m, 1H), 1.80 (m, 2H), 1.5 (m, 2H), 1.45 (m, 3H), 1.0 (m, 2H) MS: MH⁺ 456 HPLC: 96.8%

### EXAMPLE 131

### [4-({[4-(4-Trifluoromethoxyphenyl)thien-2-yl]carboxamido}methyl)-cyclohexyl]acetic acid

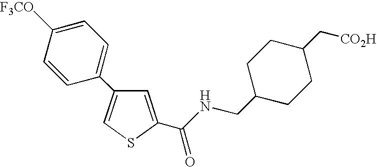

The product (104 mg) is obtained according to the process of Example 27, using the compound obtained in Example 130 as substrate.

Yield: 26% ¹H NMR (DMSO) δ (ppm): 12.30 (m, 1H), 8.50 (bs, 2H), 8.25 (s, 1H), 8.10 (s, 1H), 7.85 (m, 2H), 7.50 (m, 2H), 3.15 (m, 2H), 2.15 (m, 2H), 1.85 (m, 1H), 1.70 (m, 2H), 1.50 (m, 3H), 1.35 (m, 3H), 0.9 (m, 1H) MS: MH⁺ 442, MH⁻ 440 HPLC: 96.4%

### EXAMPLE 132

### Ethyl 2-hydroxy-5-({[4-(4-trifluoromethoxyphenyl)thien-2-yl]-carboxamido}methyl)benzoate

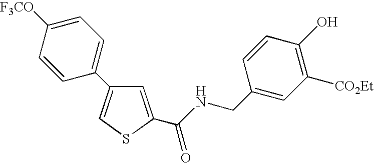

The product (202 mg) is obtained according to the process of Stage 1 of Example 80, using the compound obtained in Stage 2 of Preparation 5 and the compound from Preparation 27, to which 2.0 equivalents of triethylamine have been added beforehand, as substrates.

Yield: 39% ¹H NMR (DMSO) δ (ppm): 10.6 (s, 1H), 9.1 (bs, 1H), 8.3 (s, 1H), 8.20 (s, 1H), 7.80 (m, 3H), 7.6 (m, 1H), 7.50 (m, 2H), 7.0 (dd, 1H), 4.4 (m, 4H), 1.4 (t, 3H) MS: MH⁺ 466 HPLC: 96.2%

### EXAMPLE 133

### 2-Hydroxy-({[4-(4-trifluoromethoxyphenyl)thien-2-yl]-carboxamido}methyl)benzoic acid

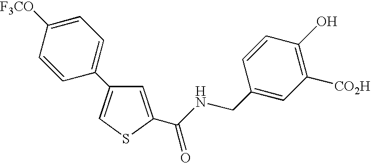

The product (176 mg) is obtained according to the process of Example 27, using the compound obtained in Example 132 as substrate.

Yield: 93% ¹H NMR (DMSO) δ (ppm): 14.0 (m, 1H), 11.40 (m, 1H), 9.10 (bs, 1H), 8.25 (s, 1H), 8.15 (s, 1H), 7.85 (m, 2H), 7.40 (m, 3H), 7.0 (m, 1H), 4.4 (m, 2H) MS: MH⁺ 438, MH⁻ 436 HPLC: 100%

### EXAMPLE 134

### Methyl 6-({[4-(4′-acetyl-4-biphenyl)thien-2-yl]carboxamido}-methyl)nicotinate

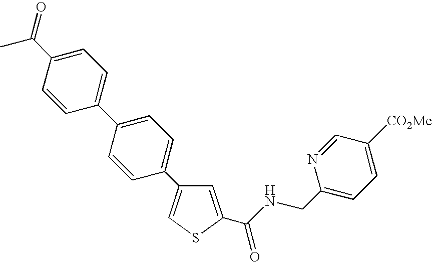

The product (66 mg) is obtained according to the process of Stage 2 of Preparation 1, using the product obtained in Stage 2 of Example 122 and the product from Preparation 28 as substrates and using 2 additional equivalents of triethylamine.

Yield: 45% MS: MH⁺ 471 HPLC: 94.2%

### EXAMPLE 135

### 6({[4-(4′-Acetyl-4-biphenyl)thien-2-yl]carboxamido}methyl)-nicotinic acid

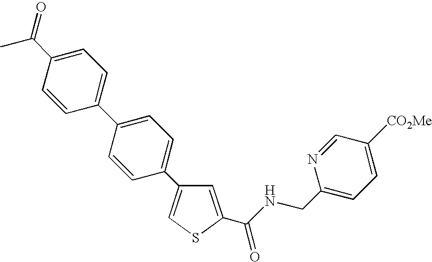

3.29 ml (11 equivalents) of 1.0.M sodium hydroxide are added to a solution of 0.155 g of the compound obtained in Example 134 in 2.0 ml of methanol. The reaction medium is stirred at 65° C. for 17 hours. The crystals formed are filtered off and washed under cold conditions successively with 10 ml of water, 10 ml of methanol and 10 ml of diethyl ether. The crystals are dried under reduced pressure at 50° C. in order to obtain 99.14 mg of the desired product.

Yield: 17% ¹H NMR (DMSO) δ (ppm): 9.30 (bs, 1H), 8.85 (s, 1H), 8.40 (s, 1H), 8.20 (s, 1H), 8.20 (s, 1H), 8.0 (m, 3H), 7.90 (m, 6H), 7.25 (dd, 1H), 4.6 (m, 2H), 2.6 (s, 3H) MS: MH⁺ 457 HPLC: 98.3%

### EXAMPLE 136

### N-(Pyridin-4-yl)-4-(4-trifluoromethoxyphenyl)thiophene-2-carboxamide

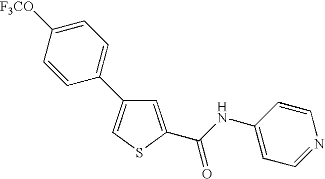

The product (0.158 g) is obtained according to the process of Example 26, using the compound from Preparation 5 and 4-aminopyridine as substrates.

Yield: 42% ¹H NMR (DMSO) δ (ppm): 10.60 (bs, 1H), 8.5 (m, 3H), 8.30 (s, 1H), 7.80 (m, 2H), 7.70 (m, 2H), 7.50 (m, 2H) MS: MH⁺ 365 HPLC: 99.6%

### EXAMPLE 137

### N-(2-Hydroxy-1-hydroxymethyl-1-methylethyl)-4-(4-trifluoromethoxyphenyl)thiophene-2-carboxamide

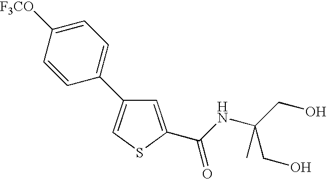

2-Amino-2-methylpropane-1,3-diol (1.1 equivalents), 1-(3-dimethylaminopropyl)-3-ethylcarbodiimide hydrochloride (1.1 equivalents), 1-hydroxybenzotriazole hydrate (1.1 equivalents) and, finally, N-ethyl-N,N-diisopropylamine (1.1 equivalents) are successively added to a solution of 200 mg of the compound obtained in Stage 2 of Preparation 5 in 3 ml of anhydrous dimethylformamide. The reaction medium is stirred overnight at ambient temperature and then treated conventionally in order to isolate the expected product.

Yield: 6% ¹H NMR (DMSO) δ (ppm): 8.30 (s, 1H), 8.12 (s, 1H), 7.84 (d, 2H), 7.46 (m, 3H), 4.78 (m, 2H), 3.61 (m, 4H), 1.28 (s, 3H) MS: MH⁺ 376 HPLC: 95.1%

### EXAMPLE 138

### N-(2-Hydroxypropyl)-4-(4-trifluoromethoxyphenyl)thiophene-2-carboxamide

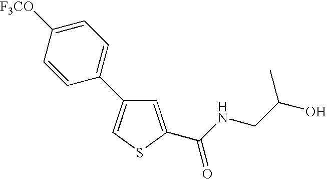

The product is obtained according to the process of Stage 3 of Example 6, using the compound obtained in Stage 2 of Preparation 5 and 1-aminopropan-2-ol as substrates.

Yield: 50% ¹H NMR (DMSO) δ (ppm): 8.49 (t, 1H), 8.29 (s, 1H), 8.13 (s, 1H), 7.81 (d, 2H), 7.46 (d, 2H), 4.77 (d, 1H), 3.78 (m, 1H), 3.19 (m, 2H), 1.08 (d, 3H) MS: MH⁺ 346 HPLC: 99.3%

### EXAMPLE 139

### N-(2-Hydroxy-1,1-dimethylethyl)-4-(4-trifluoromethoxyphenyl)-thiophene-2-carboxamide

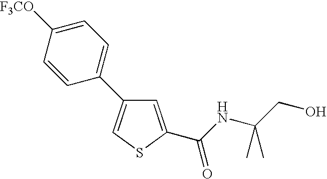

The product is obtained according to the process of Stage 3 of Example 6, using the compound obtained in Stage 2 of Preparation 5 and 2-amino-2-methylpropan-1-ol as substrates.

Yield: 80% ¹H NMR (DMSO) δ (ppm): 8.30 (s, 1H), 8.10 (s, 1H), 7.83 (d, 2H), 7.62 (s, 1H), 7.45 (d, 2H), 4.87 (t, 1H), 3.52 (d, 2H), 1.32 (s, 6H) MS: MH⁺ 360 HPLC: 99.3%

### EXAMPLE 140

### 4-(4-Trifluoromethoxyphenyl)-N-(tetrahydrofuran-2-ylmethyl)-thiophene-2-carboxamide

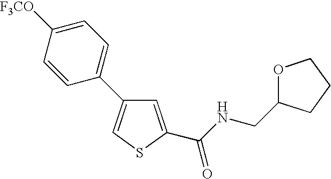

The product is obtained according to the process of Example 121, using 2-(aminomethyl)tetrahydrofuran as primary amine.

Yield: 62% ¹H NMR (DMSO) δ (ppm): 8.6 (t, 1H), 8.3 (s, 1H), 8.15 (s, 1H), 7.8 (d, 2H), 7.45 (d, 2H), 3.97 (m, 1H), 3.8 (m, 1H), 3.65 (m, 1H), 1.92 (m, 1H), 1.82 (m, 2H), 1.6 (m, 1H) MS: MH⁺ 372 HPLC: 97%

### EXAMPLE 141

### N-(2,2-Dimethyl-1,3-dioxolan-4-ylmethyl)-4-(4-trifluoromethoxyphenyl)thiophene-2-carboxamide

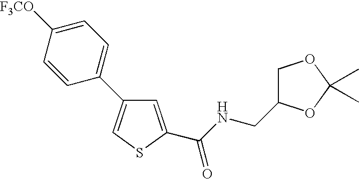

The product is obtained according to the process of Example 121, using (2,2-dimethyl-1,3-dioxolan-4-yl)methylamine as primary amine.

Yield: 34% ¹H NMR (CDCl₃) δ (ppm): 7.76 (s, 1H), 7.57 (m, 3H), 7.26 (d, 2H), 6.44 (bs, 1H), 4.35 (bs, 1H), 4.10 (t, 1H), 3.73 (m, 2H), 3.51 (m, 1H), 1.48 (s, 3H), 1.38 (s, 3H) MS: MH⁺ 402 HPLC: 98.6%

### EXAMPLE 142

### Ethyl 3-[4-(4-trifluoromethoxyphenyl)thiophene-2-carboxamido]-butyrate

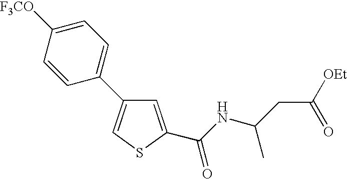

The product (355 mg) is obtained according to the process of Stage 3 of Example 6, using the compound obtained in Stage 2 of Preparation 5 and ethyl 3-aminobutyrate as substrates.

Yield: 85% ¹H NMR (DMSO) δ (ppm): 8.40 (d, 1H), 8.20 (s, 1H), 8.10 (s, 1H), 7.8 (d, 2H), 7.45 (d, 2H), 4.35 (m, 1H), 4.05 (q, 2H), 2.6 (dd, 1H), 2.5 (dd, 1H), 1.2 (d, 3H), 1.1 (t, 3H)

### EXAMPLE 143

### 3-[4-(4-Trifluoromethoxyphenyl)thiophene-2-carboxamido]butyric acid

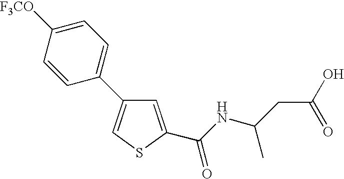

5 equivalents of lithium hydroxide are added to a solution of 355 mg of the compound of Example 142 in a solution (2/2/1: ethanol/water/DMF). After stirring for 17 hours at ambient temperature, the crude reaction mixture is concentrated under reduced pressure to remove the ethanol and the dimethylformamide. The aqueous phase is acidified with a 1N aqueous hydrochloric acid solution. The precipitate formed is filtered off, rinsed with water and then with diethyl ether, and finally dried overnight under reduced pressure at 50° C. (124 mg).

Yield: 38% ¹H NMR (DMSO) δ (ppm): 12.23 (bs, 1H), 8.39 (d, 1H), 8.22 (s, 1H), 8.13 (s, 1H), 7.81 (d, 2H), 7.46 (d, 2H), 4.30 (m, 1H), 2.56 (dd, 1H), 2.42 (dd, 1H), 1.20 (d, 3H) MS: MH⁺ 374 HPLC: 95.15%

### EXAMPLE 144

### N-[3-(Methylsulphinyl)propyl]-4-(4-trifluoromethoxyphenyl)-thiophene-2-carboxamide

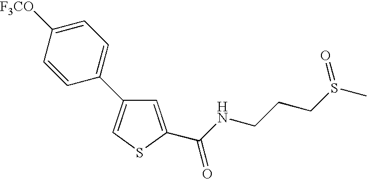

### Stage 1: N-[3-(Methylthio)propyl]-4-(4-trifluoromethoxyphenyl)thiophene-2-carboxamide

The product is obtained according to the process of Stage 3 of Example 6, using the compound obtained in Stage 2 of Preparation 5 and 3-(methylthio)propylamine as substrates.

Yield: 63%

### Stage 2: N-[3-(Methylsulphinyl)propyl]-4-(4-trifluoromethoxyphenyl)thiophene-2-carboxamide

82 mg (0.5 equivalent) of oxone and 87 mg (3.9 equivalents) of sodium hydrogencarbonate are added at 0° C. to a solution of 100 mg of the compound obtained in the preceding Stage 1 in 5 ml of a methanol/water (4/1) mixture. After stirring at ambient temperature for 4 hours, the crude reaction mixture is concentrated under reduced pressure to remove the methanol. The aqueous phase is subsequently extracted with ethyl acetate (2×10 ml). The combined organic phases are washed with a saturated sodium chloride solution, dried over magnesium sulphate and concentrated under reduced pressure. Chromatography of the residue on silica gel (dichloromethane/methanol: 98/5) makes it possible to obtain 40 mg of the expected product.

Yield: 40% ¹H NMR (CDCl₃) δ (ppm): 7.9 (s, 1H), 7.79 (t, 1H), 7.55 (m, 3H), 7.21 (d, 2H), 3.6 (m, 2H), 2.85 (m, 2H), 2.6 (s, 3H), 2.17 (m, 2H) MS: MH⁺ 392 HPLC: 99.6%

### EXAMPLE 145

### N-(2-Morpholin-4-ylethyl)-4-[4-(2,2,2-trifluoroethoxy)phenyl]-thiophene-2-carboxamide

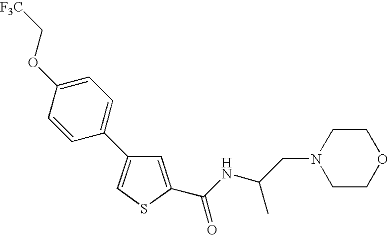

The product (45.4 mg) is obtained according to the process of Example 1, using the compound obtained in Stage 2 of Preparation 1 and the compound obtained in Stage 3 of Preparation 29 as substrates.

Yield: 54% ¹H NMR (CDCl₃) δ (ppm): 7.72 (s, 1H), 7.55 (d, 2H), 7.46 (s, 1H), 7.02 (d, 2H), 6.63 (s, 1H), 4.39 (m, 2H), 3.73 (m, 4H), 3.54 (m, 2H), 2.6 (m, 2H), 2.5 (m, 4H) MS: MH⁺ 416 HPLC: 97.8%

### EXAMPLE 146

### {4′-[5-(2-Morpholin-4-ylethylcarbamoyl)thiophen-3-yl]biphenyl-4-yl}-acetic acid, sodium salt

### Stage 1: Methyl {4′-[5-(2-morpholin-4-ylethylcarbamoyl)thiophen-3-yl]biphenyl-4-yl}acetate

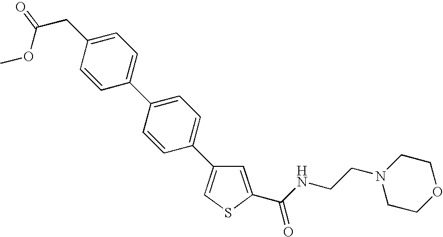

The product (118 mg) is obtained according to the process of Example 1, using the compound obtained in Stage 3 of Example 71 and the compound obtained in Stage 3 of Preparation 30 as substrates.

Yield: 31% ¹H NMR (CDCl₃) δ (ppm): 7.81 (s, 1H), 7.69 (m, 4H), 7.60 (m, 3H), 7.38 (m, 2H), 6.64 (s, 1H), 3.77 (m, 7H), 3.69 (m, 2H), 3.55 (m, 2H), 2.62 (m, 2H), 2.51 (m, 4H) MS: MH⁺ 465

### Stage 2: [4′-[5-(2-Morpholin-4-ylethylcarbamoyl)thiophen-3-yl]biphenyl-4-yl}acetic acid, sodium salt

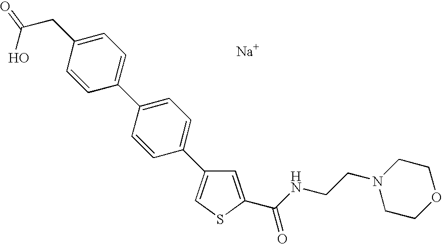

1.2 ml of water and 30.5 mg of lithium hydroxide (5 equivalents) are added to a solution of 118 mg of the compound obtained in the preceding Stage 1 in 1.5 ml of methanol. The reaction medium is stirred at 50° C. for 2 hours and then concentrated under reduced pressure. The residue obtained is taken up in ether and the insoluble material is removed. The ethereal solution, after several treatments with acid and base, results in the formation of a precipitate, which is isolated. The latter is taken up in ether, acetonitrile and methanol and then dried in an oven overnight, thus making it possible to obtain 73 mg of the desired product in sodium salt form of very low solubility.

Yield: 64% ¹H NMR (DMSO) δ (ppm): 8.56 (s, 1H), 8.20 (s, 1H), 8.04 (s, 1H), 7.7 (q, 4H), 7.52 (d, 2H), 7.27 (d, 2H), 3.34 (m, 4H), 3.23 (m, 2H), 2.50 (m, 4H), 2.40 (m, 4H) HPLC: 96% MS: MH⁺ 451

### EXAMPLE 147

### Ethyl trans-4-[({4-[4-(trifluoromethoxy)phenyl]thien-2-yl}-carbothioamido)methyl]cyclohexanecarboxylate

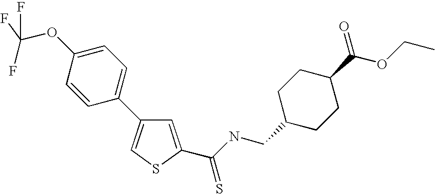

3.20 g (2 equivalents) of Lawesson's reagent are added to a solution of 1.692 g of the product obtained in Example 34 in 39 ml of anhydrous toluene. The reaction medium is brought to reflux for 24 h. After returning to ambient temperature, the crude reaction mixture is hydrolysed with a 5% hydrochloric acid solution and extracted with ethyl acetate. The organic phases are combined, dried over sodium sulphate and concentrated under vacuum to yield to a yellow oil, which is chromatographed on silica gel (eluant: cyclohexane/ethyl acetate 60:40) to yield to the desired product (543 mg).

Yield: 29% HPLC: 74%

### EXAMPLE 148

### trans-4-[({4-[4-(Trifluoromethoxy)phenyl]thien-2-yl}-carbothioamido)methyl]cyclohexanecarboxylic acid

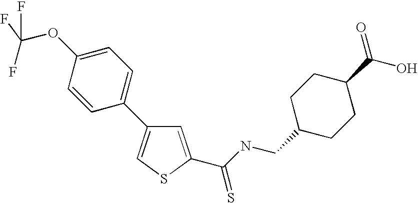

The product (85 mg) is obtained according to the process of Stage 7 of Example 18, using 0.124 g of the compound obtained in Example 147 as substrate.

Yield: 73% ¹H NMR (DMSO) δ (ppm): 12.2 (bs, 1H), 10.25 (s, 1H), 8.17 (s, 2H), 7.81 (dd, 2H), 7.45 (dd, 2H), 3.57 (t, 2H), 2.15 (m, 1H), 1.92 (m, 2H), 1.82 (m, 3H), 1.28 (m, 2H), 1.05 (m, 2H) MS: MH⁺ 444, MH⁻ 442 HPLC: 100%

### EXAMPLE 149

### Ethyl (2S)-2-{[4-(4-trifluoromethoxyphenyl)thien-2-yl]carboxamido}-3-methylbutanoate

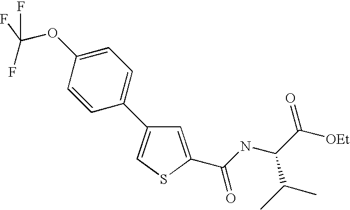

The product (389 mg) is obtained according to the process of Stage 3 of Example 6, using the compound obtained in Stage 2 of Preparation 5 and the hydrochloride of the methyl ester of L-valine as substrates.

Yield: 93% HPLC: 89%

### EXAMPLE 150

### (2S)-2-{[4-(4-Trifluoromethoxyphenyl)thien-2-yl]carboxamido}-3-methylbutanoic acid

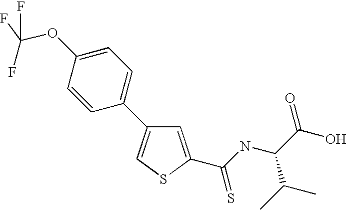

The product (120 mg) is obtained according to the process of Example 27, using the compound obtained in Example 149 as substrate.

Yield: 33% ¹H NMR (DMSO) δ (ppm): 13.20 (b, 1H), 8.55 (s, 1H), 8.50 (d, 1H), 8.20 (s, 1H), 7.85 (d, 2H), 7.45 (d, 2H), 4.30 (t, 1H), 2.20 (m, 1H), 0.90 (m, 6H) HPLC: 100%

### EXAMPLE 151

### N-(2-Morpholin-4-ylethyl)-4-(5-phenylpyridin-2-yl)thiophene-2-carboxamide

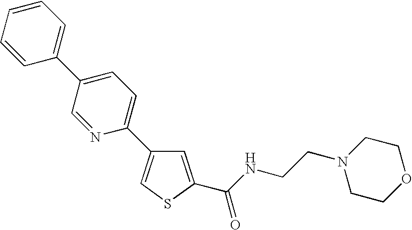

The product (74.8 mg) is obtained according to the process of Example 20, using the compound obtained in Preparation 2 and the compound obtained in Preparation 31 as substrates.

Yield: 44% ¹H NMR (DMSO) δ (ppm): 8.95 (m, 1H), 8.65 (bs, 1H), 8.50 (s, 1H), 8.40 (s, 1H), 8.20 (m, 1H), 7.95 (m, 1H), 7.80 (m, 2H), 7.55 (m, 2H), 7.45 (m, 1H), 3.60 (m, 4H), 3.40 (m, 2H), 2.50 (m, 2H), 2.40 (m, 4H) HPLC: 100% MS: MH⁺ 394

### EXAMPLE 152

### Pharmacological Studies on the Compounds of the Invention

### In Vitro Evaluation of the Inhibitory Activity of the Compounds of the Invention with Regard to MMP-12

The inhibitory activity of the compounds of formula (I) with regard to metalloproteinase-12 is evaluated by testing the ability of the compounds of the invention to inhibit the proteolysis of a peptide substrate by MMP-12.

The peptide substrate used (fluorogenic peptide-1: FP-1) in the test exhibits the following sequence: Mca-Pro-Leu-Gly-Leu-Dap(Dnp)-Ala-Arg-NH₂.

The inhibitory activity of a compound of formula (I) is expressed as IC₅₀ value, which represents the concentration of inhibitor at which a degree of inhibition of the metalloproteinase of 50% is observed.

The reaction begins with the sequential addition of 41 μl of substrate FP-1 (final concentration of 10 μM) to a buffer solution of 50 mM of Tris-HCl and 10 mM of CaCl₂, which comprises 5 mM of hydroxamic acid and 5 μl of the enzyme diluted in a 0.005% Brij-35 buffer solution. The microplates are incubated for 20 minutes at ambient temperature. The compounds of the invention are tested at concentrations varying from 0.3 to 30 μM.

The measurement of the amount of proteolysis of the peptide substrate is monitored by a measurement of absorbance at 405 nm using a microplate spectrophotometer, at ambient temperature. The IC₅₀ values are calculated from curves in which the percentage of the catalytic activity relative to the control is represented on the X axis and the concentration of inhibitor is represented on the Y axis.

The test described above for the inhibition of MMP-12 is adapted and used to determine the ability of the compounds of formula (I) to inhibit the metalloproteinases MMP-1, MMP-2, MMP-3, MMP-7, MMP-9, MMP-13 and MMP-14. The results obtained show that the compounds of the invention generally have IC₅₀ values for MMP-12 which are from 5 to more than 100 times lower than the IC₅₀ values obtained for the same compound with the other metalloproteinases tested, thus proving their ability to selectively inhibit metalloproteinase-12 (MMP-12). More specifically, the compounds of the present invention generally show a selectivity with a factor of greater than 50 with respect to the metalloproteinases mentioned above, except towards MMP-13. Thus, the compounds of the present invention also show an inhibitory activity with regard to MMP-13, making it possible to also use the pharmaceutical compositions comprising one or more compounds of the invention in the treatment of pathologies related to activity of MMP-13. Mention may be made, among these pathologies, by way of indication and without implied limitation, of cancer, osetoporosis, osteoarthritis, arthritis, rheumatoid arthritis, atherosclerosis, multiple sclerosis, cardiac insufficiency, asthma and chronic obstructive pulmonary disease.

A few activity results for the compounds of the invention with respect to several MMPs are presented in the table below, by way of example and without limititation of the invention.

| | IC₅₀ (μM) | IC₅₀ (μM) |
|---|---|---|
| Example | MMP-12 | MMP-13 |
| 2 | 0.35 | 30 |
| 9 | 0.105 | 1.5 |
| 11 | 0.91 | 4.8 |
| 14 | 0.86 | 2.075 |
| 21 | 0.46 | 11 |
| 22 | 0.915 | 11.6 |
| 27 | 0.845 | 6.1 |
| 35 | 0.405 | 3.45 |
| 37 | 0.427 | 4.53 |
| 41 | 0.883 | 5.167 |
| 43 | 0.715 | 5.367 |
| 45 | 0.92 | 2.85 |
| 47 | 0.436 | 3.59 |
| 55 | 0.615 | 15.75 |
| 63 | 0.49 | 3.8 |
| 65 | 0.685 | 2.9 |
| 70 | 0.022 | 0.285 |
| 72 | 0.057 | 0.13 |
| 73 | 0.355 | 0.4 |
| 74 | 0.465 | 6.245 |
| 75 | 0.34 | 0.27 |
| 76 | 0.48 | 3.7 |
| 81 | 0.5 | 3.7 |
| 84 | 0.56 | 3.06 |
| 99 | 0.45 | 5 |
| 101 | 0.99 | 3.7 |
| 103 | 0.05 | Nt |
| 105 | 0.05 | Nt |
| 107 | 0.05 | Nt |
| 109 | 0.165 | 0.4 |
| 111 | 0.7 | 2.2 |
| 113 | 0.14 | Nt |
| 115 | 0.25 | 3.32 |
| 117 | 0.16 | 0.31 |
| 119 | 0.13 | 0.78 |
| 120 | 0.66 | 6.1 |
| 121 | 0.72 | Nt |
| 123 | 0.018 | 0.031 |
| 125 | 0.32 | 3.95 |
| 129 | 0.78 | 6.20 |
| 131 | 0.425 | 3.3 |
| 133 | 0.95 | 2.65 |
| 135 | 0.042 | 0.08 |
| 150 | 0.78 | 10.05 |

* Nt: not tested

1- Compounds of formula (I): 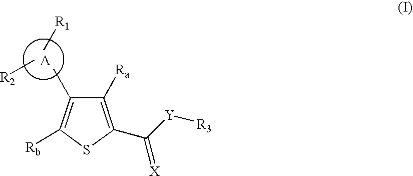
in which:
X represents an oxygen atom or a sulphur atom,
Y represents an oxygen atom, an —NH— group or an —N(C₁-C₆)alkyl- group,
Rₐ represents a group selected from hydrogen, halogen, (C₁-C₃)alkyl, hydroxyl and (C₁-C₃)alkoxy,
R_{b} represents a group selected from hydrogen, halogen and (C₁-C₃)alkyl,
A represents a group selected from phenyl, pyridyl, (C₅-C₆)cycloalkyl and (C₅-C₆)cycloalkenyl,
R₁ and R₂, which are identical or different, each represent, independently of each other, a group selected from:
hydrogen, halogen, cyano, nitro, halo(C₁-C₆)alkyl, halo(C₁-C₆)alkoxy, (C₁-C₆)alkyl, (C₂-C₆)alkenyl, (C₂-C₆)alkynyl,
—OR₄, —NR₄R₅, —S(O)R₄, —C(O)R₄, —CO₂R₄, —O—C(O)R₄, —C(O)NR₄R₅, —NR₅—C(O)R₄, —NR₅—SO₂R₄, -T-CN, -T-OR₄, -T-OCF₃, -T-NR₄R₅, -T-S(O)ₙR₄, -T- C(O)R₄, -T-CO₂R₄, -T-O—C(O)R₄, -T-C(O)NR₄R₅, -T-NR₅—C(O)R₄, -T-NR₅—SO₂R₄, —R₆ and -T-R₆, in which: [symbol]
n represents an integer from 0 to 2 inclusive, [symbol]
T represents a linear or branched (C₁-C₆)alkylene chain optionally substituted by one group selected from oxo, halogen, (C₁-C₆)alkoxy, hydroxyl, amino, mono(C₁-C₆)alkylamino, di(C₁-C₆)alkylamino and/or in which optionally one of the carbon atoms is replaced by an oxygen atom, a sulphur atom, an —NH— group or an —N(C₁-C₆)alkyl- group (it being understood that, in the case where one of the carbon atoms is replaced by a group as defined above, then the said alkylene chain comprises at least one sequence of two atoms) [symbol]
R₄ represents a hydrogen atom, a (C₁-C₆)alkyl, aryl, cycloalkyl group or a heterocycle, [symbol]
R₅ represents a hydrogen atom or a (C₁-C₆)alkyl group, [symbol]
R₆ represents a group selected from aryl, cycloalkyl and a heterocycle, each of these groups optionally being substituted from one to five identical or different groups selected, independently of each other, from halogen, cyano, nitro, oxo, halo(C₁-C₆)alkyl, halo(C₁-C₆)alkoxy, (C₁-C₆)alkyl, (C₁-C₆)alkenyl, —OR₄₀, —N_{R}₄₀R₅₀, —S(O)ₙ₁R₄₀, —C(O)R₄₀, —CO₂R₄₀, —O—C(O)R₄₀, —C(O)NR₄₀R₅₀, —NR₅₀—C(O)R₄₀, —NR₅₀—SO₂R₄₀, -T₁-CN, -T₁-OR₄₀, -T₁-OCF₃, -T₁-NR₄₀R₅₀, -T₁-S(O)ₙR₄₀, -T₁-C(O)R₄₀, -T₁-CO₂R₄₀, -T₁-O—C(O)R₄₀, -T₁-C(O)NR₄₀ R₅₀, -T₁-NR₅₀—C(O)R₄₀, -T₁-NR₅₀—SO₂R₄₀, -G₁ and -T₁-G₁, in which: [symbol]
R₄₀, R₅₀, T₁ and n₁ respectively have the same meanings as R₄, R₅, T and n as defined above, [symbol]
G₁ represents a group selected from aryl, cycloalkyl and a heterocycle, each of these groups optionally being substituted by 1 to 5 identical or different groups selected, independently of each other, from halogen, cyano, nitro, halo(C₁-C₆)alkyl, halo(C₁-C₆)alkoxy, (C₁-C₆)alkyl, hydroxyl, (C₁-C₆)alkoxy, phenoxy, benzyloxy, amino, mono(C₁-C₆)alkylamino, di(C₁-C₆)alkylamino, mercapto, (C₁-C₆)alkylthio, (C₁-C₇)acyl, (C₁-C₆)alkylsulphinyl, carboxyl, (C₁-C₆)alkoxycarbonyl, phenyl and a heterocycle,
R₃ represents an —R₇ or —U—R₁₁ group, in which:
R₇ represents a group selected from hydrogen, (C₁-C₆)alkyl, aryl, cycloalkyl and heterocycle, each cyclic system optionally being substituted by one to five identical or different groups selected, independently of each other, from halogen, cyano, nitro, halo(C₁-C₆)alkyl, halo(C₁-C₆)alkoxy, (C₁-C₆)alkyl, —OR₈, —NR₈R₉, —S(O)ₘR₈, —C(O)R₈, —CO₂R₈, —O—C(O)R₈, —C(O)NR₈R₉, —NR₉—C(O)R₈, —NR₉—SO₂R₈, —V—CN, —V—OR₈, —V—NR₈R₉, —V—S(O)ₘR₈, —V—C(O)R₈, —V—CO₂R₈, —V—O—C(O)R₈, —V—C(O)NR₈R₉, —V—NR₉—C(O)R₈, —V—NR₉—SO₂R₈, —R₁₀ and —V—R₁₀, in which: [symbol]
m represents an integer from 0 to 2 inclusive, [symbol]
V represents a group selected from a linear or branched (C₁-C₆)alkylene chain, a linear or branched (C₂-C₆)alkenylene chain, a cyclopropylene group and a linear or branched (C₂-C₆)alkylene chain in which one of the carbon atoms is replaced by an oxygen agom, a sulphur atom, an —NH— group or an —N(C₁-C₆)alkyl- group, [symbol]
R₈ represents a hydrogen atom, a (C₁-C₆)alkyl, aryl or cycloalkyl group or a heterocycle, [symbol]
R₉ represents a hydrogen atom or a (C₁-C₆)alkyl group, [symbol]
R₁₀ represents an aryl or cycloalkyl group or a heterocycle,
U represents a linear or branched (C₁-C₆)alkylene chain optionally substituted by one hydroxyl group or a linear or branched (C₂-C₆)alkylene chain in which one of the carbon atoms is replaced by an oxygen atom, a sulphur atom, an —NH— group or an —N(C₁-C₆)alkyl- group,
R₁₁ represents a group selected from halogen, —OR₁₂, —NR₁₂R₁₃, —S(O)ₚR₁₂, —C(O)R₁₂, —CO₂R₁₂, —O—C(O)R₁₂, —C(O)NR₁₂R₁₃, —NR₁₃—C(O)R₁₂, —NR₁₃—SO₂R₁₂ and —R₁₄, the latter group optionally being substituted by one to three identical or different groups selected, independently of each other, from halogen, cyano, nitro, halo(C₁-C₆)alkyl, halo(C₁-C₆)alkoxy, (C₁-C₆)alkyl, (C₂-C₆)alkenyl, (C₂-C₆)alkynyl, —OR₁₅, —NR₁₅R₁₆, —S(O)_{q}R₁₅, —C(O)R₁₅, —O—C(O)R₁₅, —CO₂R₁₅, —C(O)NR₁₅R₁₆, —NR₁₆—C(O)R₁₅, —NR₁₆—SO₂R₁₅, —R₁₇, —W—CN, —W—OR₁₅, —W—NR₁₅R₁₆, —W—S(O)_{q}R₁₅, —W—C(O)R₁₅, —W—CO₂R₁₅, —W—O—C(O)R₁₅, —W—C(O)NR₁₅R₁₆, —W—NR₁₆—C(O)R₁₅, —W—NR₁₆—SO₂R₁₅, —W—R₁₇ and —C(O)—W₁—CO₂R₁₅, in which: [symbol]
R₁₂ represents a hydrogen atom, a (C₁-C₆)alkyl, aryl, cycloalkyl group or a heterocycle, [symbol]
R₁₃ represents a hydrogen atom or a (C₁-C₆)alkyl group, [symbol]
R₁₄ represents an aryl or cycloalkyl group or a heterocycle, [symbol]
R₁₅ represents a hydrogen atom, a (C₁-C₆)alkyl, aryl or cycloalkyl group or a heterocycle, [symbol]
R₁₆ represents a hydrogen atom or a (C₁-C₆)alkyl group, [symbol]
R₁₇ represents an aryl or cycloalkyl group or a heterocycle, [symbol]
p represents an integer from 0 to 2 inclusive, [symbol]
q represents an integer from 0 to 2 inclusive, [symbol]
W represents a group selected from a linear or branched (C₁-C₆)alkylene chain, a linear or branched (C₂-C₆)alkenylene chain, a cyclopropylene group and a linear or branched (C₂-C₆)alkylene chain in which one of the carbon atoms is replaced by an oxygen atom, a sulphur atom, an —NH— group or an —N(C₁-C₆)alkyl- group, [symbol]
W₁ represents a linear or branched (C₁-C₆)alkylene chain,
their isomers, and their addition salts with a pharmaceutically acceptable acid or base, it being understood that:
the term “aryl group” denotes a monocyclic or bicyclic aromatic system comprising from 4 to 10 carbon atoms, it being understood that, in the case of a bicyclic system, one of the rings exhibits an aromatic nature and the other ring is aromatic or unsaturated,
the term “cycloalkyl group” denotes a saturated or partially unsaturated, fused or bridged, bicyclic or monocyclic system comprising from 3 to 12 carbon atoms,
the term “a heterocycle” denotes a saturated, unsaturated or aromatic, 3- to 12-membered, fused or bridged, bicyclic or monocyclic system comprising from 1 to 4 identical or different heteroatoms selected, independently of each other, from oxygen, sulphur and nitrogen and optionally comprising 1 or 2 oxo or thioxo groups, it being understood that, in the case of a bicyclic system, one of the rings exhibits an aromatic nature and the other ring is aromatic or unsaturated, or both rings are saturated, or one of the rings is saturated and the other ring is unsaturated, or both rings are unsaturated,
the term “(C₁-C₆)alkyl group” denotes a linear or branched carbonaceous chain comprising from 1 to 6 carbon atoms,
the term “halo(C₁-C₆)alkyl group” denotes a linear or branched carbonaceous chain comprising from 1 to 6 carbon atoms and substituted by 1 to 6 halogen atoms,
the term “halo(C₁-C₆)alkoxy group” denotes a linear or branched carbonaceous chain comprising from 1 to 6 carbon atoms and substituted by 1 to 6 halogen atoms, the said chain being connected to the compound of formula (I) by an oxygen atom,
the term “halogen atom” denotes an atom selected from bromine, chlorine, fluorine and iodine,
the term “acyl group” denotes a hydrogen atom, an alkyl group as defined above, a cycloalkyl comprising 3 to 6 carbon atoms or a phenyl group bonded through an oxo group to the compounds of formula (I),
it also being understood that the compounds of formula (I) are not:
ethyl 5-methyl-4-phenylthiophen-2-ylcarboxylate,
5-methyl-4-phenylthiophen-2-ylcarboxylic acid,
methyl 3-hydroxy-4-phenylthiophen-2-ylcarboxylate,
methyl 3-methoxy-4-phenylthiophen-2-ylcarboxylate,
3-methoxy-4-phenylthiophen-2-ylcarboxylic acid,
methyl 4-(4-methoxyphenyl)thiophen-2-ylcarboxylate,
methyl 4-phenylthiophen-2-ylcarboxylate,
4-(4-methoxyphenyl)thiophen-2-ylcarboxylic acid,
4-phenylthiophen-2-ylcarboxylic acid,
4-(4-tert-butylphenyl)thiophen-2-ylcarboxylic acid,
methyl 5-chloro-3-hydroxy-4-phenylthiophen-2-ylcarboxylate,
4-(3,5-dimethylphenyl)thiophen-2-ylcarboxylic acid,
methyl 4-[4-(acetylamino)phenyl]thiophen-2-ylcarboxylate,
ethyl 4-phenylthiophen-2-ylcarboxylate,
4-phenylthiophen-2-ylcarboxamide,
N-methyl-4-phenylthiophen-2-ylcarboxamide,
N,N-dimethyl-4-phenylthiophen-2-ylcarboxamide,
5-methyl-4-phenylthiophen-2-ylcarboxamide,
N-methyl-5-methyl-4-phenylthiophen-2-ylcarboxamide,
N,N-dimethyl-5-methyl-4-phenylthiophen-2-ylcarboxamide,
methyl 2-{[4-(4-methoxyphenyl)thiophen-2-yl]carboxamido}benzoate,
N-[3-(trifluoromethyl)phenyl]-4-(4-methoxyphenyl)thiophen-2-ylcarboxamide,
methyl 4-(2-bromophenyl)thiophen-2-ylcarboxylate,
methyl 4-(3-bromophenyl)thiophen-2-ylcarboxylate,
methyl 4-(4-bromophenyl)thiophen-2-ylcarboxylate,
N-(2-methoxyethyl)-4-(4-methoxyphenyl)thiophen-2-ylcarboxamide,
N-isopropyl-4-(4-methoxyphenyl)thiophen-2-ylcarboxamide,
4-(4-methoxyphenyl)-N-(2-morpholinoethyl)thiophen-2-ylcarboxamide,
4-(4-chlorophenyl)-N-(2-methoxyethyl)thiophen-2-ylcarboxamide,
4-(4-chlorophenyl)-N-(2-morpholinoethyl)thiophen-2-ylcarboxamide,
4-(4-chlorophenyl)-N-(2-phenylethyl)thiophen-2-ylcarboxamide,
4-(4-chlorophenyl)-N-(tetrahydrofurylmethyl)thiophen-2-ylcarboxamide,
and 4-(4-methoxyphenyl)-N-(tetrahydrofurylmethyl)thiophen-2-ylcarboxamide;
it also being understood that:
if Rₐ represents a hydrogen atom, A represents a cyclopenten-1-yl group substituted in the 2 position by an R₁ group taking the definition thienyl optionally substituted, then R_{b} represents a group selected from hydrogen, halogen and (C₂-C₃)alkyl,
if R₃ represents an R₇ group taking the definition heterocycle, then the said heterocycle cannot represent a 1-azabicyclo[2.2.2]oct-3-yl group,
and if R₃ represents an R₇ group taking the definition phenyl substituted in the para position by an R₁₀ group, then the said Rio group cannot represent a 5-methyl-4,5-dihydro-3-oxo-2H-pyridazin-6-yl group.

2- Compounds of formula (I) according to claim 1, in which:
X represents an oxygen atom or a sulphur atom,
Y represents an oxygen atom, an —NH— group or an —N(C₁-C₆)alkyl- group,
Rₐ represents a group selected from hydrogen, halogen, (C₁-C₃)alkyl, hydroxyl and (C₁-C₃)alkoxy,
R_{b} represents a group selected from hydrogen, halogen and (C₁-C₃)alkyl,
A represents a group selected from phenyl, (C₅-C₆)cycloalkyl and (C₅-C₆)cycloalkenyl,
R₁ and R₂, which are identical or different, each represent, independently of each other, a group selected from:
hydrogen, halogen, cyano, nitro, halo(C₁-C₆)alkyl, halo(C₁-C₆)alkoxy, (C₁-C₆)alkyl, (C₂-C₆)alkenyl, (C₂-C₆)alkynyl,
—OR₄, —NR₄R₅, —S(O)ₙR₄, —C(O)R₄, —CO₂R₄, —O—C(O)R₄, —C(O)NR₄R₅, —NR₅—C(O)R₄, —NR₅—SO₂R₄, -T-CN, -T-OR₄, -T-OCF₃, -T-NR₄R₅, -T-S(O)ₙR₄, -T- C(O)R₄, -T-CO₂R₄, -T-O—C(O)R₄, -T-C(O)NR₄R₅, -T-NR₅—C(O)R₄, -T-NR₅—SO₂R₄, —R₆ and -T-R₆, in which: [symbol]
n represents an integer from 0 to 2 inclusive, [symbol]
T represents a linear or branched (C₁-C₆)alkylene chain optionally substituted by a group selected from oxo, halogen, (C₁-C₆)alkoxy, hydroxyl, amino, mono(C₁-C₆)alkylamino and di(C₁-C₆)alkylamino and/or in which optionally one of the carbon atoms is replaced by an oxygen atom, a sulphur atom, an —NH— group or an —N(C₁-C₆)alkyl- group (it being understood that, in the case where one of the carbon atoms is replaced by a group as defined above, then the said alkylene chain comprises at least one sequence of two atoms) [symbol]
R₄ represents a hydrogen atom, a (C₁-C₆)alkyl, aryl or cycloalkyl group or a heterocycle, [symbol]
R₅ represents a hydrogen atom or a (C₁-C₆)alkyl group, [symbol]
R₆ represents a group selected from aryl, cycloalkyl and a heterocycle, each of these groups optionally being substituted by one to five identical or different groups selected, independently of each other, from halogen, cyano, nitro, oxo, halo(C₁-C₆)alkyl, halo(C₁-C₆)alkoxy, (C₁-C₆)alkyl, (C₁-C₆)alkenyl, —OR₄₀, —NR₄₀R₅₀, —S(O)ₙ₁R₄₀, —C(O)R₄₀, —CO₂R₄₀, —O—C(O)R₄₀, —C(O)NR₄₀R₅₀, —NR₅₀—C(O)R₄₀, —NR₅₀—SO₂R₄₀, -T₁-CN, -T₁-OR₄₀, -T₁-OCF₃, -T₁-NR₄₀R₅₀, -T₁-S(O)ₙR₄₀, -T₁-C(O)R₄₀, -T₁-CO₂R₄₀, -T₁-O—C(O)R₄₀, -T₁-C(O)NR₄₀R₅₀, -T₁-NR₅₀—C(O)R₄₀, -T₁-NR₅₀—SO₂R₄₀, —G₁ and -T₁-G₁, in which: [symbol]
R₄₀, R₅₀, T₁ and n₁ respectively have the same meanings as R₄, R₅, T and n as defined above, [symbol]
G₁ represents a group selected from aryl, cycloalkyl and a heterocycle, each of these groups optionally being substituted by 1 to 5 identical or different groups selected, independently of each other, from halogen, cyano, nitro, halo(C₁-C₆)alkyl, halo(C₁-C₆)alkoxy, (C₁-C₆)alkyl, hydroxyl, (C₁-C₆)alkoxy, phenoxy, benzyloxy, amino, mono(C₁-C₆)alkylamino, di(C₁-C₆)alkylamino, mercapto, (C₁-C₆)alkylthio, (C₁-C₇)acyl, (C₁-C₆)alkylsulphinyl, carboxyl, (C₁-C₆)alkoxycarbonyl, phenyl and a heterocycle,
R₃ represents an —R₇ or —U—R₁₁ group, in which:
R₇ represents a group selected from hydrogen, (C₁-C₆)alkyl, aryl, cycloalkyl and heterocycle, each cyclic system optionally being substituted by one to five identical or different groups selected, independently of each other, from halogen, cyano, nitro, hato(C₁-C₆)alkyl, halo(C₁-C₆)alkoxy, (C₁-C₆)alkyl, —OR₈, —NR₈R₉, —S(O)ₘR₈, —C(O)R₈, —CO₂R₈, —O—C(O)R₈, —C(O)NR₈R₉, —NR₉—C(O)R₈, —NR₉—SO₂R₈, —V—CN, —V—OR₈, —V—NR₈R₉, —VS(O)ₘR₈, —V—C(O)R₈, —V—CO₂R₈, —V—O—C(O)R₈, —V—C(O)NR₈R₉, —V—NR₉—C(O)R₈, —V—NR₉—SO₂R₈, —R₁₀ and —V—R₁₀, in which: [symbol]
m represents an integer from 0 to 2 inclusive, [symbol]
V represents a group selected from a linear or branched (C₁-C₆)alkylene chain, a linear or branched (C₂-C₆)alkenylene chain, a cyclopropylene group and a linear or branched (C₂-C₆)alkylene chain in which one of the carbon atoms is replaced by an oxygen agom, a sulphur atom, an —NH— group or an —N(C₁-C₆)alkyl- group, [symbol]
R₈ represents a hydrogen atom, a (C₁-C₆)alkyl, aryl or cycloalkyl group or a heterocycle, [symbol]
R₉ represents a hydrogen atom or a (C₁-C₆)alkyl group, [symbol]
R₁₀ represents an aryl or cycloalkyl group or a heterocycle,
U represents a linear or branched (C₁-C₆)alkylene chain optionally substituted by a hydroxyl group or a linear or branched (C₂-C₆)alkylene chain in which one of the carbon atoms is replaced by an oxygen atom, a sulphur atom, an —NH— group or an —N(C₁-C₆)alkyl- group,
R₁₁ represents a group selected from halogen, —OR₁₂, —NR₁₂R₁₃, —S(O)ₚR₁₂, —C(O)R₁₂, —CO₂R₁₂, —O—C(O)R₁₂, —C(O)NR₁₂R₁₃, —NR₁₃—C(O)R₁₂, —NR₁₃—SO₂R₁₂ and —R₁₄, the latter group optionally being substituted by one to three identical or different groups selected, independently of each other, from halogen, cyano, nitro, halo(C₁-C₆)alkyl, halo(C₁-C₆)alkoxy, (C₁-C₆)alkyl, (C₂-C₆)alkenyl, (C₂-C₆)alkynyl, —OR₁₅, —NR₁₅R₁₆, —S(O)_{q}R₁₅, —C(O)R₁₅, —O—C(O)R₁₅, —CO₂R₁₅, —C(O)NR₁₅R₁₆, —NR₁₆—C(O)R₁₅, —NR₁₆—SO₂R₁₅, —R₁₇, —W—CN, —W—OR,₅, —W—NR₁₅R₆, —W—S(O)_{q}R₁₅, —W—C(O)R₁₅, —W—CO₂R₁₅, —W—O—C(O)R₁₅, —W—C(O)NR₁₅R₁₆, —W—NR₁₆—C(O)R₁₅, W—NR₁₆—SO₂R₁₅, —W—R₁₇ and —C(O)—W₁—CO₂R₁₅, in which: [symbol]
R₁₂ represents a hydrogen atom, a (C₁-C₆)alkyl, aryl or cycloalkyl group or a heterocycle, [symbol]
R₁₃ represents a hydrogen atom or a (C₁-C₆)alkyl group, [symbol]
R₁₄ represents an aryl or cycloalkyl group or a heterocycle, [symbol]
R₁₅ represents a hydrogen atom, a (C₁-C₆)alkyl, aryl or cycloalkyl group or a heterocycle, [symbol]
R₁₆ represents a hydrogen atom or a (C₁-C₆)alkyl group, [symbol]
R₁₇ represents an aryl or cycloalkyl group or a heterocycle, [symbol]
p represents an integer from 0 to 2 inclusive, [symbol]
q represents an integer from 0 to 2 inclusive, [symbol]
W represents a group selected from a linear or branched (C₁-C₆)alkylene chain, a linear or branched (C₂-C₆)alkenylene chain, a cyclopropylene group and a linear or branched (C₂-C₆)alkylene chain in which one of the carbon atoms is replaced by an oxygen atom, a sulphur atom, an —NH— group or an —N(C₁-C₆)alkyl- group, [symbol]
W₁ represents a linear or branched (C₁-C₆)alkylene chain, their isomers, and their addition salts with a pharmaceutically acceptable acid or base,
it also being understood that the compounds of formula (I) are not:
ethyl 5-methyl-4-phenylthiophen-2-ylcarboxylate,
5-methyl-4-phenylthiophen-2-ylcarboxylic acid,
methyl 3-hydroxy-4-phenylthiophen-2-ylcarboxylate,
methyl 3-methoxy-4-phenylthiophen-2-ylcarboxylate,
3-methoxy-4-phenylthiophen-2-ylcarboxylic acid,
methyl 4-(4-methoxyphenyl)thiophen-2-ylcarboxylate,
methyl 4-phenylthiophen-2-ylcarboxylate,
4-(4-methoxyphenyl)thiophen-2-ylcarboxylic acid,
4-phenylthiophen-2-ylcarboxylic acid,
4-(4-tert-butylphenyl)thiophen-2-ylcarboxylic acid,
methyl 5-chloro-3-hydroxy-4-phenylthiophen-2-ylcarboxylate,
4-(3,5-dimethylphenyl)thiophen-2-ylcarboxylic acid,
methyl 4-[4-(acetylamino)phenyl]thiophen-2-ylcarboxylate,
ethyl 4-phenylthiophen-2-ylcarboxylate,
4-phenylthiophen-2-ylcarboxamide,
N-methyl-4-phenylthiophen-2-ylcarboxamide,
N,N-dimethyl-4-phenylthiophen-2-ylcarboxamide,
5-methyl-4-phenylthiophen-2-ylcarboxamide,
N-methyl-5-methyl-4-phenylthiophen-2-ylcarboxamide,
N,N-dimethyl-5-methyl-4-phenylthiophen-2-ylcarboxamide,
methyl 2-{[4-(4-methoxyphenyl)thiophen-2-yl]carboxamido}benzoate,
N-[3-(trifluoromethyl)phenyl]-4-(4-methoxyphenyl)thiophen-2-ylcarboxamide,
methyl 4-(2-bromophenyl)thiophen-2-ylcarboxylate,
methyl 4-(3-bromophenyl)thiophen-2-ylcarboxylate,
methyl 4-(4-bromophenyl)thiophen-2-ylcarboxylate,
N-(2-methoxyethyl)-4-(4-methoxyphenyl)thiophen-2-ylcarboxamide,
N-isopropyl-4-(4-methoxyphenyl)thiophen-2-ylcarboxamide,
4-(4-methoxyphenyl)-N-(2-morpholinoethyl)thiophen-2-ylcarboxamide,
4-(4-chlorophenyl)-N-(2-methoxyethyl)thiophen-2-ylcarboxamide,
4-(4-chlorophenyl)-N-(2-morpholinoethyl)thiophen-2-ylcarboxamide,
4-(4-chlorophenyl)-N-(2-phenylethyl)thiophen-2-ylcarboxamide,
4-(4-chlorophenyl)-N-(tetrahydrofurylmethyl)thiophen-2-ylcarboxamide,
and 4-(4-methoxyphenyl)-N-(tetrahydrofurylmethyl)thiophen-2-ylcarboxamide;
it also being understood that:
if Rₐ represents a hydrogen atom, A represents a cyclopenten-1-yl group substituted in the 2 position by an R₁ group taking the definition thienyl optionally substituted, then R_{b} represents a group selected from hydrogen, halogen and (C₂-C₃)alkyl,
if R₃ represents an R₇ group taking the definition heterocycle, then the said heterocycle cannot represent a 1-azabicyclo[2.2.2]oct-3-yl group,
and if R₃ represents an R₇ group taking the definition phenyl substituted in the para position by an R₁₀ group, then the said R₁₀ group cannot represent a 5-methyl-4,5-dihydro-3-oxo-2H-pyridazin-6-yl group.

3- Compounds of formula (I) according to claim 1, characterized in that they represent compounds of formula (IA): 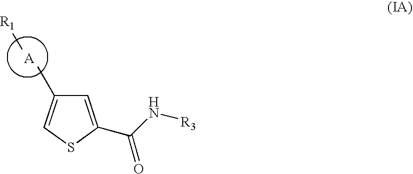
in which A, R₁ and R₃ are as defined in the formula (I),
their isomers and their addition salts with a pharmaceutically acceptable acid or base.

4- Compounds of formula (I) according to claim 2, characterized in that A represents a phenyl group, their isomeres and their addition salts with a pharmaceutically acceptable acid or base.

5- Compounds of formula (I) according to claim 2, characterized in that A represents a phenyl group, Rₐ represents a hydrogen atom, R_{b} represents a hydrogen atom, X represents an oxygen atom, Y represents an —NH— group, R₁ is as defined in the general formula (I), R₂ represents a hydrogen atom and R₃ represents a —U—R₁₁ group in which U and R₁₁ are as defined in the general definition of the formula (I), their isomers and their addition salts with a pharmaceutically acceptable acid or base.

6- Compounds of formula (I) according to claim 2, characterized in that the said A group taking the definition phenyl is substituted by an R₁ group as defined in the formula (I) situated in the para position, their isomers and their addition salts with a pharmaceutically acceptable acid or base.

7- Compounds of formula (I) according to claim 1, characterized in that R₁ represents a group selected from trifluoromethyl, trifluoromethoxy, 2,2,2-trifluoroethoxy, (C₁-C₆)alkyl, cyano, nitro, —OR₄, —SR₄, —NR₄R₅, —CO₂R₄, —C(O)R₄, -T-CO₂R₄, -T-OH, -T-CN, -T-R₆ and —R₆ in which: [symbol]
R₄ represents a hydrogen atom, a (C₁-C₆)alkyl, aryl or cycloalkyl group or a heterocycle, [symbol]
R₅ represents a hydrogen atom or a (C₁-C₆)alkyl group, [symbol]
R₆ represents a group selected from aryl and cycloalkyl and a heterocycle, each of these groups optionally being substituted by one or two identical or different groups selected from halogen, cyano, nitro, trifluoromethyl, halo(C₁-C₆)alkoxy, (C₁-C₆)alkyl, vinyl, —OR₄₀, —NR₄₀R₅₀, —S(O)ₙ₁R₄₀, —C(O)R₄₀, —CO₂R₄₀, —O—C(O)R₄₀, —C(O)NR₄₀R₅₀, —NR₅₀—C(O)R₄₀), —NR₅₀—SO₂R₄₀, -T₁-C(O)R₄₀, -T₁-CN, -T₁-OR₄₀ and -T₁-CO₂R₄₀, in which R₄₀, R₅₀, T₁ and n₁ are as defined in the formula (I), [symbol]
T represents a —CH₂— or —CH₂—O— group in which the oxygen atom is connected to the A group of the compounds of formula (I),
their isomers and their addition salts with a pharmaceutically acceptable acid or base.

8- Compounds of formula (I) according to claim 5, characterized in that R₁ represents a group as defined in claim 7, their isomers and their addition salts with a pharmaceutically acceptable acid or base.

9- Compounds of formula (I) according to claim 1, characterized in that R₁ represents a group selected from (C₂-C₄)alkyl, (C₁-C₄)alkylthio, (C₁-C₄)alkoxy, trifluoromethoxy and —R₆ in which R₆ represents a group selected from phenyl optionally substituted by one or two groups as defined in the formula (I), cyclohexyl and a 5- or 6-membered heterocycle comprising from 1 to 3 heteroatoms selected from oxygen, nitrogen and sulphur, each of the said R₆ groups optionally being substituted by a group as defined in the formula (I), their isomers and their addition salts with a pharmaceutically acceptable acid or base.

10- Compounds of formula (I) according to claim 1, characterized in that R₁ represents a group selected from:
phenyl optionally substituted by a group selected from halogen, hydroxyl, (C₁-C₄)alkoxy, phenoxy, trifluoromethoxy, acyl, (C₁-C₄)alkylsulphonyl, -T-CO₂R₄₀ and -T-CN in which T and R₄₀ are as defined in the formula (I),
cyclohexyl,
4-pyridyl, 3-pyridyl, 5-pyrimidyl, N-pyrrolidinyl, 1-methylpyrrol-3-yl, 3,6-dihydro-2H-pyridin-1-yl and 2-hydroxy-4-pyridyl,
their isomers and their addition salts with a pharmaceutically acceptable acid or base.

11- Compounds of formula (I) according to claim 5, characterized in that R₁ represents a group as defined in claim 10, their isomers and their addition salts with a pharmaceutically acceptable acid or base.

12- Compounds of formula (I) according to claim 1, characterized in that R₃ represents an R₇ group selected from phenyl, cyclohexyl and pyridyl, each of these groups optionally being substituted by one or two identical or different groups selected, independently of each other, from (C₁-C₆)alkyl, —OR₈, —NR₈R₉, —CO₂R₈, —V—OR₈, —V—NR₈R₉ and —V—CO₂R₈ in which V represents a linear or branched (C₁-C₄)alkylene chain or a linear or branched (C₂-C₄)alkenylene chain, R₈ represents a hydrogen atom or a (C₁-C₆)alkyl group, and R₉ represents a hydrogen atom, their isomers and their addition salts with a pharmaceutically acceptable acid or base.

13- Compounds of formula (I) according to claim 2, characterized in that R₃ represents a —U—R₁₁ group in which U represents a linear or branched (C₁-C₄)alkylene chain and R₁₁ represents a group selected from —CO₂R₁₂ and —R₁₄ in which: [symbol]
R₁₂ represents a hydrogen atom or a (C₁-C₆)alkyl group, [symbol]
R₁₄ represents a group selected from phenyl, cyclohexyl, morpholinyl and pyridyl, each of these groups optionally being substituted by one or two identical or different groups selected, independently of each other, from halogen, (C₁-C₆)alkyl, —CO₂R₁₅ and —W—CO₂R₁₅, in which R₁₅ represents a hydrogen atom or a (C₁-C₆)alkyl group and W represents a linear or branched (C₁-C₆)alkylene chain or a linear or branched (C₂-C₆)alkenylene chain,
their isomers and their addition salts with a pharmaceutically acceptable acid or base.

14- Compounds of formula (I) according to claim 1, which are:
4-(4-Isopropylphenyl)-N-(2-morpholin-4-ylethyl)thiophene-2-carboxamide
4-(4-Biphenyl)-N-(2-morpholin-4-ylethyl)thiophene-2-carboxamide
4-(4-Ethylphenyl)-N-(2-morpholin-4-ylethyl)thiophene-2-carboxamide
4-[4-(Methylthio)phenyl]-N-(2-morpholin-4-ylethyl)thiophene-2-carboxamide
4-[4-(Trifluoromethoxy)phenyl]-N-(2-morpholin-4-ylethyl)thiophene-2-carboxamide
4-[4-(tert-Butyl)phenyl]-N-(2-morpholin-4-ylethyl)thiophene-2-carboxamide
4-[4-(Trifluoromethyl)phenyl]-N-(2-morpholin-4-ylethyl)thiophene-2-carboxamide
4-(4-Hydroxyphenyl)-N-(2-morpholin-4-ylethyl)thiophene-2-carboxamide
4-[4-(Pyridin-4-yl)phenyl]-N-(2-morpholin-4-ylethyl)thiophene-2-carboxamide
Methyl 4′-[5-(2-morpholin-4-ylethylcarbamoyl)thiophen-3-yl]biphenyl-3-carboxylate
4-[4-(Pyridin-3-yl)phenyl]-N-(2-morpholin-4-ylethyl)thiophene-2-carboxamide
4-[4-(Morpholin-4-yl)phenyl]-N-(2-morpholin-4-ylethyl)thiophene-2-carboxamide
4-(4-Piperidinophenyl)-N-(2-morpholin-4-ylethyl)thiophene-2-carboxamide
4-[4-(Pyrrolidin-1-yl)phenyl]-N-(2-morpholin-4-ylethyl)thiophene-2-carboxamide
4-[4-( 1,4-Dioxa-8-azaspiro[4,5]dec-8-yl)phenyl]-N-(2-morpholin-4-ylethyl)thiophene-2-carboxamide
4-[4-(1,2,3,6-Tetrahydropyridin-1-yl)phenyl]-N-(2-morpholin-4-ylethyl)thiophene-2-carboxamide
4-[4-(3-(3R)-Hydroxypyrrolidin-1-yl)phenyl]-N-(2-morpholin-4-ylethyl)thiophene-2-carboxamide
4-[4-(1H-Imidazol-1-yl)phenyl]-N-(2-morpholin-4-ylethyl)thiophene-2-carboxamide
N-(2-Morpholin-4-ylethyl)-4-[4-(1H-pyrrol-1-yl)phenyl]thiophene-2-carboxamide
4-[4-(Isoxazol-5-yl)phenyl]-N-(2-morpholin-4-ylethyl)thiophene-2-carboxamide
4-[4-(Cyclohexyl)phenyl]-N-(2-morpholin-4-ylethyl)thiophene-2-carboxamide
4-[4-(1-Methyl-1H-pyrazol-3-yl)phenyl]-N-(2-morpholin-4-ylethyl)thiophene-2-carboxamide
4-[4-(6-Oxo-1,4,5,6-tetrahydropyridazin-3-yl)phenyl]-N-(2-morpholin-4-ylethyl)thiophene-2-carboxamide
Methyl trans-4-{[4-(4-phenylcyclohex-1-enyl)thiophene-2-carboxamido]methyl}-cyclohexanecarboxylate
trans 4-{[4-(4-Phenylcyclohex-1-enyl)thiophene-2-carboxamido]methyl}cyclohexane-carboxylic acid
Ethyl 3-(6-{[4-(4-trifluoromethoxyphenyl)thien-2-yl]carboxamido}pyridin-3-yl)-propanoate
3-(6-{[4-(4-Trifluoromethoxyphenyl)thien-2-yl]carboxamido}pyridin-3-yl)propanoic acid
Ethyl 3-(4-{[4-(4-trifluoromethoxyphenyl)thien-2-yl]carboxamido}phenyl)propenoate
3-(4-{[4-(4-Trifluoromethoxyphenyl)thien-2-yl]carboxamido}phenyl)propenoic acid
Diethyl 4-({[4-(trifluoromethoxyphenyl)thien-2-yl]carboxamido}methyl)phthalate
4-({[4-(Trifluoromethoxyphenyl)thien-2-yl]carboxamido}methyl)phthalic diacid
Ethyl 3-(4-{[4-(4-trifluoromethoxyphenyl)thien-2-yl]carboxamido}phenyl)propanoate
3-(4-{[4-(4-Trifluoromethoxyphenyl)thien-2-yl]carboxamido}phenyl)propanoic acid
Methyl trans-4-({[4-(4-trifluoromethoxyphenyl)thien-2-yl]carboxamido}methyl)-cyclohexanecarboxylate
trans-4-({[4-(4-Trifluoromethoxyphenyl)thien-2-yl]carboxamido}methyl)cyclohexane-carboxylic acid
Ethyl 2-[4-({[4-(4-trifluoromethoxyphenyl)thien-2-yl]carboxamido}methyl)phenyl]acetate
2-[4-({[4-(4-Trifluoromethoxyphenyl)thien-2-yl]carboxamido}methyl)phenyl]acetic acid
Ethyl 3-({[4-(4-trifluoromethoxyphenyl)thien-2-yl]carboxamido}methyl)benzoate
3-({[4-(4-Trifluoromethoxyphenyl)thien-2-yl]carboxamido}methyl)benzoic acid
Ethyl 2-[3-({[4-(4-trifluoromethoxyphenyl)thien-2-yl]carboxamido}methyl)phenyl]acetate
2-[3-({[4-(4-Trifluoromethoxyphenyl)thien-2-yl]carboxamido}methyl)phenyl]acetic acid
Ethyl 4-′{[4-(4-trifluoromethoxyphenyl)thien-2-yl]carboxamido}methyl)cyclohexane-carboxylate
4-({[4-(4-Trifluoromethoxyphenyl)thien-2-yl]carboxamido}methyl)cyclohexane carboxylic acid
Methyl 6-({[4-(4-trifluoromethoxyphenyl)thien-2-yl]carboxamido}methyl)nicotinate
6-({[4-(4-Trifluoromethoxyphenyl)thien-2-yl]carboxamido}methyl)nicotinic acid
Ethyl 4-({[4-(4-trifluoromethoxyphenyl)thien-2-yl]carboxamido}methyl)benzoate
4-({[4-(4-Trifluoromethoxyphenyl)thien-2-yl]carboxamido}methyl)benzoic acid
Ethyl 4-({[4-(4-methylthiophenyl)thien-2-yl]carboxamido}methyl)benzoate
4-({[4-(4-Methylthiophenyl)thien-2-yl]carboxamido}methyl)benzoic acid
Ethyl 4-({[4-(4-(tert-butyl)phenyl)thien-2-yl]carboxamido}methyl)benzoate
4-({[4-(4-(tert-Butyl)phenyl)thien-2-yl]carboxamido}methyl)benzoic acid
Ethyl (4-{[4-(4-(tert-butyl)phenyl)thien-2-yl]carboxamido}phenyl)acetate
(4-{[4-(4-(tert-Butyl)phenyl)thien-2-yl]carboxamido}phenyl)acetic acid
Ethyl (4-{[4-(4-trifluoromethoxyphenyl)thien-2-yl]carboxamido}phenyl)acetate
(4-{[4-(4-Trifluoromethoxyphenyl)thien-2-yl]carboxamido}phenyl)acetic acid
Ethyl (4-{[4-(4-methylthiophenyl)thien-2-yl]carboxamido}phenyl)acetate
(4-{[4-(4-Methylthiophenyl)thien-2-yl]carboxamido}phenyl)acetic acid
Ethyl 4-({[4-(4-methoxyphenyl)thien-2-yl]carboxamido}methyl)benzoate
4-({[4-(4-Methoxyphenyl)thien-2-yl]carboxamido}methyl)benzoic acid
Ethyl (4-{[4-(4-methoxyphenyl)thien-2-yl]carboxamido}phenyl)acetate
(4-{[4-(4-Methoxyphenyl)thien-2-yl]carboxamido}phenyl)acetic acid
Ethyl 3-[4-({[4-(4-trifluoromethoxyphenyl)thien-2-yl]carboxamido}methyl)phenyl]-propanoate
3-[4-({[4-(4-Trifluoromethoxyphenyl)thien-2-yl]carboxamido}methyl)phenyl]propanoic acid
Ethyl 3-[3-({[4-(4-trifluoromethoxyphenyl)thien-2-yl]carboxamido }methyl)phenyl]-propanoate
3-[3-({[4-(4-Trifluoromethoxyphenyl)thien-2-yl]carboxamido}methyl)phenyl]propanoic acid
Ethyl 7-{[4-(4-trifluoromethoxyphenyl)thien-2-yl]carboxamido}heptanoate
7-{[4-(4-Trifluoromethoxyphenyl)thien-2-yl]carboxamido}heptanoic acid
Ethyl 4-({[4-(4-hydroxyphenyl)thien-2-yl]carboxamido}methyl)cyclohexanecarboxylate
Ethyl 4-[({4-[4-(pyridin-4-yl)phenyl]thien-2-yl}carboxamido)methyl]cyclohexane-carboxylate
4-[({4-[4-(Pyridin-4-yl)phenyl]thien-2-yl]carboxamido)methyl]cyclohexanecarboxylic acid hydrochloride
4-(4-Bromophenyl)-N-(2-morpholin-4-ylethyl)thiophene-2-carboxamide,
4-[4-(4-Acetylphenyl)phenyl]-N-(2-morpholin-4-ylethyl)thiophene-2-carboxamide,
4-[4-(4-Fluorophenyl)phenyl]-N-(2-morpholin-4-ylethyl)thiophene-2-carboxamide,
4-[4-(3-Hydroxyphenyl)phenyl]-N-(2-morpholin-4-ylethyl)thiophene-2-carboxamide,
4-[4-(4-Methylsulphonylphenyl)phenyl]-N-(2-morpholin-4-ylethyl)thiophene-2-carboxamide,
4-[4-(3-Acetylphenyl)phenyl]-N-(2-morpholin-4-ylethyl)thiophene-2-carboxamide,
N-(2-Morpholin-4-ylethyl)-4-[4-(trifluoromethoxy)phenyl]thiophene-2-carbothioamide,
N-(2-Morpholin-4-ylethyl)-4-[4-(1,2,3-thiadiazol-4-yl)phenyl]thiophene-2-carboxamide,
4-(4-Isoxazol-5-ylphenyl)-N-(2-morpholin-4-ylethyl)thiophene-2-carboxamide,
trans-[4-([4-(4-Trifluoromethoxyphenyl)thien-2-yl]carboxamido)cyclohexyl]acetic acid,
2-[4-({[4-(4-Phenoxyphenyl)thien-2-yl]carboxamido}methyl)phenyl]acetic acid,
4-(4-Trifluoromethoxyphenyl)-N-methylthiophene-2-carboxamide,
2-[4-({[4-(4-Benzyloxyphenyl)thien-2-yl]carboxamido}methyl)phenyl]acetic acid,
N-(3,5-Difluoro-4-hydroxybenzyl)-4-(4-trifluoromethoxyphenyl)thiophene-2-carboxamide,
4-[4-(3-Nitrophenyl)phenyl]-N-(2-morpholin-4-ylethyl)thiophene-2-carboxamide,
4-[4-(2-Chlorophenyl)phenyl]-N-(2-morpholin-4-ylethyl)thiophene-2-carboxamide,
4-[4-(3-Cyanophenyl)phenyl]-N-(2-morpholin-4-ylethyl)thiophene-2-carboxamide,
4-[4-(2-Formylphenyl)phenyl]-N-(2-morpholin-4-ylethyl)thiophene-2-carboxamide,
4-[4-(3,4,5-Trimethoxyphenyl)phenyl]-N-(2-morpholin-4-ylethyl)thiophene-2-carboxamide,
4-{4-[4-(Hydroxymethyl)phenyl]phenyl}-N-(2-morpholin-4-ylethyl)thiophene-2-carboxamide,
4-[4-(3-Acetamidophenyl)phenyl-N-(2-morpholin-4-ylethyl)thiophene-2-carboxamide,
4-{4-[3,4-(Methylenedioxy)phenyl]phenyl}-N-(2-morpholin-4-ylethyl)thiophene-2-carboxamide,
N-(Cyclopropylmethyl)-4-[4-(trifluoromethoxy)phenyl]thiophene-2-carboxamide,
N-(tert-Butyl)-4-[4-(trifluoromethoxy)phenyl]thiophene-2-carboxamide,
N-(Cyclopropyl)-4-[4-(trifluoromethoxy)phenyl]thiophene-2-carboxamide,
N-(Cyclopentyl)-4-[4-(trifluoromethoxy)phenyl]thiophene-2-carboxamide,
N-(2-Hydroxyethyl)-4-[4-(trifluoromethoxy)phenyl]thiophene-2-carboxamide,
N-(Cyclopentylmethyl)-4-[4-(trifluoromethoxy)phenyl]thiophene-2-carboxamide,
trans-4-({[4-(4-Trifluoromethoxyphenyl)thien-2-yl]carboxamido}-methyl)cyclohexanecarboxylic acid, sodium salt
4-{4-[(3-Chloro-4-fluoro)phenyl]phenyl}-N-(2-morpholin-4-ylethyl)thiophene-2-carboxamide,
4-{4-[3-(Hydroxymethyl)phenyl]phenyl}-N-(2-morpholin-4-ylethyl)thiophene-2-carboxamide,
Ethyl 2-[4-({[4-(4′-propionyl-4-biphenyl)thien-2-yl]carboxamido}methyl)phenyl]acetate
2-[4-({[4-(4′-Propionyl-4-biphenyl)thien-2-yl]carboxamido}methyl)phenyl]acetic acid,
Ethyl 2-[4-({[4-(4′-cyclopropylcarbonyl-4-biphenyl)thien-2-yl]carboxamido}methyl) phenyl acetate,
2-[4-({[4-(4′-Cyclopropylcarbonyl-4-biphenyl)thien-2-yl]carboxamido}methyl)phenyl]-acetic acid,
Ethyl 2-[4-({[4-(4′-benzoyl-4-biphenyl)thien-2-yl]carboxamido}methyl)phenyl]acetate,
2-[4-({[4-(4′-Benzoyl-4-biphenyl)thien-2-yl]carboxamido}methyl)phenyl]acetic acid,
Ethyl 2-[4-({[4-(4′-cyanomethyl-4-biphenyl)thien-2-yl]carboxamido}methyl)phenyl]acetate,
2-[4-({[4-(4′-Cyanomethyl-4-biphenyl)thien-2-yl]carboxamido}methyl)phenyl]acetic acid,
Ethyl 2-{4-[({4-[4-(pyrimidin-5-yl)phenyl]thien-2-yl}carboxamido)methyl]phenyl}acetate,
2-{4-[({4-[4-(Pyrimidin-5-yl)phenyl]thien-2-yl}carboxamido)methyl]phenyl}acetate acid,
Ethyl 2-{4-[({4-[4-(4-acetyl-3-hydroxyphenyl)phenyl]thien-2-yl}carboxamido)methyl]phenyl}acetate,
2-{4-[({4-[4-(4-Acetyl-3-hydroxyphenyl)phenyl]thien-2-yl}carboxamido)methyl]phenyl}-acetic acid,
Ethyl 2-{4-[({4-[4-(2-hydroxyphenyl)phenyl]thien-2-yl}carboxamido)methyl]phenyl}acetate,
2-{4-[({4-[4-(2-Hydroxyphenyl)phenyl]thien-2-yl}carboxamido)methyl]phenyl}acetic acid,
Ethyl 2-{4-[({4-[4-(4-acetyl-2-methoxyphenyl)phenyl]thien-2-yl}carboxamido)methyl]phenyl}acetate,
2-{4-[({4-[4-(4-Acetyl-2-hydroxyphenyl)phenyl]thien-2-yl}carboxamido)methyl]phenyl}-acetic acid,
Ethyl 2-{4-[({4-[4-(4-(2-oxopropyl)-3-hydroxyphenyl)phenyl]thien-2-yl}carboxamido) methyl]phenyl}acetate,
2-{4-[({4-[4-(4-(2-Oxopropyl)-3-hydroxyphenyl)phenyl]thien-2-yl}carboxamido)methyl]-phenyl}acetic acid,
4-[4-(2-Fluorophenyl)phenyl]-N-(2-morpholin-4-ylethyl)thiophene-2-carboxamide,
N-[2-(Hydroxymethyl)cyclohexyl]-4-(4-trifluoromethoxyphenyl)thiophene-2-carboxamide,
Ethyl trans-4-[({4-[4-(4-acetylphenyl)phenyl]thien-2-yl}carboxamido)methyl]cyclohexanecarboxylate,
trans-4-[({4-[4-(4-Acetylphenyl)phenyl]thien-2-yl}carboxamido)methyl]cyclohexane-carboxylic acid,
Methyl trans-3-(3-{[4-(4-trifluoromethoxyphenyl)thien-2-yl]carboxamido}cyclohexyl) propanoate,
trans-3-(3-{[4-(4-Trifluoromethoxyphenyl)thien-2-yl]carboxamido}cyclohexyl)propanoic acid,
Methyl trans-(3-{[4-(4-trifluoromethoxyphenyl)thien-2-yl]carboxamido}cyclohexyl) acetate,
trans-(3-{[4-(4-Trifluoromethoxyphenyl)thien-2-yl]carboxamido}cyclohexyl)acetic acid,
Methyl trans-3-(4-{[4-(4-trifluoromethoxyphenyl)thien-2-yl]carboxamido}cyclohexyl) propanoate,
trans-3-(4-{[4-(4-Trifluoromethoxyphenyl)thien-2-yl]carboxamido}cyclohexyl)propanoic acid,
Methyl [4-({[4-(4-Trifluoromethoxyphenyl)thien-2-yl]carboxamido}methyl)cyclohexyl]acetate,
[4-({[4-(4-Trifluoromethoxyphenyl)thien-2-yl]carboxamido}methyl)cyclohexyl]acetic acid,
Ethyl 2-hydroxy-5-({[4-(4-trifluoromethoxyphenyl)thien-2-yl]carboxamido}methyl) benzoate,
2-Hydroxy-5-({[4-(4-trifluoromethoxyphenyl)thien-2-yl]carboxamido}methyl)benzoic acid,
Methyl 6-({[4-(4′-acetyl-4-biphenyl)thien-2-yl]carboxamido}methyl)nicotinate,
6-({[4-(4′-Acetyl-4-biphenyl)thien-2-yl]carboxamido}methyl)nicotinic acid,
N-(Pyridin-4-yl)-4-(4-trifluoromethoxyphenyl)thiophene-2-carboxamide,
N-(2-Hydroxy-1-hydroxymethyl-1-methylethyl)-4-(4-trifluoromethoxyphenyl)thiophene-2-carboxamide,
N-(2-Hydroxypropyl)-4-(4-trifluoromethoxyphenyl)thiophene-2-carboxamide,
N-(2-Hydroxy-1,1-dimethylethyl)-4-(4-trifluoromethoxyphenyl)thiophene-2-carboxamide,
4-(4-Trifluoromethoxyphenyl)-N-(tetrahydrofuran-2-ylmethyl)thiophene-2-carboxamide,
N-(2,2-Dimethyl-1,3-dioxolan-4-ylmethyl)-4-(4-trifluoromethoxyphenyl)thiophene-2-carboxamide,
Ethyl 3-[4-(4-trifluoromethoxyphenyl)thiophene-2-carboxamido]butyrate,
3-[4-(4-Trifluoromethoxyphenyl)thiophene-2-carboxamido]butyric acid,
N-[3-(Methylsulphinyl)propyl]-4-(4-trifluoromethoxyphenyl)thiophene-2-carboxamide,
N-(2-Morpholin-4-ylethyl)-4-[4-(2,2,2-trifluoroethoxy)phenyl]thiophene-2-carboxamide,
{4′-[5-(2-Morpholin-4-ylethylcarbamoyl)thiophen-3-yl]biphenyl-4-yl}acetic acid, sodium salt,
Ethyl trans-4-({[4-[4-(trifluoromethoxy)phenyl]thien-2-yl]carbothioamido}methyl)-cyclohexanecarboxylate,
trans-4-({[4-[4-(trifluoromethoxy)phenyl]thien-2-yl]carbothioamido}methyl)-cyclohexane carboxylic acid,
Ethyl (2S)-2-{[4-(4-trifluoromethoxyphenyl)thien-2-yl]carboxamido}-3-methylbutanoate,
(2S)-2-{[4-(4-trifluoromethoxyphenyl)thien-2-yl]carboxamido}-3-methylbutanoic acide,
and N-(2-Morpholin-4-ylethyl)-4-(5-phenylpyridin-2-yl)thiophen-2-carboxamide.

15- Compounds of formula (I) according to claim 1, which are:
4-(4-Biphenyl)-N-(2-morpholin-4-ylethyl)thiophene-2-carboxamide,
4-[4-(Pyridin-4-yl)phenyl]-N-(2-morpholin-4-ylethyl)thiophene-2-carboxamide,
4-[4-(Pyridin-3-yl)phenyl]-N-(2-morpholin-4-ylethyl)thiophene-2-carboxamide,
4-[4-(Pyrrolidin-1-yl)phenyl]-N-(2-morpholin-4-ylethyl)thiophene-2-carboxamide,
4-[4-(Cyclohexyl)phenyl]-N-(2-morpholin-4-ylethyl)thiophene-2-carboxamide,
4-[4-(1-Methyl-1H-pyrazol-3-yl)phenyl]-N-(2-morpholin-4-ylethyl)thiophene-2-carboxamide,
3-(6-{[4-(4-Trifluoromethoxyphenyl)thien-2-yl]carboxamidopyridin-3-yl)propanoic acid,
3-(4-{[4-(4-Trifluoromethoxyphenyl)thien-2-yl]carboxamido}phenyl)propanoic acid,
trans-4-({[4-(4-Trifluoromethoxyphenyl)thien-2-yl]carboxamido}methyl)cyclohexane-carboxylic acid,
2-[4-({[4-(4-Trifluoromethoxyphenyl)thien-2-yl]carboxamido[methyl)phenyl]acetic acid,
2-[3-({[4-(4-Trifluoromethoxyphenyl)thien-2-yl]carboxamido{methyl)phenyl]acetic acid,
4-{[4-(4-Trifluoromethoxyphenyl)thiophene-2-carboxamido]methyl}cyclohexane-carboxylic acid,
6-({[4-(4-Trifluoromethoxyphenyl)thien-2-yl]carboxamido}methyl)nicotinic acid,
4-({[4-(4-Trifluoromethoxyphenyl)thien-2-yl]carboxamido}methyl)benzoic acid,
(4-{[4-(4-Trifluoromethoxyphenyl)thien-2-yl]carboxamido}phenyl)acetic acid,
3-[4-({[4-(4-Trifluoromethoxyphenyl)thien-2-yl]carboxamido}methyl)phenyl]-propanoic acid,
3-[3-({[4-(4-Trifluoromethoxyphenyl)thien-2-yl]carboxamido}methyl)phenyl]-propanoic acid,
4-[({4-[4-(Pyridin-4-yl)phenyl]thien-2-yl}carboxamido)methyl]cyclohexanecarboxylic acid hydrochloride,
4-[4-(4-Acetylphenyl)phenyl]-N-(2-morpholin-4-ylethyl)thiophene-2-carboxamide,
4-[4-(4-Fluorophenyl)phenyl]-N-(2-morpholin-4-ylethyl)thiophene-2-carboxamide,
4-[4-(3-Hydroxyphenyl)phenyl]-N-(2-morpholin-4-ylethyl)thiophene-2-carboxamide,
4-[4-(4-Methylsulphonylphenyl)phenyl]-N-(2-morpholin-4-ylethyl)thiophene-2-carboxamide,
4-[4-(3-Acetylphenyl)phenyl]-N-(2-morpholin-4-ylethyl)thiophene-2-carboxamide,
2-[4-({[4-(4-phenoxyphenyl)thien-2-yl]carboxamido}methyl)phenyl]acetic acid,
N-(3,5-Difluoro-4-hydroxybenzyl)-4-(4-trifluoromethoxyphenyl)thiophene-2-carboxamide,
4-[4-(4-Hydroxymethylphenyl)phenyl]-N-(2-morpholin-4-ylethyl)thiophene-2-carboxamide,
trans-4-({[4-(4-Trifluoromethoxyphenyl)thien-2-yl]carboxamido}-methyl)cyclohexanecarboxylic acid, sodium salt
4-[4-(3-Hydroxymethylphenyl)phenyl]-N-(2-morpholin-4-ylethyl)thiophene-2-carboxamide,
2-[4-({[4-(4′-Propionyl-4-biphenyl)thien-2-yl]carboxamido}methyl)phenyl]acetic acid,
2-[4-({[4-(4′-Cyclopropylcarbonyl-4-biphenyl)thien-2-yl]carboxamido}methyl)-phenyl]acetic acid,
2-[4-({[4-(4′-Benzoyl-4-biphenyl)thien-2-yl]carboxamido}methyl)phenyl]acetic acid,
2-[4-({[4-(4 ′-Cyanomethyl-4-biphenyl)thien-2-yl]carboxamido}methyl)phenyl]acetic acid,
2-{4-[({4-[4-(Pyrimidin-5-yl)phenyl]thien-2-yl}carboxamido)methyl]phenyl}acetic acid,
2-{4-[({4-[4-(4-Acetyl-3-hydroxyphenyl)phenyl]thien-2-yl}carboxamido)methyl]-phenyl}acetic acid,
2-{4-[({4-[4-(2-Hydroxyphenyl)phenyl]thien-2-yl}carboxamido)methyl]phenyl}acetic acid,
2-{4-[({4-[4-(4-Acetyl-2-hydroxyphenyl)phenyl]thien-2-yl}carboxamido)methyl]-phenyl}acetic acid,
N-[2-(Hydroxymethyl)cyclohexyl]-4-(4-trifluoromethoxyphenyl)thiophene-2-carboxamide,
trans-4-[({4-[4-(4-Acetylphenyl)phenyl]thien-2-yl}carboxamido)methyl]cyclohexane-carboxylic acid,
trans-3-(3-{[4-(4-Trifluoromethoxyphenyl)thien-2-yl]carboxamido}cyclohexyl)-propanoic acid,
trans-3-(4-{[4-(4-Trifluoromethoxyphenyl)thien-2-yl]carboxamido}cyclohexyl)-propanoic acid,
[4-({[4-(4-Trifluoromethoxyphenyl)thien-2-yl]carboxamido}methyl)cyclohexyl]acetic acid,
2-Hydroxy-5-({[4-(4-trifluoromethoxyphenyl)thien-2-yl]carboxamido}methyl)benzoic acid,
6-({[4-(4′-Acetyl-4-biphenyl)thien-2-yl]carboxamido}methyl)nicotinic acid,
and N-(Pyridin-4-yl)-4-(4-trifluoromethoxyphenyl)thiophene-2-carboxamide.

16- Compounds of formula (I) according to claim 1, which are:
4-[({4-[4-(Pyridin-4-yl)phenyl]thien-2-yl}carboxamido)methyl]cyclohexanecarboxylic acid hydrochloride,
4-[4-(4-Acetylphenyl)phenyl]-N-(2-morpholin-4-ylethyl)thiophene-2-carboxamide,
2-[4-({[4-(4′-Propionyl-4-biphenyl)thien-2-yl]carboxamido}methyl)phenyl]acetic acid,
2-[4-({[4-(4′-Cyclopropylcarbonyl-4-biphenyl)thien-2-yl]carboxamido}methyl)-phenyl]acetic acid,
2-[4-({[4-(4′-Benzoyl-4-biphenyl)thien-2-yl]carboxamido}methyl)phenyl]acetic acid,
trans-4-[({4-[4-(4-Acetylphenyl)phenyl]thien-2-yl}carboxamido)methyl]cyclohexane-carboxylic acid,
and 6-({[4-(4′-Acetyl-4-biphenyl)thien-2-yl]carboxamido}methyl)nicotinic acid.

17- Process for the preparation of the compounds of formula (I), characterized in that use is made, as starting material, of a compound of formula (II): 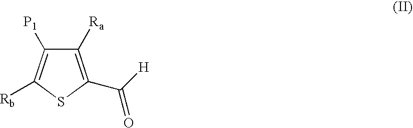
in which Rₐ and R_{b} are as defined in the formula (I) and P₁ represents a halogen atom or a triflate group,
which compounds of formula (II) are subjected to oxidation conditions in the presence, for example, of silver nitrate in a basic medium, to give the compounds of formula (III): 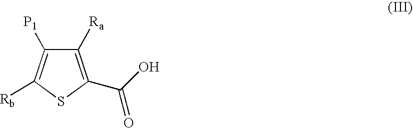
in which Rₐ, R_{b} and P₁ are as defined above,
which compounds of formula (III) are optionally converted to the corresponding acid chlorides (IV) by reaction with oxalyl chloride, for example, 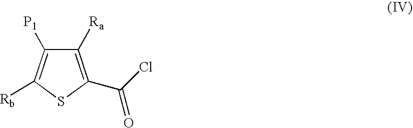
in which Rₐ, R_{b} and P₁ are as defined above,
or which compounds of formula (III) are treated directly, under peptide coupling conditions in the presence of a coupling agent and in a basic and polar medium, with a compound of formula (V):
HY—R₃ (V)
in which Y and R₃ have the same meanings as in the compounds of formula (I), to yield to the compounds of formula (VI): 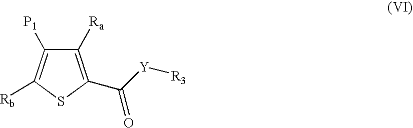
in which Rₐ, R_{b}, R₃, Y and P₁ are as defined above,
which compounds of formula (VI) are:
either reacted, under basic palladium coupling conditions, with a compound of formula (VII): 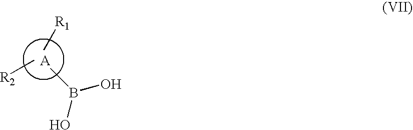
in which A, R₁ and R₂ have the same meanings as in the compounds of formula (I), to yield to the compounds of formula (I/a), a particular case of the compounds of formula (I): 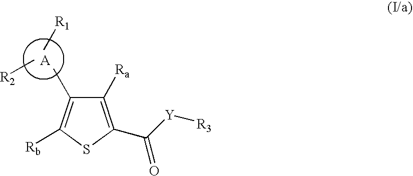
in which Rₐ, R_{b}, R₁, R₂, R₃, Y and A are as defined above,
or treated with hexamethylditin, in the presence of a palladium catalyst, to yield to the compounds of formula (VIII): 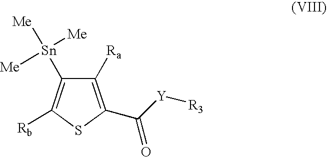
in which Rₐ, R_{b}, R₃ and Y are as defined above,
which componds of formula (VIII) are reacted:
with a compound of formula (IX): 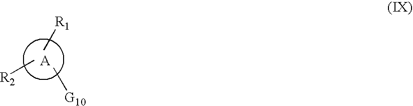
in which R₁, R₂ and A have the same meanings as in the formula (I) and G₁₀ represents a halogen atom selected from chlorine and bromine or a triflate group,
either in the presence of triphenylphosphinearsenic and of a palladium catalyst, in the case where G₁₀ represents a triflate group,
or in the presence of a cupric halide compound, such as CuBr₂, and of a palladium catalyst, under polar solvent conditions, in the case where G₁₀ represents a halogen atom,
to also yield to the compounds of formula (I/a) as described above,
or treated with bis(pinacolato)diborane, followed by an oxidation reaction, to yield to the compounds of formula (VIa): 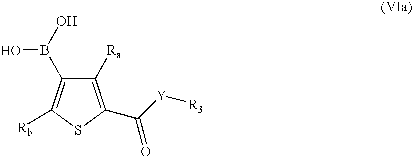
in which Rₐ, R_{b}, R₃ and Y are as defined above,
which compounds of formula (VIa) are reacted, under basic palladium coupling conditions, with a compound of the formula (IX): 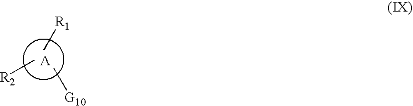
in which A, R₁, R₂ and G₁₀ are as defined above,
to also yield to the compounds of formula (I/a) as defined above: 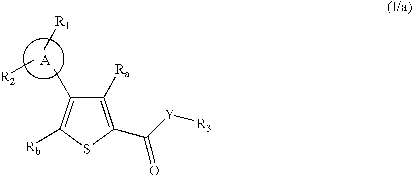
in which Rₐ, R_{b}, R₁, R₂, R₃, Y and A are as defined above,
or reacted with a compound of formula (IXa): 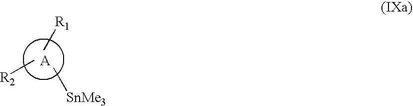
in which A, R₁, and R₂ are as defined in the formula (I),
either in the presence of triphenylphosphinearsenic and of a palladium catalyst, in the case where PI in the compounds of formula (VI) represents a triflate group,
or in the presence of a cupric halide compound, such as CuBr₂, and of a palladium catalyst, under polar solvent conditions, in the case where P₁ in the compounds of formula (VI) represents a halogen atom,
to also yield to the compounds of formula (I/a) as described above, 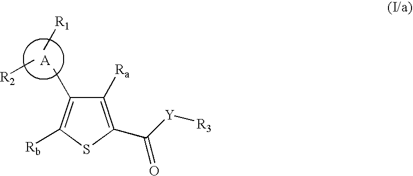
in which Rₐ, R_{b}, R₁, R₂, R₃, Y and A are as defined above,
which compounds of formula (I/a), in the specific case where they represent compounds of formula (I/b), in which A represents a phenyl group, R₂ represents a hydrogen atom, R₁ represents a hydroxyl group or a halogen atom (Hal) and Rₐ, R_{b}, Y and R₃ have the same meanings as in the formula (I): 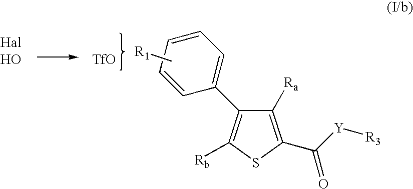
can then be treated beforehand with trifluoromethanesulphonic anhydride in the presence of a strong base, in the case where R₁ represents a hydroxyl group, to produce the triflate activated derivative,
it then being possible for the said compounds carrying an R₁ group taking the definition halogen or triflate:
either to be reacted under basic conditions and in the presence of a palladium catalyst with a compound of formula (X): 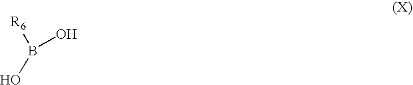
in which R₆ is as defined in the formula (I), that is to say that it represents a group selected from aryl and cycloalkyl and a heterocycle, each optionally being substituted,
to yield to the compounds of formula (I/c), a particular case of the compounds of formula (I): 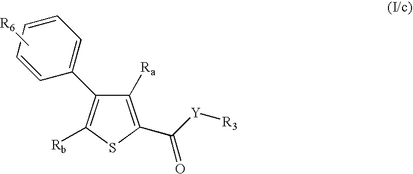
in which R₆, Rₐ, R_{b}, Y and R₃ are as defined above,
or to be treated with bis(pinacolato)diborane, followed by an oxidation reaction, to yield to the compounds of formula (XI): 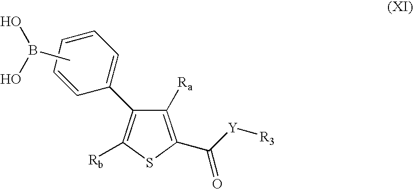
in which Rₐ, R_{b}, Y and R₃ are as defined above,
which compounds of formula (XI) are reacted under basic conditions and in the presence of a palladium catalyst with a compound of formula (Xa):
R₆—P₂ (Xa)
in which R₆ is as defined in the formula (I), that is to say that it represents a group selected from aryl and cycloalkyl and a heterocycle, each optionally being substituted, and P₂ represents a halogen atom or a triflate group,
to also yield to the compounds of formula (I/c) as defined above: 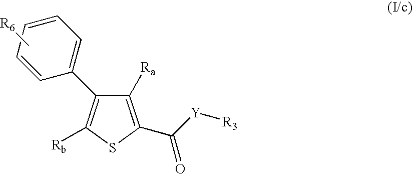
in which R₆, Rₐ, R_{b}, Y and R₃ are as defined above,
or to be treated with hexamethylditin in the presence of a palladium catalyst, to yield to the compounds of formula (XIa): 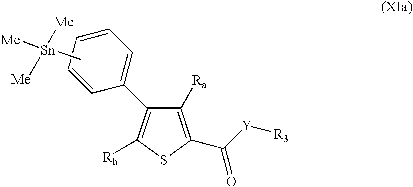
in which Rₐ, R_{b}, R₃ and Y are as defined above,
which compounds of formula (XIa) are reacted with a compound of formula (Xa) as defined above:
R₆—P₂ (Xa)
in which R₆ is as defined in the formula (I) and P₂ represents a halogen atom or a triflate group,
either in the presence of triphenylphosphinearsenic and of a palladium catalyst, in the case where P₂ represents a triflate group,
or in the presence of a cupric halide compound, such as CuBr₂, and of a palladium catalyst, under polar solvent conditions, in the case where P₂ represents a halogen atom,
to also yield to the compounds of formula (I/c) as defined above: 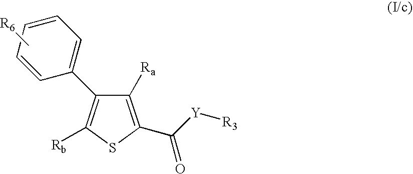
in which R₆, Rₐ, R_{b}, Y and R₃ are as defined above,
or to be reacted with a compound of formula (Xb):
R₆—SnMe₃ (Xb)
in which R₆ is as defined above,
either in the presence of triphenylphosphinearsenic and of a palladium catalyst, in the case where the compounds of formula (I/b) comprise a triflate group,
or in the presence of a cupric halide compound, such as CuBr₂, and of a palladium catalyst, under polar solvent conditions, in the case where the compounds of formula (I/b) comprise a halogen atom,
to also yield to the compounds of formula (I/c) as described above, 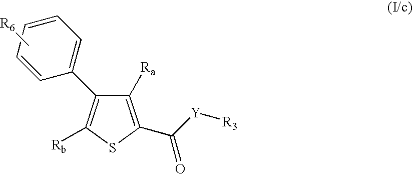
in which R₆, Rₐ, R_{b}, Y and R₃ are as defined above,
or to be reacted under palladium coupling conditions, in a basic medium, with a compound of formula (XII):
R₆—H (XII)
in which R_{6′} represents a nitrogenous heterocycle optionally substituted by one or more groups such as are defined for the substituents of the R₆ group within the compounds of formula (I),
to yield to the compounds of formula (I/d), a particular case of the compounds of formula (I): 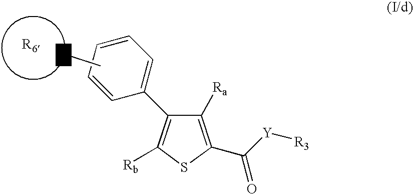
in which Rₐ, R_{b}, Y and R₃ are as defined above and R_{6,} represents an optionally substituted nitrogenous heterocycle as defined in the formula (I),
the compounds of formulae (I/a), (I/b), (I/c) and (I/d) together forming the compounds of formula (I/e): 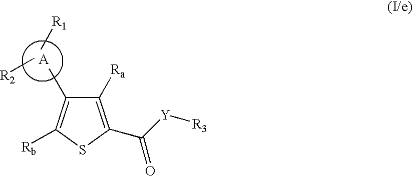
in which Rₐ, R_{b}, R₁, R₂, A, Y and R₃ are as defined in the formula (I),
which compounds of formula (I/e) can be treated, for example with 2,4-bis(4-methoxyphenyl)-1,3-dithia-2,4-diphosphetane-2,4-disulphide, to yield to the compounds of formula (I/f), a particular case of the compounds of formula (I): 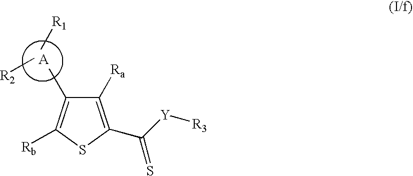
in which Rₐ, R_{b}, R₁, R₂, A, Y and R₃ are as defined in the formula (I),
the compounds (I/a) to (I/f) forming all the compounds of the invention, which are purified, if appropriate, according to a conventional purification technique, which can, if desired, be separated into their various isomers according to a conventional separating technique, and which are converted, if appropriate, into their addition salts with a pharmaceutically acceptable acid or base.

18- Process for the preparation of the compounds of formula (I), characterized in that use is made, as starting material, of a compound of formula (II): 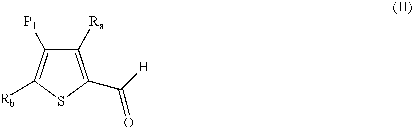
in which Rₐ and R_{b} are as defined in the formula (I) and P₁ represents a halogen atom or a triflate group,
which compounds of formula (II) are subjected to oxidation conditions in a basic and polar medium, to give the compounds of formula (III): 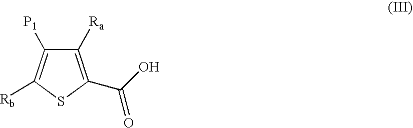
in which Rₐ, R_{b} and P₁ are as defined above,
the acid functional group of compounds of formula (III) is esterified by the action of an alcohol in the presence of a strong acid, to yield to the compounds of formula (XX): 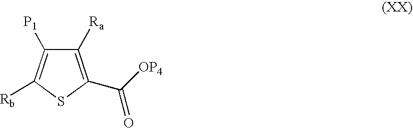
in which Rₐ, R_{b} and P₁ are as defined above and P₄ represents a linear or branched (C₁-C₄)alkyl group,
which compounds of formula (XX) are:
either reacted, under basic palladium coupling conditions, with a compound of formula (VII): 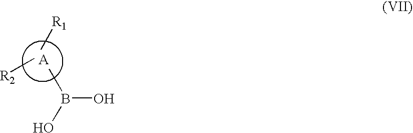
in which A, R₁ and R₂ have the same meanings as in the compounds of formula (I),
to yield to the compounds of formula (I/g), a particular case of the compounds of formula (I): 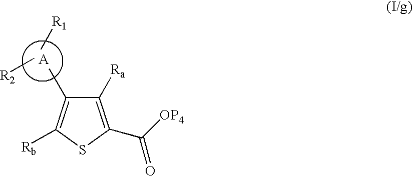
in which Rₐ, R_{b}, R₁, R₂, A and P₄ are as defined above,
or treated with hexamethylditin in the presence of a palladium catalyst, to yield to the compounds of formula (XXI): 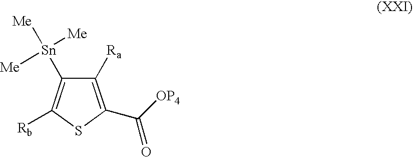
in which Rₐ, R_{b} and P₄ are as defined above,
which compounds of formula (XXI) are reacted:
with a compound of formula (IX): 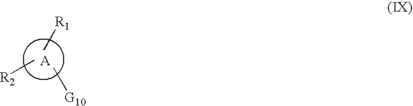
in which R₁, R₂ and A have the same meanings as in the formula (I) and G₁₀ represents a halogen atom selected from chlorine and bromine or a triflate group,
either in the presence of triphenylphosphinearsenic and of a palladium catalyst, in the case where G₁₀ represents a triflate group,
or in the presence of a cupric halide compound, such as CuBr₂, and of a palladium catalyst, under polar solvent conditions, in the case where G₁₀ represents a halogen atom,
to also yield to the compounds of formula (I/g) as described above,
or treated with bis(pinacolato)diborane, followed by an oxidation reaction, to yield to the compounds of formula (XXII): 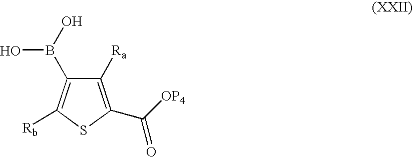
in which Rₐ, R_{b} and P₄ are as defined above,
which compounds of formula (XXII) are reacted under basic palladium coupling conditions with a compound of formula (IX): 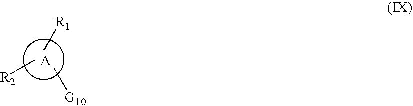
in which R₁, R₂, G₁₀ and A are as defined above,
to also yield to the compounds of formula (I/g) as defined above: 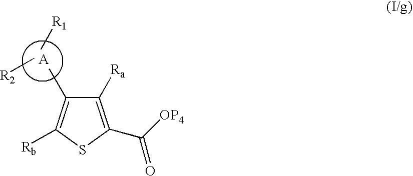
in which Rₐ, R_{b}, R₁, R₂ and P₄ are as defined above,
or reacted with a compound of formula (IXa): 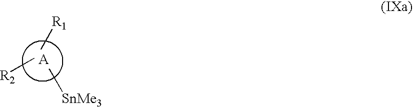
in which A, R₁ and R₂ are as defined in the formula (I),
either in the presence of triphenylphosphinearsenic and of a palladium catalyst, in the case where P₁ in the compounds of formula (XX) represents a triflate group,
or in the presence of a cupric halide compound, such as CuBr₂, and of a palladium catalyst, under polar solvent conditions, in the case where P₁ in the compounds of formula (XX) represents a halogen atom,
to also yield to the compounds of formula (I/g) as described above,
which compounds of formula (I/g), in the specific case where they represent compounds of formula (I/h), in which A represents a phenyl group, R₂ represents a hydrogen atom, R₁ represents a hydroxyl group or a halogen atom (Hal) and Rₐ, R_{b} and P₄ have the same meaning as in the formula (I): 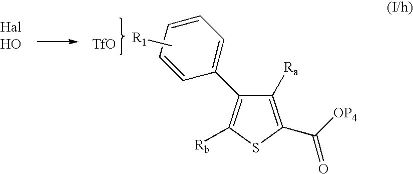
can then be treated beforehand with trifluoromethanesulphonic anhydride in the presence of a strong base, in the case where R₁ represents a hydroxyl group, to produce the triflate activated derivative,
it then being possible for the said compounds carrying an R₁ group taking the definition halogen or triflate:
either to be reacted, under basic conditions and in the presence of a palladium catalyst, with a compound of formula (X): 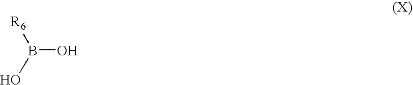
in which R₆ is as defined in the formula (I), that is to say that it represents a group selected from aryl and cycloalkyl and a heterocycle, each optionally being substituted,
to yield to the compounds of formula (I/i), a particular case of the compounds of formula (I): 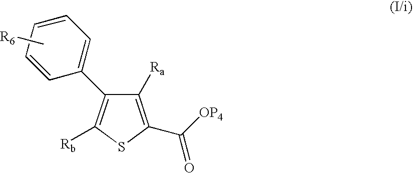
in which R₆, Rₐ, R_{b} and P₄ are as defined above,
or to be treated with bis(pinacolato)diborane, followed by an oxidation reaction, to yield to the compounds of formula (XXIII): 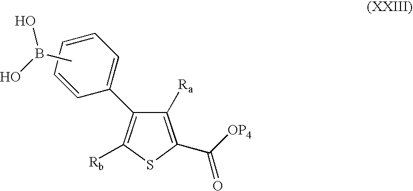
in which Rₐ, R_{b} and P₄ are as defined above,
which compounds of formula (XXIII) are reacted, under basic conditions and in the presence of a palladium catalyst, with a compound of formula (Xa):
R₆—P₂ (Xa)
in which R₆ is as defined in the formula (I), that is to say that it represents a group selected from aryl and cycloalkyl and a heterocycle, each optionally being substituted, and P₂ represents a halogen atom or a triflate group,
to also yield to the compounds of formula (I/i) as defined above: 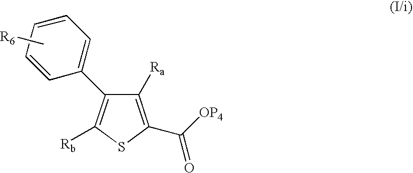
in which R₆, Rₐ, R_{b} and P₄ are as defined above,
or to be treated with hexamethylditin, in the presence of a palladium catalyst, to yield to the compounds of formula (XXIV): 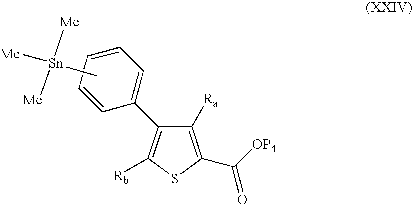
in which Rₐ, R_{b} and P₄ are as defined above,
which compounds of formula (XXIV) are reacted with a compound of formula (Xa) as defined above:
R₆—P₂ (Xa)
in which R₆ is as defined in the formula (I) and P₂ represents a halogen atom or a triflate group,
either in the presence of triphenylphosphinearsenic and of a palladium catalyst, in the case where P₂ represents a triflate group,
or in the presence of a cupric halide compound, such as CuBr₂, and of a palladium catalyst, under polar solvent conditions, in the case where P₂ represents a halogen atom,
to also yield to the compounds of formula (I/i) as defined above: 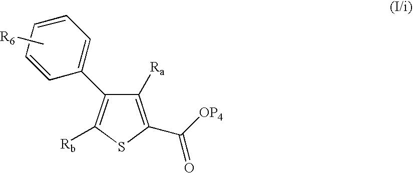
in which R₆, Rₐ, R_{b} and P₄ are as defined above,
or to be reacted with a compound of formula (Xb):
R₆—SnMe₃ (Xb)
in which R₆ is as defined above,
either in the presence of triphenylphosphinearsenic and of a palladium catalyst, in the case where the compounds of formula (I/h) comprise a triflate group,
or in the presence of a cupric halide compound, such as CuBr₂, and of a palladium catalyst, under polar solvent conditions, in the case where the compounds of formula (I/h) comprise a halogen atom,
to also yield to the compounds of formula (I/i) as described above, 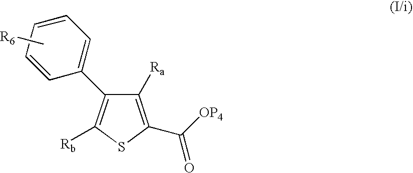
in which R₆, Rₐ, R_{b} and P₄ are as defined above,
the compounds of formulae (I/g), (I/h) and (I/i) together forming the compounds of formula (I/j): 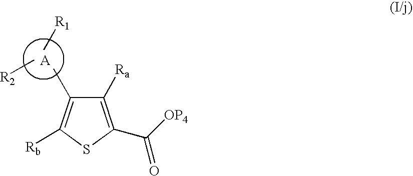
in which Rₐ, R_{b}, R₁, R₂, A and P₄ are as defined above,
which compounds of formula (I/j) are saponified under basic hydrolysis conditions to yield to the compounds of formula (I/k), a particular case of the compounds of formula (I): 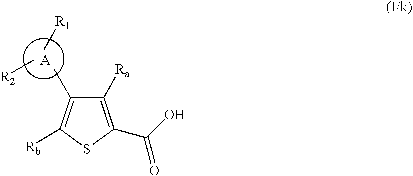
in which Rₐ, R_{b}, R₁, R₂ and A are as defined in the formula (I),
which compounds of formula (I/k) are either activated beforehand to the acid chloride by reaction in particular with oxalyl chloride or treated directly, under peptide coupling conditions in the presence, for example, of a coupling agent and in a basic and nonpolar medium, with a compound of formula (V):
HY—R₃ (V)
in which Y and R₃ have the same meanings as in the compounds of formula (I), to yield to the compounds of formula (I/l), a particular case of the compounds of formula (I): 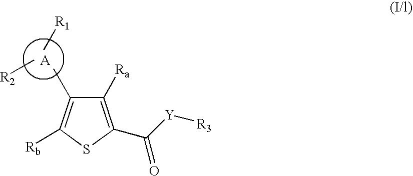
in which A, R₁, R₂, Rₐ, R_{b}, R₃ and Y are as defined above,
which compounds of formula (I/l) can be treated, for example with 2,4-bis(4-methoxyphenyl)-1,3-dithia-2,4-diphosphetane-2,4-disulphide, to yield to the compounds of formula (I/m), a particular case of the compounds of formula (I): 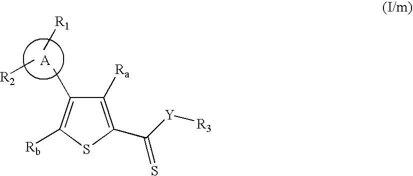
in which Rₐ, R_{b}, R₁, R₂, A, Y and R₃ are as defined in the formula (I),
the compounds (I/g) to (I/m) together forming compounds of the invention, which are purified, if appropriate, according to a conventional purification technique, which can, if desired, be separated into their various isomers according to a conventional separating technique, and which are converted, if appropriate, into their addition salts with a pharmaceutically acceptable acid or base.

19- Pharmaceutical compositions comprising, as active principle, at least one compound of formula (I) according to claim 1, alone or in combination with one or more pharmaceutically acceptable, nontoxic, inert excipients or vehicles.

20- Pharmaceutical compositions according to claim 19 useful for the prevention or the treatment of pathologies requiring the action of an inhibitor of metalloproteinase-12 and/or of metalloproteinase-13.

21- Pharmaceutical compositions according to claim 19, useful for the prevention or treatment of pathologies requiring the action of an inhibitor of metalloproteinase-12.

22- Pharmaceutical compositions according to claim 19, useful for the prevention or treatment of respiratory pathologies selected from chronic obstructive pulmonary disease (COPD), emphysema, chronic bronchitis, chronic pulmonary inflammation, asthma, mucoviscidosis, acute respiratory distress syndrome (ARDS), respiratory allergies, including allergic rhinitis, and diseases related to the production of TNFα, including severe fibrotic pulmonary diseases, pulmonary sarcoidosis and silicosis.

23- Pharmaceutical compositions according to claim 19, useful for the prevention or treatment of pathologies related to metalloproteinase-13 selected from cancer, osteoporosis, osteoarthritis, arthritis, rheumatoid arthritis, atherosclerosis, multiple sclerosis or cardiac insufficiency.

24- Pharmaceutical compositions according to claim 19, useful for the prevention or treatment of chronic obstructive pulmonary disease, of emphysema and of chronic bronchitis.

25- Pharmaceutical compositions according to claim 19, useful for the prevention or treatment of smoking-related emphysema.

26- Pharmaceutical compositions according to claim 19, useful for the prevention or treatment of asthma.
